SEQUENCE LISTING (71 sequences)

SEQ ID NO: 1 - protein, 8 aa, synthetic construct
GYTFTNYG

SEQ ID NO: 2 - protein, 8 aa, synthetic construct
INTYTGEP

SEQ ID NO: 3 - protein, 13 aa, synthetic construct
ARLGYGKFYVMDY

SEQ ID NO: 4 - protein, 11 aa, synthetic construct
QSLVHSNGNTY

SEQ ID NO: 5 - skipped

SEQ ID NO: 6 - protein, 9 aa, synthetic construct
SQSTHVTWT

SEQ ID NO: 7 - protein, 12 aa, unidentified
FRGQEDASPDRF

SEQ ID NO: 8 - protein, 120 aa, synthetic construct
QIQLVQSGPELKKPGETVKISCKASGYTFTNYGMNWVKQAPGKGLKWMGWINTYTGEPTYADDFKGRFAFSLETSASTAYLQINNLRNEDMSTYFCARLGYGKFYVMDYWGQGTSVTVSS

SEQ ID NO: 9 - protein, 120 aa, synthetic construct
QIQLVQSGSELKKPGASVKISCKASGYTFTNYGMNWVRQAPGQGLEWMGWINTYTGEPTYADDFKGRFVFSLDTSVSTAYLQISSLKAEDTSVYFCARLGYGKFYVMDYWGQGTSVTVSS

SEQ ID NO: 10 - protein, 120 aa, synthetic construct
QIQLVQSGSELKKPGASVKISCKASGYTFTNYGMNWVRQAPGQGLEWMGWINTYTGEPTYAQGFTGRFVFSLDTSVSTAYLQISSLKAEDTSVYFCARLGYGKFYVMDYWGQGTSVTVSS

SEQ ID NO: 11 - protein, 120 aa, synthetic construct
QIQLVQSGPELKKPGASVKISCKASGYTFTNYGMNWVRQAPGQGLKWMGWINTYTGEPTYADDFTGRFVFSLDTSVSTAYLQISSLKAEDTSVYFCARLGYGKFYVMDYWGQGTSVTVSS

SEQ ID NO: 12 - protein, 120 aa, synthetic construct
QIQLVQSGSELKKPGASVKISCKASGYTFTNYGMNWVRQAPGQGLKWMGWINTYTGEPTYADDFKGRFVFSLDTSVSTAYLQISSLKAEDTSVYFCARLGYGKFYVMDYWGQGTSVTVSS

SEQ ID NO: 13 - protein, 120 aa, synthetic construct
QIQLVQSGPELKKPGASVKISCKASGYTFTNYGMNWVKQAPGQGLKWMGWINTYTGEPTYAQGFTGRFVFSLDTSVSTAYLQISSLKAEDTSTYFCARLGYGKFYVMDYWGQGTSVTVSS

SEQ ID NO: 14 - protein, 120 aa, synthetic construct
QIQLVQSGPELKKPGASVKISCKASGYTFTSYAMNWVKQAPGQGLKWMGWINTYTGEPTYADDFKGRFVFSLDTSVSTAYLQISSLKAEDTSVYFCARLGYGKFYVMDYWGQGTSVTVSS

SEQ ID NO: 15 - protein, 120 aa, synthetic construct
QIQLVQSGHEVKQPGASVKISCKASGYTFTNYGMNWVPQAPGQGLEWMGWINTYTGEPTYADDFKGRFVFSLDTSASTAYLQISSLKAEDMSMYFCARLGYGKFYVMDYWGQGTSVTVSS

SEQ ID NO: 16 - protein, 120 aa, synthetic construct
QIQLVQSGHEVKQPGASVKISCKASGYTFTNYGMNWVPQAPGQGLEWMGWINTYTGEPTYAQGFTGRFVFSLDTSASTAYLQISSLKAEDMSMYFCARLGYGKFYVMDYWGQGTSVTVSS

SEQ ID NO: 17 - protein, 120 aa, synthetic construct
QIQLVQSGHEVKQPGASVKISCKASGYTFTNYGMNWVKQAPGQGLKWMGWINTYTGEPTYADDFTGRFVFSLDTSASTAYLQISSLKAEDMSMYFCARLGYGKFYVMDYWGQGTSVTVSS

SEQ ID NO: 18 - protein, 120 aa, synthetic construct
QIQLVQSGPEVKQPGASVKISCKASGYTFTNYGMNWVPQAPGQGLKWMGWINTYTGEPTYADDFTGRFVFSLDTSASTAYLQISSLKAEDMSMYFCARLGYGKFYVMDYWGQGTSVTVSS

SEQ ID NO: 19 - protein, 120 aa, synthetic construct
QIQLVQSGPEVKQPGASVKISCKASGYTFTNYGMNWVKQAPGQGLKWMGWINTYTGEPTYAQGFTGRFVFSLDTSASTAYLQISSLKAEDMSTYFCARLGYGKFYVMDYWGQGTSVTVSS

SEQ ID NO: 20 - protein, 120 aa, synthetic construct
QIQLVQSGPEVKQPGASVKISCKASGYSFTTYGMNWVKQAPGQGLEWMGWINTYTGEPTYADDFKGRFVFSLDTSASTAYLQISSLKAEDMSTYFCARLGYGKFYVMDYWGQGTSVTVSS

SEQ ID NO: 21 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVTWTFGGGTKLEIK

SEQ ID NO: 22 - protein, 112 aa, synthetic construct
DVVMTQSPLSLPVTLGQPASISCRSSQSLVHSNGNTYLNWYQQRPGQSPRLLIYKVSNRFSGVPDRFSGSGSGTDFTLKISRVEAEDVGVYFCSQSTHVTWTFGGGTKLEIK

SEQ ID NO: 23 - protein, 112 aa, synthetic construct
DVVMTQSPLSLPVTLGQPASISCRSSQSLVHSDGNTYLHWYQQRPGQSPRLLIYKVSNRFSGVPDRFSGSGSGTDFTLKISRVEAEDVGVYFCSQSTHVTWTFGGGTKLEIK

SEQ ID NO: 24 - protein, 112 aa, synthetic construct
DVVMTQSPLSLPVTLGQPASISCRSSQSLVHSNGNTYLHWYQQRPGQSPRLLIYKVSNRDSGVPDRFSGSGSGTDFTLKISRVEAEDVGVYFCSQSTHVTWTFGGGTKLEIK

SEQ ID NO: 25 - protein, 112 aa, synthetic construct
DVVMTQSPLSLPVTLGQPASISCRSSQSLVHSNGNTYLHWFQQRPGQSPRLLIYKVSNRFSGVPDRFSGSGSGTDFTLKISRVEAEDVGVYFCSQSTHVTWTFGGGTKLEIK

SEQ ID NO: 26 - protein, 112 aa, synthetic construct
DIVMTQTPLSLSVTPGQPASISCKSSQSLVHSNGNTYLHWYLQKPGQSPQLLIYKVSNRFSGVPDRFSGSGSGTDFTLKISRVEAEDVGVYYCSQSTHVTWTFGGGTKLEIK

SEQ ID NO: 27 - protein, 112 aa, synthetic construct
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVTWTFGGGTKLEIK

SEQ ID NO: 28 - protein, 112 aa, synthetic construct
DVVMTQSPLSLPVTLGQPASISCRSSQSLVHSNGNTYLHWFLQRPGQSPRLLIYKVSNRFSGVPDRFSGSGSGTDFTLKISRVEAEDVGVYYCSQSTHVTWTFGGGTKLEIK

SEQ ID NO: 29 - protein, 112 aa, synthetic construct
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVTWTFGGGTKLEIK

SEQ ID NO: 30 - protein, 5 aa, synthetic construct
GGGGS

SEQ ID NO: 31 - protein, 6 aa, synthetic construct
HHHHHH

SEQ ID NO: 32 - DNA, 20 bp, synthetic construct
  source 1..20: mol_type = other DNA
gtgaaggcgtctcaaacagg

SEQ ID NO: 33 - DNA, 19 bp, synthetic construct
  source 1..19: mol_type = other DNA
gaagcggtcaagggcatct

SEQ ID NO: 34 - DNA, 20 bp, synthetic construct
  source 1..20: mol_type = other DNA
ttggtgatgggaggatgact

SEQ ID NO: 35 - DNA, 20 bp, synthetic construct
  source 1..20: mol_type = other DNA
ggccagtgactcagcagaaa

SEQ ID NO: 36 - DNA, 22 bp, synthetic construct
  source 1..22: mol_type = other DNA
aacaccctggtctggaagtacg

SEQ ID NO: 37 - DNA, 22 bp, synthetic construct
  source 1..22: mol_type = other DNA
tcgttggacagccttcaagacc

SEQ ID NO: 38 - DNA, 21 bp, synthetic construct
  source 1..21: mol_type = other DNA
ctcagacacatgcctacacct

SEQ ID NO: 39 - DNA, 20 bp, synthetic construct
  source 1..20: mol_type = other DNA
gaacatctctgctcgtcgct

SEQ ID NO: 40 - DNA, 22 bp, synthetic construct
  source 1..22: mol_type = other DNA
aaaccccttggaaatagtcagc

SEQ ID NO: 41 - DNA, 22 bp, synthetic construct
  source 1..22: mol_type = other DNA
ggaaagccaaatcccagatgag

SEQ ID NO: 42 - DNA, 22 bp, synthetic construct
  source 1..22: mol_type = other DNA
gcctccgagagctatatgatcg

SEQ ID NO: 43 - DNA, 20 bp, synthetic construct
  source 1..20: mol_type = other DNA
tcactccgtagaagccacca

SEQ ID NO: 44 - DNA, 20 bp, synthetic construct
  source 1..20: mol_type = other DNA
accagcgcatggacagttac

SEQ ID NO: 45 - DNA, 20 bp, synthetic construct
  source 1..20: mol_type = other DNA
cgagctggtcatggagttgt

SEQ ID NO: 46 - DNA, 20 bp, synthetic construct
  source 1..20: mol_type = other DNA
aagtcggtgaagaacgggca

SEQ ID NO: 47 - DNA, 20 bp, synthetic construct
  source 1..20: mol_type = other DNA
tgtgtagacgggttgttccc

SEQ ID NO: 48 - DNA, 20 bp, synthetic construct
  source 1..20: mol_type = other DNA
acccacagctcagcatcttc

SEQ ID NO: 49 - DNA, 20 bp, synthetic construct
  source 1..20: mol_type = other DNA
ggttcatggcaaaacgaggg

SEQ ID NO: 50 - DNA, 22 bp, synthetic construct
  source 1..22: mol_type = other DNA
gtctcctctgacttcaacagcg

SEQ ID NO: 51 - DNA, 22 bp, synthetic construct
  source 1..22: mol_type = other DNA
accaccctgttgctgtagccaa

SEQ ID NO: 52 - DNA, 22 bp, synthetic construct
  source 1..22: mol_type = other DNA
ccacagtccatagaatcctcgg

SEQ ID NO: 53 - DNA, 22 bp, synthetic construct
  source 1..22: mol_type = other DNA
tgcctgtccttcaggtactgca

SEQ ID NO: 54 - DNA, 19 bp, synthetic construct
  source 1..19: mol_type = other DNA
agcgacggttctacgtcca

SEQ ID NO: 55 - DNA, 19 bp, synthetic construct
  source 1..19: mol_type = other DNA
gcgactgtcggaagtagcc

SEQ ID NO: 56 - DNA, 22 bp, synthetic construct
  source 1..22: mol_type = other DNA
cctcagcatcttatccgagtgg

SEQ ID NO: 57 - DNA, 22 bp, synthetic construct
  source 1..22: mol_type = other DNA
tggatggtggtacagtcagagc

SEQ ID NO: 58 - DNA, 21 bp, synthetic construct
  source 1..21: mol_type = other DNA
aacatgcccgacccaattaca

SEQ ID NO: 59 - DNA, 21 bp, synthetic construct
  source 1..21: mol_type = other DNA
gtgtttctgcatacctcatgg

SEQ ID NO: 60 - DNA, 22 bp, synthetic construct
  source 1..22: mol_type = other DNA
tgagttccctcagccgttacct

SEQ ID NO: 61 - DNA, 22 bp, synthetic construct
  source 1..22: mol_type = other DNA
gaggtttcctctggtcctggta

SEQ ID NO: 62 - DNA, 20 bp, synthetic construct
  source 1..20: mol_type = other DNA
aggggcaggttctggtattg

SEQ ID NO: 63 - DNA, 20 bp, synthetic construct
  source 1..20: mol_type = other DNA
tgttgatgccttcacagcgt

SEQ ID NO: 64 - DNA, 22 bp, synthetic construct
  source 1..22: mol_type = other DNA
gctttgccaacatcagcagcga

SEQ ID NO: 65 - DNA, 22 bp, synthetic construct
  source 1..22: mol_type = other DNA
aggagggtcttcaggttggtgt

SEQ ID NO: 66 - DNA, 22 bp, synthetic construct
  source 1..22: mol_type = other DNA
ggactgtgtctcctgccagaat

SEQ ID NO: 67 - DNA, 22 bp, synthetic construct
  source 1..22: mol_type = other DNA
ggcagacattctggatggcact

SEQ ID NO: 68 - DNA, 22 bp, synthetic construct
  source 1..22: mol_type = other DNA
actggtgtgacaccaagaggtc

SEQ ID NO: 69 - DNA, 22 bp, synthetic construct
  source 1..22: mol_type = other DNA
ccacaggtgatcctcaaacacg

SEQ ID NO: 70 - DNA, 22 bp, synthetic construct
  source 1..22: mol_type = other DNA
tgatacgcctgagtggctgtct

SEQ ID NO: 71 - DNA, 22 bp, synthetic construct
  source 1..22: mol_type = other DNA
cacaagagcagtgagcgctgaa

(19) United States
(12) Patent Application Publication
(10) Pub. No.: US 2026/0365724 A1
(43) Pub. Date: Nov. 19, 2026

(54) TREATMENT OF EPIDERMAL GROWTH FACTOR RECEPTOR MUTANT NON-SMALL CELL LUNG CANCER

(71) Applicant: Brown University, Providence, RI (US)

(72) Inventors: Jack A. Elias, Providence, RI (US); Suchitra Kamle, Providence, RI (US); Chun Geun Lee, Woodbridge, CT (US); Bing Ma, Branford, CT (US)

(21) Appl. No.: 19/130,540

(22) PCT Filed: Nov. 28, 2023
(86) PCT No.: PCT/US2023/081359

§ 371 Date: May 15, 2025

Related U.S. Application Data
(60) Provisional application No. 63/385,178, filed on Nov. 28, 2022.
(60) Provisional application No. 63/479,319, filed on Jan. 10, 2023.

(51) Int. Cl. C07K 16/28; A61K 31/506; A61K 31/517; A61K 31/7048; A61K 39/00; A61P 35/00; G01N 33/68

(52) U.S. Cl. CPC C07K 16/28; A61K 31/506; A61K 31/517; A61K 31/7048; A61P 35/00; G01N 33/6893; A61K 2039/505; C07K 2317/565; C07K 2317/76; G01N 2333/705; G01N 2800/56

(57) ABSTRACT

Although patients with EGFR mutant NSCLC frequently respond to initial therapy with tyrosine kinase inhibitors (TKIs), most then develop therapeutic resistance. It is demonstrated herein that: (i) CHI3L1 activates a pathway (the HIPPO/Yap/Taz pathway) that plays an important role in the pathogenesis of TKI resistance and can serve as a biomarker of EGFR mutant lung cancer progression; (ii) inhibitors of CHI3L1 block this therapeutic resistance and act as a therapeutic in EGFR mutant NSCLC; and (iii) CHI3L1 inhibitors can interact in a synergistic manner with TKI in the treatment of EGFR mutant NSCLC.

### FIELD OF THE INVENTION

The embodiments of the present invention relate to methods for the treatment of epidermal growth factor receptor (EGFR) mutant non-small cell lung cancer (NSCLC) that address the therapeutic resistance that develops in these patients when treated with tyrosine kinase inhibitors (TKIs).

### BACKGROUND OF THE INVENTION

Lung cancer is a leading cause of cancer-related deaths worldwide.¹ According to the American Cancer Society, the two main types of lung cancer are small cell lung cancer (SCLC) and non-small cell lung cancer (NSCLC). NSCLC accounts for more than 80% of all lung cancers, with the majority of patients presenting with advanced disease at the time of diagnosis.²

About 10⁻³⁰% of patients with NSCLC harbor mutations, mainly in exons 18-21, of the epidermal growth factor receptor (EGFR) gene, which is considered an oncogenic driver.³ Among these, the exon 19 deletion mutation (p. E746-A750del) and exon 21-point mutation (p.L858R) are the most frequent, accounting for more than 85% of EGFR mutations. Other EGFR mutations are considered uncommon.⁴ The function of EGFR is to help cells grow and divide. A person with EGFR-positive lung cancer has EGFR proteins that are constantly signaling for cells to grow and divide.

NSCLCs that harbor mutations within the EGFR gene are sensitive to the tyrosine kinase inhibitors (EGFR-TKIs) such as gefitinib and erlotinib. Accordingly, TKIs represent the first-line treatment and targeted therapy for patients with metastatic NSCLC harboring EGFR mutations.⁵ Unfortunately, the response to EGFR-TKIs is severely impaired in almost all patients by the unavoidable emergence of resistance to targeted therapy within 10⁻¹² months. Drug resistance occurs in these patients, and due to the failure of the immune surveillance, cancer cell growth continues.^{6,7} All patients treated with these drugs will acquire resistance, most commonly as a result of a secondary mutation within EGFR (T790M).⁸

Accordingly, there is a need for a novel approach to the treatment of EGFR mutant NSCLC and the therapeutic resistance that develops in these patients when treated with tyrosine kinase inhibitors (TKIs).

### BRIEF SUMMARY OF THE INVENTION

In the present disclosure, a novel mechanism is identified that underlies the contributions of epidermal growth factor receptor (EGFR) mutations to the pathogenesis of NSCLC. Although patients with EGFR mutant NSCLC frequently respond to initial therapy with tyrosine kinase inhibitors (TKIs), most then develop therapeutic resistance. In fact, the development of TKI resistance is a rate limiting step in the treatment of EGFR mutant NSCLC. The present disclosure defines the mechanisms that contribute to this resistance to TKI and provides methods for: (a) the treatment of EGFR mutant NSCLC, and (b) the treatment of TKI resistance in EGFR mutant NSCLC using at least one CHI3L1 inhibitor that can be at least one antibody or a small molecule CHI3L1 inhibitor. In these treatment methods, the anti-CHI3L1 antibody reagent is effective in its own right and interacts synergistically with one or more TKI. The present disclosure also provides a biomarker of EGFR mutant NSCLC progression.

The present disclosure provides a method of treating EGFR mutant NSCLC in a subject in need thereof, the method comprising administering an inhibitor of CHI3L1. In some embodiments, the inhibitor of CHI3L1 is an antibody, antibody reagent, antigen-binding fragment thereof, or CAR that specifically binds to the epitope of SEQ ID NO: 7. In some embodiment, the inhibitor of CHI3L1 is an antibody comprising the complementarity determining regions (CDRs) of: (a) a heavy chain CDR1 having the amino acid sequence of SEQ ID NO: 1; (b) a heavy chain CDR2 having the amino acid sequence of SEQ ID NO: 2; (c) a heavy chain CDR3 having the amino acid sequence of SEQ ID NO: 3; (d) a light chain CDR1 having the amino acid sequence of SEQ ID NO: 4; (e) a light chain CDR2 having the amino acid sequence of SEQ ID NO: 5; and (f) a light chain CDR3 having the amino acid sequence of SEQ ID NO: 6. In some embodiment, the inhibitor of CHI3L1 is an antibody comprising a light heavy chain sequence of any one of SEQ ID NO: 8-20. In some embodiment, the inhibitor of CHI3L1 is an antibody comprising a light chain sequence of any one of SEQ ID NO: 21-29. In some embodiment, the inhibitor of CHI3L1 is an antibody comprising a heavy chain sequence of any one of SEQ ID NO: 8-20 and a light chain sequence of any one of SEQ ID NO: 21-29. In some embodiment, the inhibitor of CHI3L1 is an inhibitor of CHI3L1 and chitinase 1. In some embodiment, the inhibitor of CHI3L1 and chitinase 1 is kasugamycin or a derivative, analog, or variant thereof. In some embodiment, the inhibitor of CHI3L1 and chitinase 1 is kasugamycin.

In an alternative aspect, the method of treating EGFR mutant NSCLC comprises administering one of the above-listed CHI3L1 inhibitor along with an EGFR-tyrosine kinase inhibitor (TKI). In some embodiment, the EGFR-TKI is selected from the group consisting of: afatinib, erlotinib, gefitinib, olmutinib, osimertinib, rociletinib, cetuximab, panitumumab, dacomitinib, lapatinib, necitumumab, mobocertinib, and vandetanib. In some embodiment, the EGFR-TKI is erlotinib, gefitinib or osimertinib.

In some embodiments of the method of treating EGFR mutant NSCLC of the present disclosure, the subject is afflicted with advanced EGFR mutant NSCLC and is no longer responding to TKI treatments. In some embodiments, the subject is determined to have an elevated level of CHI3L1. In some embodiments, the subject is determined to have a level of CHI3L1 that is increased compared to a prior assessment of the level in that subject. In some embodiments, the subject is determined to have a level of CHI3L1 that is increased compared to a reference level. In some embodiments, the CHI3L1 is circulating CHI3L1. In some embodiments, the subject afflicted with metastatic EGFR mutant NSCLC is determined to have a level of CHI3L1 that is increased compared to the subject's levels prior to metastasis. In some embodiments, the subject afflicted with metastatic EGFR mutant NSCLC is determined to have a level of CHI3L1 that is increased compared to the levels in patients with EGFR mutant lung cancer without metastasis.

In an alternative aspect, the present disclosure provides a composition for use in the treatment of EGFR mutant NSCLC in a subject in need thereof, the composition comprising one of the above-listed CHI3L1 inhibitor. In some embodiments, the composition further comprises an EGFR-tyrosine kinase inhibitor (TKI). In some embodiment, the EGFR-TKI is selected from the group consisting of: afatinib, erlotinib, gefitinib, olmutinib, osimertinib, rociletinib, cetuximab, panitumumab, dacomitinib, lapatinib, necitumumab, mobocertinib, and vandetanib. In some embodiment, the EGFR-TKI is erlotinib, gefitinib or osimertinib. In some embodiments, the subject is afflicted with advanced EGFR mutant NSCLC and is no longer responding to TKI treatments. In some embodiments, the subject is determined to have an elevated level of CHI3L1. In some embodiments, the subject is determined to have a level of CHI3L1 that is increased compared to a prior assessment of the level in that subject. In some embodiments, the subject is determined to have a level of CHI3L1 that is increased compared to a reference level. In some embodiments, the CHI3L1 is circulating CHI3L1. In some embodiments, the subject afflicted with metastatic EGFR mutant NSCLC is determined to have a level of CHI3L1 that is increased compared to the subject's levels prior to metastasis. In some embodiments, the subject afflicted with metastatic EGFR mutant NSCLC is determined to have a level of CHI3L1 that is increased compared to the levels in patients with EGFR mutant lung cancer without metastasis.

In an alternative aspect, the present disclosure provides a method of assessing the progression of EGFR mutant NSCLC in a subject afflicted with, or at risk of, EGFR mutant NSCLC using CHI3L1 as a biomarker, the method comprising assessing the level of CHI3L1 in the subject, wherein the level of CHI3L1 correlates directly with disease progression and inversely with disease-free interval and survival. In some embodiment, the subject is determined to have a level of CHI3L1 that is compared to a prior assessment of the level in that subject. In some embodiment, the subject is determined to have a level of CHI3L1 that is increased compared to a reference level. In some embodiment, the level of CHI3L1 is the circulating level of CHI3L1.

Other implementations are also described and recited herein.

### BRIEF DESCRIPTION OF THE DRAWINGS

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1 shows that TGF-α (top panels) and CHI3L1 (bottom panels) stimulate CHI3L1 mRNA and EGFR mRNA accumulation in cells with wild-type EGFR. CHI3L1 stimulation of CHI3L1 represents CHI3L1 autoinduction. Gene expression was evaluated by reverse transcriptase polymerase chain reaction (RT-PCR). Values are mean±SEM. **P<0.01, ***P<0.001, ****P<0.00001.

FIG. 2 shows that CHI3L1 stimulates the expression of EGFR in cells with wild-type EGFR. Gene expression was evaluated by RT-PCR. Values are mean±SEM. **P<0.01, ****P<0.00001.

FIG. 3 schematically illustrates common EGFR mutations (Exon 19, L858R). There are a number of EGFR mutations that play a role in the pathogenesis of EGFR mutant non-small cell lung cancer (NSCLC); some are common, and some are not. The common ones include L858R and mutations of exon 19.

FIG. 4 shows that CHI3L1 stimulates CHI3L1 autoinduction and components of EGFR axis in cells with “common” EGFR mutations (L858R). Gene expression was evaluated by RT-PCR. Values are mean±SEM. *P<0.05, **P<0.01, ****P<0.0001.

FIG. 5 shows the dose dependence of the effects of CHI3L1 (top panels) and TGF-α (bottom panels) on CHI3L1 autoinduction and EGFR in cells with EGFR mutations. Gene expression was evaluated by RT-PCR. Values are mean±SEM. *P<0.05, **P<0.01, ***P<0.001, ****P<0.00001.

FIG. 6 shows the CHI3L1 stimulation of EGFR in cells with other EGFR mutations. These studies also demonstrate that a monoclonal antibody against CHI3L1 abrogates these inductive effects. Gene expression was evaluated by RT-PCR. Values are mean±SEM. ***P<0.001.

FIG. 7 shows the exaggerated baseline accumulation of CHI3L1 and EGFR in epithelial cells with “common” EGFR mutations using double label immunocytochemistry (ICC). Exaggerated levels of CHI3L1 were seen in nuclei of these cells and exaggerated levels of EGFR were seen in their cytoplasm. DAPI was used to stain nuclei.

FIG. 8A-C shows that gefitinib inhibits the murine pulmonary accumulation of pleural colonies of B16-F10 malignant melanoma cells and BAL CHI3L1. The B16-F10 metastasis model was used to define the effects of the tyrosine kinase inhibitor (TKI) gefitinib on the pulmonary spread of malignant melanoma and the accumulation of bronchoalveolar lavage (BAL) CHI3L1 during these circumstances. These experiments compare mice that got gefitinib versus its DMSO vehicle control and demonstrate that gefitinib is a potent inhibitor of metastasis and the production of CHI3L1 in lungs with melanoma metastasis (FIG. 8A). FIG. 8B provides the quantitation of pleural colony numbers in indicated conditions. FIG. 8C provides the BAL levels of CHI3L1 assessed by ELISA assay. Values are mean±SEM. **P<0.01.

FIG. 9 shows that erlotinib inhibits the murine pulmonary accumulation of pleural colonies of B16-F10 cells. The B16-F10 metastasis model was used to define the effects of the tyrosine kinase inhibitor (TKI) erlotinib or its DMSO vehicle control on the pulmonary spread of malignant melanoma in WT mice (Tg−) and mice in which CHI3L1 is overexpressed in the lung in a transgenic manner (Tg+). The results demonstrate that the transgenic overexpression of CHI3L1 increases B16-F10 metastasis and that erlotinib inhibits B16-F10 cell metastasis even when CHI3L1 is overexpressed in the lung. Values are mean±SEM. *P<0.05,**P<0.01.

FIG. 10A-B shows that anti-CHI3L1 (FRG) and osimertinib inhibit the accumulation of pleural colonies of B16-F10 Cells. FIG. 10A shows lungs with melanoma metastasis. FIG. 10B provides the quantitation of pleural colony numbers. Values are mean±SEM. **P<0.01, ns=not significant.

FIG. 11 schematically illustrates the contributions of CHI3L1 to the EGFR axis. EGFR activation stimulates CHI3L1 via Stat 3. The CHI3L1 then feeds back to stimulate the expression, accumulation and phosphorylation(P) of EGFR and its ligands (EGF, AREG, TGF-α) as noted. CHI3L1 also feeds back to inhibit and ubiquitinate (U) the tumor suppressors P53 and PTEN, which in turn inhibit P13K and MAPK activation.

FIG. 12A-D shows that the anti-CHI3L1 antibody, FRG, inhibits CHI3L1 autoinduction and CHI3L1 stimulation of components of the EGFR axis in cells with “common” EGFR mutations. These data demonstrate that CHI3L1 is a potent stimulator of CHI3L1 autoinduction (FIG. 12A) and EGFR (FIG. 12B), and its physiologic ligands TGF-α (FIG. 12C) and EGF (FIG. 12D) and that FRG is a potent inhibitor of these inductive events. Values are mean±SEM. *P<0.05, **P<0.01, ***P<0.001.

FIG. 13A-C shows the effect of anti-CHI3L1 and TKI (osimertinib), alone and in combination, on the EGFR axis in cells with “common” EGFR mutations. These data demonstrate that osimertinib and FRG in combination are more potent than each individually. Values are mean±SEM. *P<0.05, **P<0.01, ***P<0.001, ns=not significant.

FIG. 14A-B shows the combinatorial effects of low doses of anti-CHI3L1 (FRG) and TKI (osimertinib) on CHI3L1 and EGFR axis in cells with “common” EGFR mutations. These data demonstrate that osimertinib and FRG in combination, at doses that individually have no effect, interact in a synergistic manner to inhibit the expression of CHI3L1 (FIG. 14A) and EGFR (FIG. 14B), and that these inhibitory effects are more potent than the effects of each moiety individually. Values are mean±SEM. *P<0.05, **P<0.01, ***P<0.001, ns=not significant.

FIG. 15A-B shows the effect of anti-CHI3L1 (FRG) and TKI (gefitinib), alone and in combination, on the survival of cells with “common” EGFR mutations using terminal deoxynucleotidyl transferase dUTP nick end labeling (TUNEL) analysis. The results of the TUNEL staining can be seen histologically in FIG. 15A characterizing results in adherent cells and cells that formed spheroids. The TUNEL staining is quantitated in FIG. 15B. These data demonstrate that gefitinib and FRG in combination interact in a synergistic way to stimulate cell death and DNA injury and that these cell death effects are more potent than the effects induced by each moiety individually. Values are mean±SEM. ***P<0.001, ****P<0.0001.

FIG. 16A-B shows the effect of anti-CHI3L1 (FRG) and TKI (osimertinib), alone and in combination, on the survival of cells with “common” EGFR mutation using TUNEL analysis. The results of the TUNEL staining can be seen in the cytochemistry panels characterizing results in adherent cells in FIG. 16A. The nuclei of these cells are highlighted by blue DAPI staining. The TUNEL staining is quantitated in FIG. 16B. These data demonstrate that osimertinib and FRG in combination interact in a synergistic manner to stimulate cell death and DNA injury and that these cell death responses are more potent than the responses induced by each moiety individually. Values are mean±SEM. *** P<0.001, ****P<0.0001.

FIG. 17A-B shows the effect of anti-CHI3L1 (FRG) and gefitinib, alone and in combination, on pleural colonies in B16-F10 challenged mice. B16-F10 melanoma cells were injected into tail veins of wild type C57BL/6 mice and their lungs were removed and evaluated visually (FIG. 17A) and quantitatively (FIG. 17B) 14 days later. These data demonstrate that gefitinib and FRG in combination interact in a synergistic way to inhibit melanoma metastasis and that these inhibitory effects are more potent than the effects of each moiety individually. Values are mean±SEM. *P<0.05, **P<0.01, ***P<0.001, ****P<0.0001, ns=not significant.

FIG. 18A-D shows the effect of CHI3L1 stimulation of Yes-Associated Protein 1 (YAP1) in cells with WT EGFR expression and “common” EGFR mutations. Epithelial cells with wild type EGFR (A549 cells, shown in FIG. 18A and Calu3 cells, shown in FIG. 18B) and cells with mutant EGFR (H1975, shown in FIG. 18C and HCC827, shown in FIG. 18D) were incubated with rCHI3L1 or its vehicle control (NT) for 72 hours. qRT-PCR was employed to assess YAP-1 gene expression. CHI3L1 stimulated YAP-1 in all four experimental settings. Values are mean±SEM. **P<0.01, ***P<0.001.

FIG. 19A-D shows the effect of CHI3L1 on the expression of TAZ on the EGFR axis in cells with WT EGFR expression and “common” EGFR mutations. Epithelial cells with wild type EGFR (A549 cells, shown in FIG. 19A and Calu3 cells, shown in FIG. 19B) and cells with mutant EGFR (H1975, shown in FIG. 19C and HCC827, shown in FIG. 19D) were incubated with rCHI3L1 or its vehicle control (NT) for 72 hours. qRT-PCR was employed to assess TAZ gene expression. CHI3L1 stimulated TAZ in all four experimental settings. Values are mean±SEM. *P<0.05, **P<0.01, ***P<0.001.

FIG. 20A-D shows the effect of CHI3L1 stimulation on TEAD1 in the EGFR axis in cells with WT EGFR expression and “common” EGFR mutations. Epithelial cells with wild type EGFR (A549 cells, shown in FIG. 20A and Calu3 cells, shown in FIG. 20B) and cells with mutant EGFR (H1975, shown in FIG. 20C and HCC827, shown in FIG. 20D) were incubated with rCHI3L1 or its vehicle control (NT) for 72 hours. qRT-PCR was employed to assess TEAD1 gene expression. CHI3L1 stimulated TEAD1 in all four experimental settings. Values are mean±SEM. *P<0.05, **P<0.01.

FIG. 21 shows the immunohistochemical demonstration of the effects of CHI3L1 stimulation on YAP-1 in nuclei from NSCLC cells with EGFR “common” mutations. At baseline, YAP1 accumulation was not readily appreciated. When stimulated with rCHI3L1, nuclear YAP-1 was significantly induced. Treatment with FRG diminished the accumulation of YAP1 in the nuclei and the YAP1 that was seen was largely in a cytoplasmic location (orange arrow).

FIG. 22 shows the immunohistochemical demonstration of CHI3L1 stimulation of YAP-1 in nuclei from NSCLC cells with EGFR “common” mutations. At baseline, TAZ accumulation was not readily appreciated. When stimulated with rCHI3L1, nuclear TAZ was significantly induced. Treatment with FRG diminished the accumulation of TAZ in the nuclei and the TAZ in these cells.

FIG. 23 shows the immunohistochemical demonstration of the effects of CHI3L1 on the YAP-1 in nuclei from NSCLC cells with EGFR “common” mutations. At baseline, YAP1 accumulation was not readily appreciated. When stimulated with rCHI3L1, nuclear YAP-1 was significantly induced. Treatment with FRG diminished this YAP-1 accumulation.

FIG. 24A-B shows the CHI3L1 stimulation of Sox-mediated stemness in cells with wild type (Calu-3 cells) and mutant EGFR (H1975 cells). These data demonstrate that CHI3L1 stimulated Sox-2 (FIG. 24A) and Sox-9 (FIG. 24B) expression. Values represent the mean±SEM of a minimum of 3 determinations.

FIG. 25 schematically illustrates the contributions of CHI3L1 to EGFR signaling in tumor resistance and progression. EGFR activation stimulates CHI3L1 via Stat 3. CHI3L1 then feeds back to stimulate the expression and accumulation of EGFR and its ligands (EGF, AREG, TGF-α). CHI3L1 also feeds back to inhibit the tumor suppressors P53 and PTEN which in turn inhibit P13K and MAPK activation. This positive feedback loop also leads to activation of the Hippo/YAP-1/TAZ pathway, which contributes to cell survival, cell cycle progression and TKI resistance via down-stream genes such as Sox 2 and 9 which regulate stemness.

FIG. 26A-C shows the effect of the small molecule CHI3L1 inhibitor, kasugamycin (KSM), on CHI3L1 induction of CHI3L1 (CHI3L1 autoinduction; FIG. 26A) and CHI3L1 stimulation of the expression of EGFR (FIG. 26B) and YAP-1 (FIG. 26C). The figures compare the effects of recombinant CHI3L1 (250 ng/mL) and vehicle control on HCC827 cells (which have EGFR mutations) after 72 hours of treatment with and without the noted concentration of KSM (500 ng/mL). Values are mean±SEM. *P<0.05; **P<0.01, ***P<0.001, ****P<0.0001.

FIG. 27A-E illustrate the effects of the small molecule CHI3L1 inhibitor, kasugamycin (KSM), on CHI3L1 induction of CHI3L1 (CHI3L1 autoinduction; FIG. 27A) and CHI3L1 stimulation of the expression of EGFR (FIG. 27B), and its physiologic ligands TGF-α (FIG. 27C), EGF (FIG. 27D), and YAP-1 (FIG. 27E). The figures compare the effects of recombinant CHI3L1 (250 ng/mL) and vehicle control on H1975 cells (which have EGFR mutations) after 72 hours of treatment with and without the noted concentration of KSM (500 ng/mL). Values are mean±SEM. *P<0.05, **P<0.01, ***P<0.001.

FIG. 28A-F illustrates that EGFR activation induced the expression and production of CHI3L1. NHBE cells were incubated in the presence or absence of EGF or TGF-α. Triplicate determinations of the levels of mRNA encoding CHI3L1 at the noted time points can be seen in FIG. 28A. The accumulation of CHI3L1 protein at 72 hours can be seen with the Western blotting in FIG. 28B and IF staining can be seen in FIG. 28C. In FIG. 28D-F, similar evaluations were undertaken with A549 cells. The values in FIG. 28A and FIG. 28D are the mean±SEM of triplicate evaluations and the data in FIG. 28B, FIG. 28C, FIG. 28E, and FIG. 28F are representative of three similar evaluations. In FIG. 28C and FIG. 28F, blue is DAPI, green is cytoskeleton, and red is CHI3L1. ***P<0.001; ****P<0.0001 by t-test. Scale bar=25 μm applies to FIG. 28C and FIG. 28F.

FIG. 29 provides the dose response of TGF-α (10, 50, and 100 ng/mL) stimulation of CHI3L1 protein production by NHBE cells evaluated by Western analysis.

FIG. 30A-F illustrates the induction of the expression and production of CHI3L1 in cells with EGFR mutations. HCC827 cells were incubated in the presence or absence of EGF or TGF-α. Triplicate determinations of the levels of mRNA encoding CHI3L1 at the noted time points can be seen in FIG. 30A. The accumulation of CHI3L1 protein at 72 hours can be seen with the Western blotting in FIG. 30A and double label IF is seen in FIG. 30C. In FIG. 30D-F, similar evaluations were undertaken with H1975 cells. The values in FIG. 30A and FIG. 30D are the mean±SEM of triplicate evaluations and the data in FIG. 30B, FIG. 30C, FIG. 30E, and FIG. 30F are representative of three similar evaluations. In FIG. 30C and FIG. 30F, blue is DAPI, green is cytoskeleton, and red is CHI3L1. **P<0.01; ***P<0.001 by t-test. Scale bar=25 μm applies to FIG. 30C and FIG. 30F.

FIG. 31 provides the dose response of EGF (5, 10, and 20 ng/mL) stimulation of CHI3L1 protein production by HCC827 cells evaluated by Western analysis.

FIG. 32 shows the immunohistochemical evaluation of baseline CHI3L1 (red) in nuclei and cytoplasm in HCC827 cells (DAPI=blue, cytoskeleton=green, CHI3L1=red). Magnification=20×.

FIG. 33A-B provides the ELISA-based comparison of the levels of CHI3L1 secreted by (A) EGF and (B) TGF-α stimulated NHBE, A549, H1975 and HCC827 cells incubated for 72 hours. ****P<0.001 by t-test. NT=no treatment.

FIG. 34A-L illustrates that CHI3L1 elicited a positive feedback loop in the EGFR Axis. H1975 cells were incubated with the noted concentration of rCHI3L1 and the levels of mRNA encoding EGFR, EGF and TGF-α were evaluated in FIG. 34A-C. The importance of CHI3L1 in these inductive events was assessed in FIG. 34D-F by comparing the expression of EGF, TGF-α and EGFR in cells incubated in the presence and absence of anti-CHI3L1 (FRG) antibody. HCC827 cells were similarly incubated with the noted concentrations of rCHI3L1 in the presence and absence of FRG in FIG. 34G-I. In FIG. 34J-L the levels of mRNA encoding EGFR, EGF and TGF-α were assessed in lungs from wild type (wt) mice, CHI3L1 null mutant mice (ko) and CHI3L1 overexpression transgenic mice (tg). The values in FIG. 34A-I represent the mean±SEM of a minimum of three evaluations under each condition. The values in FIG. 34J-L represent the mean±SEM of evaluations in lungs from a minimum of five (or more) animals. *P<0.05; **P<0.01; ***P<0.001 by One-way ANOVA with multiple comparisons.

FIG. 35A-C illustrates that CHI3L1 is auto induced in cells with WT and mutant EGFR. NHBE, A549, H1975, and HCC827 cells were incubated in the presence and absence of rCHI3L1 and the levels of mRNA encoding CHI3L1 and CHI3L1 protein accumulation were evaluated. The levels of mRNA encoding CHI3L1 were evaluated using qRT-PCR as illustrated in FIG. 35A. The levels of CHI3L1 protein accumulation can be seen in the Western blot evaluations and immunocytochemistry in FIG. 35B and FIG. 35C. The values in FIG. 35A represent the mean±SEM of at least triplicate evaluations. The values in FIG. 35B and FIG. 35C are representative of at least three similar evaluations. **P<0.05; ***P<0.01 by t-test. Scale bar=25 μm applies to FIG. 35C.

FIG. 36A-F shows the effects of CHI3L1 inhibitors and TKI, alone and in combination, on the EGFR axis in vitro. HCC827 cells were incubated with rCHI3L1 and the effects of osimertinib (OSM) and FRG on the expression of CHI3L1, EGF and EGFR were evaluated by qRT-PCR (FIG. 36A-C). In FIG. 36D and FIG. 36E, H1975 cells were incubated with low doses of osimertinib (OSM) and FRG, alone or in combination, and the levels of mRNA encoding CHI3L1 and EGF were evaluated. In FIG. 36F, H1975 cells were incubated with osimertinib (OSM) and FRG antibody, alone or in combination, and the levels of apoptosis were evaluated by TUNEL staining. *P<0.05; **P<0.01; ***P<0.001; ****P<0.0001 by ANOVA with multiple comparisons. ns=not significant.

FIG. 37 provides the TUNEL evaluation of H1975 cells incubated with FRG antibody and Gefitinib, alone and in combination. EGF (20 ng/mL) and Gefitinib (100 ng/mL). ***P<0.001; ****P<0.0001 by t-test.

FIG. 38A-F shows that FRG, an anti-CHI3L1 antibody, reversed therapeutic resistance to osimertinib and regulates tumor suppressor genes. HCC827 cells were chronically incubated with osimertinib to develop osimertinib resistant HCC8270R2 cells. The effects of CHI3L1 inhibitors and TKI, alone or in combination, were then evaluated by incubating the HCC8270R2 cells with osimertinib, alone or in combination with FRG and evaluating the levels of mRNA encoding CHI3L1, EGF and EGFR (FIG. 38A). The ability of osimertinib to induce HCC8270R2 cell death was also evaluated. In this experiment, HCC8270R2 cells were incubated with osimertinib and FRG, alone or in combination, and TUNEL evaluations were undertaken (FIG. 38B). Studies were also undertaken to determine if osimertinib and FRG, alone or in combination, altered the expression and or accumulation of tumor suppressor genes. The effects on KEAP1 mRNA and protein can be seen in FIG. 38C and FIG. 38D, respectively. The effects on RB1, P53 and PTEN are illustrated in FIG. 38E. *P<0.05; **P<0.01; ***P<0.001; ****P<0.0001 by ANOVA with multiple comparisons. ns=not significant. Scale bar=25 μm applies to FIG. 38B.

FIG. 39A-D shows that FRG, an anti-CHI3L1 antibody, and TKI interacted to inhibit tumor metastasis. WT mice B16-F10 were challenged with (B16) melanoma cells or control vehicle and treated with control IgG, FRG and or TKIs, alone or in combination. Melanoma lung metastasis was evaluated two weeks later. FIG. 39A shows representative lungs from mice treated with control IgG, FRG and/or gefitinib, alone or in combination.

FIG. 39B provides the number of pleural melanoma colonies quantitated in the lungs from the mice in FIG. 39A. FIG. 39C shows representative lungs from mice treated with IgG, FRG and osimertinib, alone and in combination. FIG. 39D provides the number of pleural melanoma colonies quantitated in the lungs from the mice in FIG. 39C. In FIG. 39A and FIG. 39C, each dot is representative of an individual animal. FIG. 39A and FIG. 39C are representative of at least three evaluations. The values in FIG. 39B and FIG. 39D represent the mean±SEM of the evaluations represented by the individual dots in the lungs from the experiments illustrated in FIG. 39A and FIG. 39C, respectively. *P<0.05; **P<0.01; ***P<0.001; ****P<0.0001 by ANOVA with multiple comparisons. ns=not significant.

### DETAILED DESCRIPTION OF THE INVENTION

It is to be appreciated that certain aspects, modes, embodiments, variations and features of the invention are described below in various levels of detail in order to provide a substantial understanding of the present invention.

### DEFINITIONS

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The abbreviation, “e.g.,” is derived from the Latin exempli gratia, and is used herein to indicate a non-limiting example. Thus, the abbreviation “e.g.,” is synonymous with the term “for example.”

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the phrase “therapeutically effective amount”, “effective amount” or “effective dose” refers to an amount that provides a therapeutic or aesthetic benefit in the treatment, prevention, or management of a tumor or malignancy, e.g., an amount that provides a statistically significant decrease in at least one symptom, sign, or marker of a tumor or malignancy. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, the term “administering,” refers to the placement of a CHI3L1 inhibitor such as an FRG antibody or Kasugamycin (KSM), as disclosed herein, into a subject by a method or route which results in at least partial delivery of the agent at a desired site. Pharmaceutical compositions comprising the compounds disclosed herein can be administered by any appropriate route which results in an effective treatment in the subject.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

A subject that has a cancer or a tumor is a subject having objectively measurable cancer cells or biomarker present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

A subject can be one who has been previously diagnosed with or identified as suffering from or having a condition in need of treatment (e.g., a cancer such as EGFR mutant NSCLC) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, or at risk of developing that condition.

The terms “decrease,” “reduced,” “reduction,” or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10⁻¹⁰⁰% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

As used herein, “engineered” refers to the aspect of having been manipulated by the hand of man. For example, an antibody, antibody reagent, antigen-binding portion thereof, or CAR is considered to be “engineered” when the sequence of the antibody, antibody reagent, antigen-binding portion thereof, or CAR is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems. The system described by Kabat et al. (Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Other boundaries defining CDRs overlapping with the Kabat CDRs have been described by Padlan (1995),⁹ MacCallum (1996),¹⁰ Chothia & Lesk (1987),¹¹ and Chothia (1989).¹² Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat defined CDRs.

The term “antigen-binding portion” of an antibody refers to one or more portions of an antibody as described herein, said portions) still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a dAb portion consisting of a VH domain or of VH, CH1, CH2, DH3, or VH, CH2, CH3 (dAbs, or single domain antibodies, comprising only V_{L} domains have also been shown to specifically bind to target epitopes). Although the two domains of the Fv portion, namely VL and VH, are encoded by separate genes, they may further be linked to one another using a synthetic linker, e.g., a poly-G4S amino acid sequence (“G4S” disclosed herein as SEQ ID NO: 24) (‘G4S’ disclosed as SEQ ID NO: 29 in U.S. Pat. No. 10,253,111), and recombinant methods, making it possible to prepare them as a single protein chain in which the VL and VH regions combine in order to form monovalent molecules (known as single chain Fv (ScFv)). The term “antigen-binding portion” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody, antigen-binding portion thereof, or CAR as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cysteine residue, a marker peptide and a C-terminal polyhistidinyl, e.g., hexahistidinyl tag (“hexahistidinyl tag” disclosed herein as SEQ ID NO: 25) (‘hexahistidinyl tag’ disclosed as SEQ ID NO: 30 in U.S. Pat. No. 10,253,111) in order to produce bivalent and biotinylated scFv molecules.

In some embodiments, the antibody, antibody reagent, antigen-binding portion thereof, or CAR described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least three, and more usually, at least five, about nine, or about 8-10 amino acids in a unique spatial conformation. An “epitope” includes the unit of structure conventionally bound by an immunoglobulin VHVL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide. In some embodiments, the CAR comprises an extracellular domain that binds CHI3L1, wherein the extracellular domain comprises a humanized or chimeric antibody or antigen-binding portion thereof.

The term “human antibody” refers to antibodies whose variable and constant regions correspond to or are derived from immunoglobulin sequences of the human germ line, as described, for example, by Kabat et al. (1991).¹³ However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, et al. (1991).¹⁴ According to particular embodiments, however, such recombinant human antibodies are subjected to in vitro mutagenesis (or to a somatic in vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody.¹⁵ These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

In some embodiments, the antibody, antibody reagent, antigen-binding portion thereof, and/or CAR as described herein can be a variant of a sequence described herein, e.g., a conservative substitution variant of an antibody polypeptide. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or portion thereof that retains activity, e.g., antigen-specific binding activity for the relevant target polypeptide, e.g., CHI3L1. A wide variety of PCR-based site-specific mutagenesis approaches are also known in the art and can be applied by the ordinarily skilled artisan.

Usually, the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin). The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody, antigen-binding portion thereof, or CAR as described herein. Such functional activities include binding to cancer cells and/or anti-cancer activity. Additionally, a polypeptide having functional activity means the polypeptide exhibits activity similar, but not necessarily identical to, an activity of a reference antibody, antigen-binding portion thereof, or CAR as described herein, including mature forms, as measured in a particular assay, such as, for example, a biological assay, with or without dose dependency. In the case where dose dependency does exist, it need not be identical to that of the reference antibody, antigen-binding portion thereof, or CAR, but rather substantially similar to the dose-dependence in a given activity as compared to the reference antibody, antigen-binding portion thereof, or CAR as described herein (i.e., the candidate polypeptide will exhibit greater activity, or not more than about 25-fold less, about 10-fold less, or about 3-fold less activity relative to the antibodies, antigen-binding portions, and/or CARs described herein).

In some embodiments, the antibody reagents (e.g., antibodies or CARs) described herein are not naturally occurring biomolecules. For example, a murine antibody raised against an antigen of human origin would not occur in nature absent human intervention and manipulation, e.g., manufacturing steps carried out by a human. Chimeric antibodies are also not naturally occurring biomolecules, e.g., in that they comprise sequences obtained from multiple species and assembled into a recombinant molecule. In certain particular embodiments, the human antibody reagents described herein are not naturally occurring biomolecules, e.g., fully human antibodies directed against a human antigen would be subject to negative selection in nature and are not naturally found in the human body.

In some embodiments, the antibody, antibody reagent, antigen-binding portion thereof, and/or CAR is an isolated polypeptide. In some embodiments, the antibody, antibody reagent, antigen-binding portion thereof, and/or CAR is a purified polypeptide. In some embodiments, the antibody, antibody reagent, antigen-binding portion thereof, and/or CAR is an engineered polypeptide.

“Avidity” is the measure of the strength of binding between an antigen-binding molecule (such as an antibody or antigen-binding portion thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D} of 10⁻⁵ to 10⁻¹² moles/liter or less, such as 10⁻⁷ to 10⁻¹² moles/liter or less, or 10⁻⁸ to 10⁻² moles/liter (i.e., with an association constant (K_{A}) of 10⁵ to 10¹² liter/moles or more, such as 10⁷ to 10¹² liter/moles or 10⁸ to 10¹² liter/moles). Any K_{D} value greater than 10⁻⁴ mol/liter (or any K_{A} value lower than 104 M-¹) is generally considered to indicate non-specific binding. The K_{D} for biological interactions which are considered meaningful (e.g., specific) are typically in the range of 10⁻¹⁰M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 μM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

Accordingly, as used herein, “selectively binds” or “specifically binds” refers to the ability of a peptide (e.g., an antibody, CAR, or portion thereof) described herein to bind to a target, such as an antigen present on the cell-surface of a cancer cell, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹²M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody, antigen-binding portion thereof, or CAR is said to specifically bind an antigen when it preferentially recognizes its target antigen in a complex mixture of proteins and/or macromolecules.

In some embodiments, an antibody, antigen-binding portion thereof, or CAR, as described herein, binds to CHI3L1 with a dissociation constant (K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10¹¹ M, 10⁻¹² M, or less. In some embodiments, an antibody, antigen-binding portion thereof, or CAR, as described herein, binds to CHI3L1 with a dissociation constant (K_{D}) of from about 10⁻⁵ M to 10⁻⁶ M. In some embodiments, an antibody, antigen-binding portion thereof, or CAR, as described herein, binds to CHI3L1 with a dissociation constant (K_{D}) of from about 10⁻⁶ M to 10⁻⁷ M. In some embodiments, an antibody, antigen-binding portion thereof, or CAR, as described herein, binds to CHI3L1 with a dissociation constant (K_{D}) of from about 10⁻⁷ M to 10⁻⁸ M. In some embodiments, an antibody, antigen-binding portion thereof, or CAR, as described herein, binds to CHI3L1 with a dissociation constant (K_{D}) of from about 10⁻⁸ M to 10⁻⁹ M. In some embodiments, an antibody, antigen-binding portion thereof, or CAR, as described herein, binds to CHI3L1 with a dissociation constant (K_{D}) of from about 10⁻⁹ M to 10⁻¹⁰ M. In some embodiments, an antibody, antigen-binding portion thereof, or CAR, as described herein, binds to CHI3L1 with a dissociation constant (K_{D}) of from about 10⁻¹⁰ M to 10⁻¹¹ M. In some embodiments, an antibody, antigen-binding portion thereof, or CAR, as described herein, binds to CHI3L1 with a dissociation constant (K_{D}) of from about 10⁻¹¹ M to 10⁻¹² M. In some embodiments, an antibody, antigen-binding portion thereof, or CAR, as described herein, binds to CHI3L1 with a dissociation constant (K_{D}) of less than 10⁻¹² M.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;¹⁶ The Encyclopedia of Molecular Cell Biology and Molecular Medicine;¹⁷ Molecular Biology and Biotechnology: a Comprehensive Desk Reference;¹⁸ Immunology;¹⁹ Janeway's Immunobiology;²⁰ Lewin's Genes XI;²¹ Molecular Cloning: A Laboratory Manual.;²² Basic Methods in Molecular Biology;²³ Laboratory Methods in Enzymology;²⁴ Current Protocols in Molecular Biology (CPMB);²⁵ Current Protocols in Protein Science (CPPS);²⁶ and Current Protocols in Immunology (CPI).²⁷ One of skill in the art can readily identify a chemotherapeutic agent of use.²⁸

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### CHI3L1 Inhibitors

As used herein, “inhibitor” refers to an agent which can decrease the expression and/or activity of a target, e.g., by at least 10% or more, e.g., by 10% or more, 50% or more, 70% or more, 80% or more, 90% or more, 95% or more, or 98% or more. The efficacy of an inhibitor of one or more targets, e.g., its ability to decrease the level and/or activity of the target can be determined, e.g., by measuring the level of an expression product of the target and/or the activity of the target. Methods for measuring the level of a given mRNA and/or polypeptide are known to one of skill in the art, e.g., RT-PCR with primers can be used to determine the level of RNA and Western blotting with an antibody can be used to determine the level of a polypeptide. The activity of, e.g., CHI3L1, can be determined using methods known in the art. In some embodiments of any of the aspects, the inhibitor can be an inhibitory nucleic acid; an aptamer; an antibody reagent; an antibody; or a small molecule. An inhibitor of a target described herein can inhibit the activity, expression, or accumulation of the target polypeptide.

Inhibitors of CHI3L1 (aka anti-CHI3L1) can include inhibitors that act directly on the target itself (e.g., that bind to the CHI3L1 protein or transcript, e.g., direct inhibitors) or inhibitors that act indirectly on the target, e.g., directly on one or more receptors for CHI3L1. As used herein, “CHI3L11,” “chintinase-3-like-1 protein,” or “YKL-40” refers to an approximately 40 kDa glycoprotein secreted by at least macrophages, chondrocytes, neutrophils, synovial cells, and some cancer cells. CHI3L1 does not have chitinase activity, is a Th2 promoting cytokine, has been linked to the AKT anti-apoptotic signaling pathway and induces the migration of astrocytes. The sequences of CHI3L1 expression products are known for a number of species, e.g., human CHI3L1 (NCBI Gene ID No: 1116) mRNA (SEQ ID NO: 59; NCBI Ref Seq: NM_001276.1 and SEQ ID NO: 26; NCBI Ref Seq: NM_001276.2) and polypeptide (SEQ ID NO: 27; NCBI Ref Seq: NP_001267.1 and SEQ ID NO: 28; NCBI Ref Seq: NP_001267.2) described and provided in U.S. Pat. No. 10,766,968, which is incorporated by reference herein in its entirety.

In some embodiments of any of the aspects the inhibitor of CHI3L1 is an anti-CHI3L1 antibody reagent. Anti-CHI3L1 antibody reagents useful in the methods of the instant disclosure include, but are not limited to, the anti-CHI3L1 antibody reagents disclosed in U.S. Pat. No. 10,253,111, which is incorporated by reference herein in its entirety.

### Anti-CHI3L1 Antibody Reagents

In some embodiments of any of the aspects, the anti-CHI3L1 antibody, antibody reagent, antigen-binding portion thereof, or CAR comprises heavy chain CDRs having the amino acid sequences of SEQ ID NOs: 1-3, or a conservative substitution variant of such amino acid sequence. In some embodiments of any of the aspects, the antibody, antibody reagent, antigen-binding portion thereof, or CAR comprises light chain CDRs having the amino acid sequences of SEQ ID NOs: 4-6, or a conservative substitution variant of such amino acid sequence. In some embodiments of any of the aspects, the antibody, antibody reagent, antigen-binding portion thereof, or CAR comprises heavy chain CDRs having the amino acid sequences of SEQ ID NOs: 1-3 and light chain CDRs having the amino acid sequences of SEQ ID NOs: 4-6, or a conservative substitution variant of such amino acid sequence. SEQ ID NOs: 1-6 are provided in Table 1.

TABLE 1

| Heavy Chain CDRs | Light Chain CDRs |
|---|---|
| GYTFTNYG (SEQ ID NO: 1) | QSLVHSNGNTY (SEQ ID NO: |
| | 4) |
| INTYTGEP (SEQ ID NO: 2) | KVS (SEQ ID NO: 5) |
| ARLGYGKFYVMDY (SEQ ID | SQSTHVTWT (SEQ ID NO: 6) |
| NO: 3) | |

In some embodiments of any of the aspects, the anti-CHI3L1 antibody, antibody reagent or antigen-binding fragment thereof binds specifically to the epitope of a CHI3L1 polypeptide, provided in Table 2.

TABLE 2
FRGQEDASPDRE (SEQ ID NO: 7)

In some embodiments of any of the aspects, the anti-CHI3L1 antibody, antibody reagent, antigen-binding portion thereof, or CAR comprises a heavy chain sequence having the amino acid sequences of any one of SEQ ID NOs: 8-20, or a conservative substitution variant of such amino acid sequence. In some embodiments of any of the aspects, the anti-CHI3L1 antibody, antibody reagent, antigen-binding portion thereof, or CAR comprises a light chain sequence having the amino acid sequences of any one of SEQ ID NOs: 21-29, or a conservative substitution variant of such amino acid sequence. In some embodiments of any of the aspects, the anti-CHI3L1 antibody, antibody reagent, antigen-binding portion thereof, or CAR comprises a heavy chain sequence having the amino acid sequences of any one of SEQ ID NOs: 8-20, or a conservative substitution variant of such amino acid sequence and a light chain sequence having the amino acid sequences of any one of SEQ ID NOs: 21-29, or a conservative substitution variant of such amino acid sequence. SEQ ID NOs: 8-29 are provided in Table 3.

TABLE 3
Heavy Chain Sequences
QIQLVQSGPELKKPGETVKISCKASGYTFTNYGMNWVKQAPGKGLKWMGWINTYTGEPTYADDFKGRFAFS
LETSASTAYLQINNLRNEDMSTYFCARLGYGKFYVMDYWGQGTSVTVSS (SEQ ID NO: 8)
QIQLVQSGSELKKPGASVKISCKASGYTFTNYGMNWVRQAPGQGLEWMGWINTYTGEPTYADDFKGRFVFS
LDTSVSTAYLQISSLKAEDTSVYFCARLGYGKFYVMDYWGQGTSVTVSS (SEQ ID NO: 9)
QIQLVQSGSELKKPGASVKISCKASGYTFTNYGMNWVRQAPGQGLEWMGWINTYTGEPTYAQGFTGRFVFS
LDTSVSTAYLQISSLKAEDTSVYFCARLGYGKFYVMDYWGQGTSVTVSS (SEQ ID NO: 10)
QIQLVQSGPELKKPGASVKISCKASGYTFTNYGMNWVRQAPGQGLKWMGWINTYTGEPTYADDFTGRFVFS
LDTSVSTAYLQISSLKAEDTSVYFCARLGYGKFYVMDYWGQGTSVTVSS (SEQ ID NO: 11)
QIQLVQSGSELKKPGASVKISCKASGYTFTNYGMNWVRQAPGQGLKWMGWINTYTGEPTYADDFKGRFVFS
LDTSVSTAYLQISSLKAEDTSVYFCARLGYGKFYVMDYWGQGTSVTVSS (SEQ ID NO: 12)
QIQLVQSGPELKKPGASVKISCKASGYTFTNYGMNWVKQAPGQGLKWMGWINTYTGEPTYAQGFTGRFVFS
LDTSVSTAYLQISSLKAEDTSTYFCARLGYGKFYVMDYWGQGTSVTVSS (SEQ ID NO: 13)
QIQLVQSGPELKKPGASVKISCKASGYTFTSYAMNWVKQAPGQGLKWMGWINTYTGEPTYADDFKGRFVFS
LDTSVSTAYLQISSLKAEDTSVYFCARLGYGKFYVMDYWGQGTSVTVSS (SEQ ID NO: 14)
QIQLVQSGHEVKQPGASVKISCKASGYTFTNYGMNWVPQAPGQGLEWMGWINTYTGEPTYADDFKGRFVFS
LDTSASTAYLQISSLKAEDMSMYFCARLGYGKFYVMDYWGQGTSVTVSS (SEQ ID NO: 15)
QIQLVQSGHEVKQPGASVKISCKASGYTFTNYGMNWVPQAPGQGLEWMGWINTYTGEPTYAQGFTGRFVFS
LDTSASTAYLQISSLKAEDMSMYFCARLGYGKFYVMDYWGQGTSVTVSS (SEQ ID NO: 16)
QIQLVQSGHEVKQPGASVKISCKASGYTFTNYGMNWVKQAPGQGLKWMGWINTYTGEPTYADDFTGRFVFS
LDTSASTAYLQISSLKAEDMSMYFCARLGYGKFYVMDYWGQGTSVTVSS (SEQ ID NO: 17)
QIQLVQSGPEVKQPGASVKISCKASGYTFTNYGMNWVPQAPGQGLKWMGWINTYTGEPTYADDFTGRFVFS
LDTSASTAYLQISSLKAEDMSMYFCARLGYGKFYVMDYWGQGTSVTVSS (SEQ ID NO: 18)
QIQLVQSGPEVKQPGASVKISCKASGYTFTNYGMNWVKQAPGQGLKWMGWINTYTGEPTYAQGFTGRFVES
LDTSASTAYLQISSLKAEDMSTYFCARLGYGKFYVMDYWGQGTSVTVSS (SEQ ID NO: 19)
QIQLVQSGPEVKQPGASVKISCKASGYSFTTYGMNWVKQAPGQGLEWMGWINTYTGEPTYADDFKGRFVFS
LDTSASTAYLQISSLKAEDMSTYFCARLGYGKFYVMDYWGQGTSVTVSS (SEQ ID NO: 20)
Light Chain Sequences
DVVMTQTPLSLPVSLGDQASISCRSSQSLVHSNGNTYLHWYLQKPGQSPKLLIYKVSNRFSGVPDRFSGSG
SGTDFTLKISRVEAEDLGVYFCSQSTHVTWTFGGGTKLEIK (SEQ ID NO: 21)
DVVMTQSPLSLPVTLGQPASISCRSSQSLVHSNGNTYLNWYQQRPGQSPRLLIYKVSNRFSGVPDRFSGSG
SGTDFTLKISRVEAEDVGVYFCSQSTHVTWTFGGGTKLEIK (SEQ ID NO: 22)
DVVMTQSPLSLPVTLGQPASISCRSSQSLVHSDGNTYLHWYQQRPGQSPRLLIYKVSNRFSGVPDRFSGSG
SGTDFTLKISRVEAEDVGVYFCSQSTHVTWTFGGGTKLEIK (SEQ ID NO: 23)
DVVMTQSPLSLPVTLGQPASISCRSSQSLVHSNGNTYLHWYQQRPGQSPRLLIYKVSNRDSGVPDRFSGSG
SGTDFTLKISRVEAEDVGVYFCSQSTHVTWTFGGGTKLEIK (SEQ ID NO: 24)
DVVMTQSPLSLPVTLGQPASISCRSSQSLVHSNGNTYLHWFQQRPGQSPRLLIYKVSNRFSGVPDRFSGSG
SGTDFTLKISRVEAEDVGVYFCSQSTHVTWTFGGGTKLEIK (SEQ ID NO: 25)
DIVMTQTPLSLSVTPGQPASISCKSSQSLVHSNGNTYLHWYLQKPGQSPQLLIYKVSNRFSGVPDRFSGSG
SGTDFTLKISRVEAEDVGVYYCSQSTHVTWTFGGGTKLEIK (SEQ ID NO: 26)
DVVMTQSPLSLPVTLGQPASISCRSSQSLVHSNGNTYLHWFQQRPGQSPRRLIYKVSNRFSGVPDRFSGSG
SGTDFTLKISRVEAEDVGVYYCSQSTHVTWTFGGGTKLEIK (SEQ ID NO: 27)
DVVMTQSPLSLPVTLGQPASISCRSSQSLVHSNGNTYLHWFLQRPGQSPRLLIYKVSNRFSGVPDRFSGSG
SGTDFTLKISRVEAEDVGVYYCSQSTHVTWTFGGGTKLEIK (SEQ ID NO: 28)
DVVMTQSPLSLPVTLGQPASISCRSSQSLVHSNGNTYLHWYQQRPGQSPRLLIYKVSNRFSGVPDRFSGSG
SGTDFTLKISRVEAEDVGVYYCSQSTHVTWTFGGGTKLEIK (SEQ ID NO: 29)

In some embodiments of any of the aspects, the antibody, antibody reagent, antigen-binding portion thereof, or CAR further comprises a conservative substitution in a sequence not comprised by a CDR. In some embodiments of any of the aspects, the antibody, antibody reagent, antigen-binding portion thereof, or CAR is fully human or fully humanized. In some embodiments of any of the aspects, the antibody, antibody reagent, antigen-binding portion thereof, or CAR is fully humanized except for the CDR sequences.

In some embodiments of any of the aspects, the reagent or fragment is selected from the group consisting of an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

In one aspect of any of the embodiments of any of the aspects, described herein is a composition comprising the antibody, antibody reagent, antigen-binding portion thereof, or CAR as described herein, and a chemotherapeutic agent. In some embodiments of any of the aspects, wherein the antibody, antibody reagent, or antigen-binding portion thereof is conjugated to the chemotherapeutic agent.

In one aspect of any of the embodiments, described herein is a pharmaceutical composition comprising the antibody, antibody reagent, antigen-binding fragment thereof, CAR, composition, or cell as described herein and a pharmaceutically acceptable carrier.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen (e.g., CHI3L1). Such conservatively modified variants are in addition to and do not exclude polymorphic variants, interspecies homologs, and alleles consistent with the disclosure.

Examples of substitution variants include conservative substitution of amino acids, e.g., in a V_{H} or V_{L}, domain, that do not alter the sequence of a CDR. A conservative substitution in a sequence not comprised by a CDR can be a substitution relative to a wild-type or naturally occurring sequence, e.g., human or murine framework and/or constant regions of an antibody sequence.

A given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (1), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, lIe; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into lIle or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lIle or into Leu.

A variant amino acid or DNA sequence preferably is at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required.

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

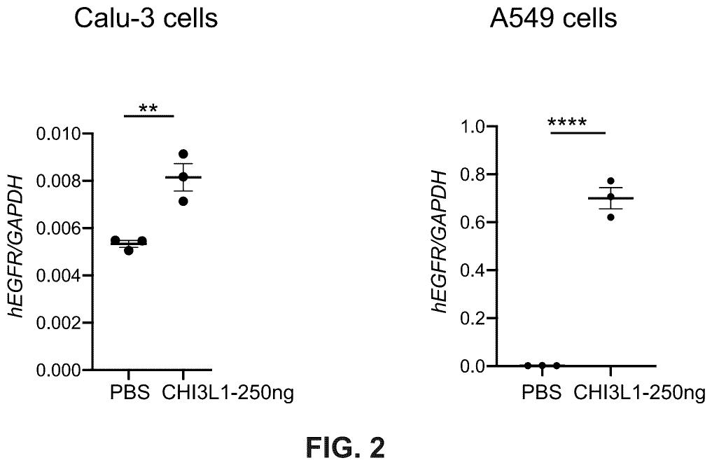

In particular embodiments wherein an antibody, antigen-binding portion thereof, or CAR as described herein comprises at least one CDR which is not identical to the sequence of CHI3L1 CDR provided herein, the amino acid sequence of that at least one CDR can be selected by methods well known to one of skill in the art. For example, Fujii, 2004, “Antibody affinity maturation by random mutagenesis” in Methods in Molecular Biology: Antibody Engineering 248: 345-349 (incorporated by reference herein in its entirety), particularly at FIG. 2 and Section 3.3, describes methods of generating a library for any CDR of interest. This allows one of ordinary skill in the art to identify alternative CDRs, including conservative substitution variants of the specific CDR sequences described herein, which, when present in an antibody or antigen-binding portion thereof as described herein, will result in an antigen or antigen-binding portion thereof which will bind a cancer cell surface antigen. The method described in Fujii et al. also permits one of ordinary skill in the art to screen for a light chain sequence which will give the desired binding behavior when combined with a known heavy chain fragment and vice versa.

In some embodiments, a CAR comprises an extracellular domain comprising an anti-CHI3L1 antibody or antigen-binding portion thereof that binds one or more epitopes of a CHI3L1 polypeptide; a transmembrane domain, one or more intracellular co-stimulatory signaling domains, and a primary signaling domain. Exemplary anti-CHI3L1 antibodies and antigen-binding portions thereof, as well as exemplary epitopes, are described elsewhere herein.

As used herein, “chimeric antigen receptor” or “CAR” refers to an artificially constructed hybrid polypeptide comprising an antigen-binding domain (e.g., an antigen-binding portion of an antibody (e.g., a scFv)), a transmembrane domain, and a T cell signaling and/or T cell activation domain. CARs have the ability to redirect T cell specificity and reactivity toward a selected target in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries), in various embodiments, this scFv is fused to a transmembrane domain and then to an intracellular signaling domain. “First-generation” CARs include those that solely provide CD3zeta (CD3ξ) signals upon antigen binding, “Second-generation” CARs include those that provide both co-stimulation (e.g., CD28 or CD 137) and activation (CD3ξ). “Third-generation” CARs include those that provide multiple costimulatory (e.g., CD28 and CD 137) domains and activation domains (e.g., CD3ξ). In various embodiments, the CAR is selected to have high affinity or avidity for the antigen. Further discussion of CARs can be found in several publications.²⁹

In some embodiments of any of the aspects, a CAR comprises an extracellular binding domain that comprises a humanized CHI3L1-specific binding domain; a transmembrane domain; one or more intracellular co-stimulatory signaling domains; and a primary signaling domain. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., CHI3L1. The binding domain may be derived either from a natural, synthetic, semi-synthetic, or recombinant source.

In some embodiments, the CARs contemplated herein may comprise linker residues between the various domains, e.g., added for appropriate spacing and conformation of the molecule. In particular embodiments the linker is a variable region linking sequence. A “variable region linking sequence,” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, can comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In particular embodiments, the binding domain of the CAR is followed by one or more “spacer domains,” which refers to the region that moves the antigen binding domain away from the effector cell surface to enable proper cell/cell contact, antigen binding and activation. The hinge domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain™. The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8a, CD4, CD28 and CD7, which may be wild-type hinge regions from these molecules or may be altered. In another embodiment, the hinge domain comprises a CD8a hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T cell receptor, CD3e, CD3(, CD4, CD5, CD8a, CD9, CD 16, CD22, CD27, CD28, CD33, CD37, CD45, CD64, CD80, CD86, CD134, CD137, CD152, CD154, and CTLA4.

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Illustrative examples of ITAM containing primary signaling domains that are of particular use in the invention include those derived from TCRζ, FcRγ, FcRβ, CD3γ, CD3δ, CD3ε, CD3ζ, CD22, CD79a, CD79b, and CD66d.

As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In one embodiment, a CAR comprises one or more co-stimulatory signaling domains selected from the group consisting of CD28, CD137, and CD134, and a CD3(primary signaling domain.

In some embodiments, an antibody, or antigen-binding portion thereof, and the chemotherapeutic agent can be present in a scaffold material. Scaffold materials suitable for use in therapeutic compositions are known in the art and can include, but are not limited to, a nanoparticle; a matrix; a hydrogel; and a biomaterial, biocompatible, and/or biodegradable scaffold material. As used herein, the term “nanoparticle” refers to particles that are on the order of about 10⁻⁹ or one to several billionths of a meter. The term “nanoparticle” includes nanospheres; nanorods; nanoshells; and nanoprisms; these nanoparticles may be part of a nanonetwork.

The term “nanoparticles” also encompasses liposomes and lipid particles having the size of a nanoparticle. As used herein, the term “matrix” refers to a 3-dimensional structure comprising the components of a composition described herein (e.g., an antibody or antigen-binding portion thereof). Non-limiting examples of matrix structures include foams; hydrogels; electrospun fibers; gels; fiber mats; sponges; 3-dimensional scaffolds; non-woven mats; woven materials; knit materials; fiber bundles; and fibers and other material formats.³⁰ The structure of the matrix can be selected by one of skill in the art depending upon the intended application of the composition, e.g., electrospun matrices can have greater surface area than foams.

In some embodiments, the scaffold is a hydrogel. As used herein, the term “hydrogel” refers to a three-dimensional polymeric structure that is insoluble in water, but which is capable of absorbing and retaining large quantities of water to form a stable, often soft and pliable, structure. In some embodiments, water can penetrate in between the polymer chains of the polymer network, subsequently causing swelling and the formation of a hydrogel. In general, hydrogels are superabsorbent. Hydrogels have many desirable properties for biomedical applications. For example, they can be made nontoxic and compatible with tissue, and they are highly permeable to water, ions, and small molecules. Hydrogels are super-absorbent (they can contain over 99% water) and can be comprised of natural (e.g., silk) or synthetic polymers, e.g., PEG.

As used herein, “biomaterial” refers to a material that is biocompatible and biodegradable. As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo. As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding amino acid sequence variants of antibodies are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations can be used to construct nucleic acid sequences which encode a monoclonal antibody molecule, antibody reagent, antigen binding region thereof, or CAR.

A nucleic acid molecule, such as DNA, is said to be “capable of expressing” a polypeptide if it contains nucleotide sequences which contain transcriptional and translational regulatory information, and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art.

In some embodiments, a nucleic acid encoding an antibody, antibody reagent, antigen-binding portion thereof, or CAR as described herein is comprised by a vector. In some of the aspects described herein, a nucleic acid sequence encoding an antibody, antibody reagent, antigen-binding portion thereof, or CAR as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody, antigen-binding portion thereof, or CAR as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In one aspect of any of the embodiments, described herein is a cell comprising an antibody, antibody reagent, antigen-binding portion thereof, or CAR as described herein, or a nucleic acid encoding such an antibody, antibody reagent, antigen-binding portion thereof, or CAR.

The expression of an antibody, antibody reagent, antigen-binding portion thereof, or CAR as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence. Moreover, problems associated with retention of initiation codon-derived methionine residues in direct yeast (or bacterial) expression maybe avoided. Any of a series of yeast gene expression systems incorporating promoter and termination elements from the actively expressed genes coding for glycolytic enzymes produced in large quantities when yeast are grown in mediums rich in glucose can be utilized to obtain recombinant antibodies or antigen-binding portions thereof as described herein. Known glycolytic genes can also provide very efficient transcriptional control signals. For example, the promoter and terminator signals of the phosphoglycerate kinase gene can be utilized.

Production of antibodies or antigen-binding portions thereof as described herein in insects can be achieved. For example, by infecting the insect host with a baculovirus engineered to express a transmembrane polypeptide by methods known to those of ordinary skill in the art.

In some embodiments, the introduced nucleotide sequence is incorporated into a plasmid or viral vector capable of autonomous replication in the recipient host. Any of a wide variety of vectors can be employed for this purpose and are known and available to those or ordinary skill in the art. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli., for example. Other gene expression elements useful for the expression of cDNA encoding antibodies, antigen-binding portions thereof, or CARs include, but are not limited to (a) viral transcription promoters and their enhancer elements, such as the SV40 early promoter, Rous sarcoma virus LTR, and Moloney murine leukemia virus; (b) splice regions and polyadenylation sites such as those derived from the SV40 late region, and (c) polyadenylation sites such as in SV40. Immunoglobulin cDNA genes can be expressed, e.g., using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA, the transcriptional promoter can be human cytomegalovirus, the promoter enhancers can be cytomegalovirus and mouse/human immunoglobulin, and mRNA splicing, and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

A gene is assembled in, or inserted into, an expression vector. Recipient cells capable of expressing the chimeric immunoglobulin chain gene product are then transfected singly with an antibody, antigen-binding portion thereof, or CAR, or chimeric H or chimeric L chain-encoding gene or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the genes encoding the antibody, antigen-binding portion thereof, CAR, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the genes in a bacterial system. The genes so produced and amplified in a bacterial host are subsequently used to co-transfect a eukaryotic cell and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively, the genes can be assembled on the same expression vector.

For transfection of the expression vectors and production of the antibodies, antibody reagents, antigen-binding portions thereof, or CARs described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, antigen-binding portion thereof, and/or CAR as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment, as known to one of ordinary skill in the art.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives for hybridoma-based production systems. The main advantages of transgenic animals are potential high yields from renewable sources.

In one aspect, a cell comprising an isolated antibody, antigen-binding portion thereof, or CAR as described herein is provided. In some embodiments, the isolated antibody, antigen-binding portion thereof, or CAR as described herein is expressed on the cell surface. In some embodiments, the cell comprises a nucleic acid encoding an isolated antibody, antigen-binding portion thereof, or CAR as described herein.

In some embodiments, the cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the cell is a T cell; a NK cell; a NKT cell; lymphocytes, such as B cells and T cells; and myeloid cells, such as monocytes, macrophages, eosinophils, mast cells, basophils, and granulocytes.

In particular embodiments, a cell (e.g., an immune cell) is transduced with a retroviral vector, e.g., a lentiviral vector, encoding a CAR. For example, an immune effector cell is transduced with a vector encoding a CAR that comprises an anti-CHI3L1 antibody or antigen binding portion thereof that binds a CHI3L1 polypeptide, with an intracellular signaling domain of CD3(, CD28, 4-1BB, Ox40, or any combinations thereof. Thus, these transduced cells can elicit a CAR-mediated cytotoxic response.

Retroviruses are a common tool for gene delivery. In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to, HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising a CAR to a cell.

Retroviral vectors and more particularly lentiviral vectors may be used in practicing particular embodiments of the present invention. Accordingly, the term “retrovirus” or “retroviral vector”, as used herein is meant to include “lentivirus” and “lentiviral vectors” respectively.

### Kasugamycin (KSM)

In another aspect of any of the embodiments described herein, the methods of the present invention can further include the administration of one or more inhibitor of CHI3L1 and chitinase 1 such as kasugamycin (KSM).

KSM (e.g., a compound of Formula II) is an amino cyclitol glycoside that is isolated from Streptomyces kasugaensis and exhibits antibiotic and fungicidal properties. Like many of the known natural antibiotics, kasugamycin inhibits proliferation of bacteria by tampering with their ability to make new proteins, the ribosome being the major target. Kasugamycin can also be referred to in the art as 2-amino-2-[(2R,3S,5S,6R)-5-amino-2-methyl-6-[(2R,3S,5S,6S)-2,3,4,5,6-pentahydroxycyclohexyl]oxyoxan-3-yl]iminoacetic acid; Kasumin; or 3-O-[2-Amino-4-[(carboxyiminomethyl)amino]-2,3,4,6-tetradeoxy-D-arabino-hexopyranosyl]-D-chiro-inositol.

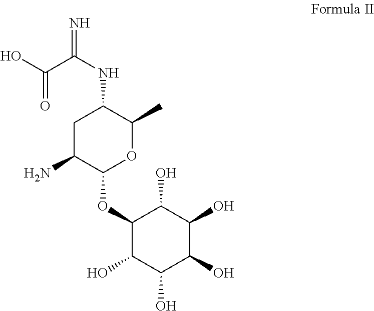 [SMILES: C[C@H]1O[C@H](O[C@H]2[C@H](O)[C@@H](O)[C@@H](O)[C@H](O)[C@@H]2O)[C@@H](N)C[C@@H]1NC(=N)C(=O)O]

Non-limiting examples of KSM derivatives include those described in U.S. Pat. Nos. 3,968,100; 4,554,269; 5,317,095; and 3,480,614.³¹ As used herein, a molecule is said to be a “derivative” of another molecule when it contains additional chemical moieties not normally a part of the molecule and/or when it has been chemically modified. Such moieties can improve the molecule's expression levels, enzymatic activity, solubility, absorption, biological half-life, etc. The moieties can alternatively decrease the toxicity of the molecule, eliminate or attenuate any undesirable side effect of the molecule, etc. Moieties capable of mediating such effects are disclosed in REMINGTON'S PHARMACEUTICAL SCIENCES (1990).³² A “variant” of a molecule is meant to refer to a molecule substantially similar in structure and function to either the entire molecule, or to a fragment thereof. A molecule is said to be “substantially similar” to another molecule if both molecules have substantially similar structures and/or if both molecules possess a similar biological activity. Thus, provided that two molecules possess a similar activity, they are considered variants as that term is used herein even if the structure of one of the molecules not found in the other, or if the structure is not identical. An “analog” of a molecule is meant to refer to a molecule substantially similar in function to either the entire molecule or to a fragment thereof.

We have previously demonstrated in our laboratory that KSM displays unexpected activity in inhibiting the mechanisms of fibrosis, an activity unique to KSM and not displayed by other aminoglycoside antibiotics.³³ We have shown that KSM has the ability to inhibit CHI3L1 and its cousin molecule chitinase 1. It has been reported to have antiviral properties. Based on these observations, it was hypothesized that a therapeutic agent inhibiting CHI3L1 and chitinase 1might be useful in the prevention or treatment of the deleterious effects of Covid-19 infections.

In some embodiments of any of the aspects, an effective dose of a composition comprising KSM or a derivative, analog, or variant thereof as described herein can be administered to a patient once. In some embodiments of any of the aspects, an effective dose of a composition comprising KSM or a derivative, analog, or variant thereof can be administered to a patient repeatedly. For systemic administration, subjects can be administered a therapeutic amount of a composition comprising KSM or a derivative, analog, or variant thereof, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, the KSM or a derivative, analog, or variant thereof as described herein can be administered at a dose of more than about 50 mg/kg. In some embodiments of any of the aspects, the KSM or a derivative, analog, or variant thereof as described herein can be administered at a dose of about 100 mg/kg or greater. In some embodiments of any of the aspects, the KSM or a derivative, analog, or variant thereof as described herein can be administered at a dose from about 50 mg/kg to about 500 mg/kg. In some embodiments of any of the aspects, the KSM or a derivative, analog, or variant thereof as described herein can be administered at a dose from about 50 mg/kg to about 1,000 mg/kg. In some embodiments of any of the aspects, the KSM or a derivative, analog, or variant thereof as described herein can be administered at a dose from about 100 mg/kg to about 500 mg/kg.

In some embodiments of any of the aspects, after an initial treatment regimen, the treatments can be administered on a less frequent basis. For example, after daily treatments for two weeks, treatment biweekly for three months, treatment can be repeated once per month, for six months or a year or longer. Treatment according to the methods described herein can reduce levels of a marker or symptom of a condition, e.g., KSM or a derivative, analog, or variant thereof by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to KSM or a derivative, analog, or variant thereof. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising KSM or a derivative, analog, or variant thereof can be administered over a period of time, such as over a 5 minute, 10 minute, 15 minute, 20 minute, or 25 minute period.

The dosage ranges for the administration of KSM or a derivative, analog, or variant thereof, according to the methods described herein depend upon, e.g., the form of the active ingredient, its potency, and the extent to which symptoms, markers, or indicators of a condition described herein are desired to be reduced, for example the percentage reduction desired for fibrosis. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of KSM or a derivative, analog, or variant thereof in, e.g., the treatment of a condition described herein, or to induce a response as described herein (e.g., a decrease of chitinase activity) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., chitinase activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., collagen levels, degree of fibrosis, and/or BAL cell recovery). It is well within the ability of one skilled in the art to monitor efficacy of administration and/or treatment by measuring any one of such parameters, or any combination of parameters. Efficacy can be assessed in animal models of a condition described herein, for example treatment of mouse models of pulmonary fibrosis. When using an experimental animal model, efficacy of treatment is evidenced when a statistically significant change in a marker is observed, e.g., collagen levels, and/or chitinase activity.

### EGFR Tyrosine Kinase Inhibitors

In one aspect of any of the embodiments described herein, a CHI3L1 inhibitor is administered in combination with at least one EGFR-TKI. EGFR-TKIs useful in the methods of the instant disclosure include, but are not limited to, afatinib (aka Giotrif, Afanix, BIBW 2992); erlotinib (aka Tarceva, Erlotinib hydrochloride); gefitinib (aka Iressa, ZD1839); olmutinib (aka HM-61713, BI-1482694); osimertinib (aka Tagrisso, Nerlynx, AZD9291, mereletinib, osimertinib mesilate, osimertinib mesylate); rociletinib (aka Xegafri, CO-1686, AVL-301), ERBITUX® (aka cetuximab), Vectibix® (aka panitumumab), VISIMPRO® (aka dacomitinib), TYKERB® (aka lapatinib), Potrazza (aka necitumumab), EXKIVITY® (aka mobocertinib), and Caprelsa (aka vandetanib).

### Combinatorial Therapies for EGFR Mutant Nsclc

As indicated above, in one aspect of any of the embodiments described herein, a CHI3L1 inhibitor is administered in combination with at least one EGFR-TKI. In one aspect of any of the embodiments described herein is the combination of a composition comprising an inhibitor of CHI3L1 and a composition comprising an EGFR-TKI (e.g., combined into a single composition or prepared as separate compositions for use or packaging as a paired unit). In some embodiments of any of the aspects, the composition is a pharmaceutical composition. As used herein, the term “pharmaceutical composition” refers to the active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one aspect of any of the embodiments described herein, a CHI3L1 inhibitor is conjugated with at least one EGFR-TKI to form an antibody drug conjugate (ADC). By “conjugated” is meant the covalent linkage of at least two molecules. In some embodiments, the composition can be an antibody-drug conjugate. In particular embodiments, an antibody-drug conjugate comprises an antibody, antibody reagent, or antigen-binding portion thereof as described herein linked to at least one EGFR-TKI. In some embodiments, binding can be non-covalent, e.g., by hydrogen bonding, electrostatic, or van der Waals interactions; however, binding may also be covalent.

In some embodiments, an antibody, antibody reagent, or antigen-binding portion thereof can be bound to and/or conjugated to multiple EGFR-TKIs. In some embodiments, the ratio of a given chemotherapeutic molecule to an antibody or antigen-binding portion thereof can be from about 1:1 to about 1,000:1, e.g., a single antibody reagent molecule can be linked to, conjugated to, etc. from about 1 to about 1,000 individual chemotherapeutic molecules.

### Diagnostic Biomarkers

In some embodiments of any of the aspects described herein, a subject administered a composition described herein can be a subject determined to have an elevated level of CHI3L1 or a level of CHI3L1 that is increased compared to a prior assessment of the level in that subject. In some embodiments of any of the aspects, the elevated level of CHI3L1 is the level of circulating CHI3L1. In some embodiments of any of the aspects described herein, a subject administered a composition described herein can be a subject determined to have cancer cells which are CHI3L1+.

In some embodiments of any of the aspects described herein, the method comprising administering a composition as described herein can further comprise a first step of identifying a subject having an elevated level of CHI3L1. In some embodiments of any of the aspects, the elevated level of CHI3L1 is the level of circulating CHI3L1. In some embodiments of any of the aspects described herein, the method comprising administering a composition as described herein can further comprise a first step of identifying a subject having cancer cells, or a cancer microenvironment, which are CHI3L11+.

As used herein, a “CHI3L1+” cell is a cell expressing an increased level of CHI3L1+, e.g., as compared to a healthy cell of the same type or an average level of CHI3L1 found in healthy cells of the same type. In some embodiments of any of the aspects, an increased level of CHI3L1 can be a level which is at least 1.5× the level found in a reference, e.g., 1.5×, 2×, 3×, 4×, 5×, or greater than the reference level.

In some embodiments of any of the aspects, the expression level of CHI3L1 can be measured by determining the level of an expression product of the CHI3L1 gene, e.g., a CHI3L1 RNA transcript or a CHI3L1 polypeptide. Such molecules can be isolated, derived, or amplified from a biological sample, such as a biofluid. In some embodiments of any of the aspects, a detectable signal is generated by the antibody or antigen-binding portion thereof when a CHI3L1 molecule is present. In some embodiments of any of the aspects, the antibody or antigen-binding portion thereof is detectably labeled or capable of generating a detectable signal. In some embodiments of any of the aspects, the level of the CHI3L1 is determined using a method selected from the group consisting of: Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluorescence assay; mass spectroscopy; FACS; and immunoelectrophoresis assay. In some embodiments of any of the aspects, the antibody or antigen-binding portion thereof is detectably labeled or generates a detectable signal. In some embodiments of any of the aspects, the expression level of CHI3L1 is normalized relative to the expression level of one or more reference genes or reference proteins. In some embodiments of any of the aspects, the reference level of CHI3L1 is the expression level of CHI3L1 in a prior sample obtained from the subject.

In some embodiments of any of the aspects, the level of CHI3L1 can be the level of CHI3L1 polypeptide. Detection of CHI3L1 polypeptides can be according to any method known in the art. Immunological methods to detect CHI3L1 polypeptides in accordance with the present technology include, but are not limited to antibody techniques such as immunohistochemistry, immunocytochemistry, flow cytometry, fluorescence-activated cell sorting (FACS), immunoblotting, radioimmunoassays, western blotting, immunoprecipitation, enzyme-linked immunosorbant assays (ELISA), and derivative techniques that make use of antibody reagents as described herein.

Immunochemical methods require the use of an antibody reagent specific for the target molecule (e.g., the antigen or in the embodiments described herein, a CHI3L1 polypeptide. In some embodiments of any of the aspects, the assays, methods, and/or systems described herein can comprise: an anti-CHI3L1 antibody reagent. In some embodiments of any of the aspects, the antibody reagent can be detectably labeled. In some embodiments of any of the aspects, the antibody reagent can be attached to a solid support (e.g., bound to a solid support). In some embodiments of any of the aspects, the solid support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes.

In one embodiment, an assay, method, and/or system as described herein can comprise an ELISA. In an exemplary embodiment, a first antibody reagent can be immobilized on a solid support (usually a polystyrene micro titer plate). The solid support can be contacted with a sample obtained from a subject, and the antibody reagent will bind (“capture”) antigens for which it is specific (e.g., CHI3L1). The solid support can then be contacted with a second labeled antibody reagent (e.g., a detection antibody reagent). The detection antibody reagent can, e.g., comprise a detectable signal, be covalently linked to an enzyme, or can itself be detected by a secondary antibody which is linked to an enzyme through bio-conjugation. The presence of a signal indicates that both the first antibody reagent immobilized on the support and the second “detection” antibody reagent have bound to an antigen, i.e., the presence of a signal indicated the presence of a CHI3L1 molecule. Between each step the plate is typically washed with a mild detergent solution to remove any proteins or antibodies that are not specifically bound. After the final wash step the plate is developed by adding an enzymatic substrate to produce a visible signal, which indicates the quantity of CHI3L1 polypeptides in the sample. Older ELISAs utilize chromogenic substrates, though newer assays employ fluorogenic substrates with much higher sensitivity. There are other different forms of ELISA, which are well known to those skilled in the art.

In one embodiment, the assays, systems, and methods described herein can comprise a lateral flow immunoassay test (LFIA), also known as the immunochromatographic assay, or strip test to measure or determine the level of CHI3L1 polypeptide in a sample. LFIAs are a simple device intended to detect the presence (or absence) of CHI3L1 in a sample. There are currently many LFIA tests used for medical diagnostics either for home testing, point of care testing, or laboratory use. LFIA tests are a form of immunoassay in which the test sample flows along a solid substrate via capillary action. After the sample is applied to the test strip it encounters a colored antibody reagent which mixes with the sample, and if bound to a portion of the sample, transits the substrate encountering lines or zones which have been pretreated with a second antibody reagent. Depending upon the level of CHI3L1 present in the sample the colored antibody reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water samples etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be used on site in the field. LFIA tests can be operated as either competitive or sandwich assays. Sandwich LFIAs are similar to sandwich ELISA. The sample first encounters colored particles which are labeled with antibody reagents specific for a target (e.g., a CHI3L1-specific antibody reagent). The test line will also contain antibody reagents (e.g., a CHI3L1-specific antibody reagent). The test line will show as a colored band in positive samples. In some embodiments of any of the aspects, the lateral flow immunoassay can be a double antibody sandwich assay, a competitive assay, a quantitative assay or variations thereof. There are a number of variations on lateral flow technology. It is also possible to apply multiple capture zones to create a multiplex test.

A typical test strip consists of the following components: (1) sample application area comprising an absorbent pad (i.e., the matrix or material) onto which the test sample is applied; (2) conjugate or reagent pad—this contains antibody reagent(s) specific to the target which can be conjugated to colored particles (usually colloidal gold particles, or latex microspheres); (3) test results area comprising a reaction membrane—typically a hydrophobic nitrocellulose or cellulose acetate membrane onto which antibody reagents are immobilized in a line across the membrane as a capture zone or test line (a control zone may also be present, containing antibodies specific for the antibody reagents conjugated to the particles or microspheres); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly.

The use of “dip sticks” or LFIA test strips and other solid supports has been described in the art in the context of an immunoassay for a number of antigen biomarkers. U.S. Pat. Nos. 4,943,522; 6,485,982; 6,187,598; 5,770,460; 5,622,871; 6,565,808; 6,809,687; and U.S. Patent Application No. 2004-0184954 A1, which are incorporated herein by reference in their entirety, are non-limiting examples of such lateral flow test devices. U.S. Pat. Nos. 4,444,880; 4,305,924; and 4,135,884, which are incorporated herein by reference in their entirety, describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teaching of these “dip stick” technologies as necessary for the detection of CHI3L1 polypeptides. In some embodiments of any of the aspects, the dip stick (or LFIA) can be suitable for use with urine samples. In some embodiments of any of the aspects, a dip stick can be suitable for use with blood samples.

Immunochemistry is a family of techniques based on the use of a specific antibody, wherein antibodies are used to specifically target molecules inside or on the surface of cells. In some embodiments of any of the aspects, immunohistochemistry (“IHC”) and immunocytochemistry (“ICC”) techniques can be used to detect or measure the levels of CHI3L1 polypeptide. IHC is the application of immunochemistry to tissue sections, whereas ICC is the application of immunochemistry to cells or tissue imprints after they have undergone specific cytological preparations such as, for example, liquid-based preparations. In some instances, signal amplification may be integrated into the particular protocol, wherein a secondary antibody, that includes a label, follows the application of an antibody reagent specific for platelets or leukocytes. Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Buchwalow and Bocker (Eds) “Immunohistochemistry: Basics and Methods” Springer (2010): Lin and Prichard “Handbook of Practical Immunohistochemistry” Springer (2011); which are incorporated by reference herein in their entireties. In some embodiments of any of the aspects, immunocytochemistry may be utilized where, in general, tissue or cells obtained from a subject are fixed by a suitable fixing agent such as alcohol, acetone, and paraformaldehyde, to which is reacted an antibody. Methods of immunocytological staining of human samples is known to those of skill in the art and described, for example, in Burry “Immunocytochemistry: A Practical Guide for Biomedical Research” Springer (2009); which is incorporated by reference herein in its entirety.

In some embodiments of any of the aspects, one or more of the antibody reagents described herein can comprise a detectable label and/or comprise the ability to generate a detectable signal (e.g., by catalyzing a reaction converting a compound to a detectable product). Detectable labels can comprise, for example, a light-absorbing dye, a fluorescent dye, or a radioactive label. Detectable labels, methods of detecting them, and methods of incorporating them into an antibody reagent are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection antibody is labeled with a fluorescent compound. When the fluorescently labeled antibody is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™ rhodamine and derivatives (e.g., Texas red and tetrarhodimine isothiocynate (TRITC)), biotin, phycoerythrin, AMCA, CyDyes™ 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g., Cy3, Cy5 and Cy7 dyes; coumarins, e.g., umbelliferone; benzamide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc.; BODIPY dyes and quinoline dyes.

In some embodiments of any of the aspects, a detectable label can be a radiolabel including, but not limited to ³H, ¹²⁵I, 3S, ¹⁴C, ³²P, and ³³P.

In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments of any of the aspects, a detectable label is a chemiluminescent label, including, but not limited to lucigenin, luminol, luciferin, isoluminol, theromatic acridinium ester, imidazole, acridinium salt and oxalate ester.

In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, antibodies can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e., specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g., from DAKO; Carpinteria, CA.

An antibody reagent can also be detectably labeled using fluorescence emitting metals such as ¹⁵²Eu, or others of the lanthanide series. These metals can be attached to the antibody reagent using such metal chelating groups as diethylenetriaminepentaacetic acid (DTPA) or ethylenediaminetetraacetic acid (EDTA).

The methods as described herein can relate to determining if a subject has an increased level of CHI3L1 relative to a reference level. In some embodiments of any of the aspects, the reference level of CHI3L1 can be the level of CHI3L1 in a healthy subject not having, or not diagnosed as having, e.g., EGFR mutant NSCLC. In some embodiments of any of the aspects, the reference level can be the level in a sample of similar cell type, sample type, sample processing, and/or obtained from a subject of similar age, sex and other demographic parameters as the sample/subject for which the level of CHI3L1 is to be determined. In some embodiments of any of the aspects, the test sample and control reference sample are of the same type, that is, obtained from the same biological source, and comprising the same composition, e.g., the same number and type of cells and/or type of sample material. Accordingly, in some embodiments of any of the aspects, the level of CHI3L1 which is increased can vary as demographic factors such as age, gender, genotype, environmental factors, and individual medical histories vary. In some embodiments of any of the aspects, the reference level can comprise the level of CHI3L1 (e.g., CHI3L1 polypeptide) in a sample of the same type taken from a subject not exhibiting any signs or symptoms of, e.g., cancer. The comparison can also compare (a) the levels in the sample from patients with advanced EGFGR mutant lung cancer and patients with EGFR mutant lung cancer without metastasis and (b) the levels in samples from patients with EGFR mutant lung cancer who are responding to TKI to those that are not or have stopped responding to TKI. In some embodiments of any of the aspects, the reference expression level of CHI3L1 can be the expression level of CHI3L1 in a prior sample obtained from the subject. This permits a direct analysis of any change in levels in that individual.

In some embodiments of any of the aspects, a level of CHI3L1 can be increased relative to a reference level if the level of CHI3L1 is at least 1.25× the reference level, e.g., at least 1.25×, at least 1.5×, at least 2×, at least 3×, at least 4×, at least 5×, at least 6×, or greater of the reference level. In some embodiments of any of the aspects, the expression level of CHI3L1 can be normalized relative to the expression level of one or more reference genes or reference proteins. In some embodiments of any of the aspects, the expression level of CHI3L1 can be normalized relative to a reference value.

In some embodiments of any of the aspects, the expression level of no more than 20 other genes is determined. In some embodiments of any of the aspects, the expression level of no more than 10 other genes is determined.

The term “sample” or “test sample” as used herein denotes a sample taken or isolated from an organism, e.g., a urine sample from a subject. Exemplary biological samples include, but are not limited to, a biofluid sample; serum; plasma; urine; saliva; and/or tumor sample, etc. The term also includes a mixture of the above-mentioned samples. The term “test sample” also includes untreated or pretreated (or pre-processed) biological samples. In some embodiments of any of the aspects, a test sample can comprise cells from a subject. As used herein, the term “biofluid” refers to any fluid obtained from a biological source and includes, but is not limited to, blood, urine, and bodily secretions.

The test sample can be obtained by removing a sample from a subject but can also be accomplished by using a previously isolated sample (e.g., isolated at a prior timepoint and isolated by the same or another person). In addition, the test sample can be freshly collected or a previously collected sample.

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, prepared by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of CHI3L1 as described herein.

In some embodiments of any of the aspects, the methods, assays, and systems described herein can further comprise a step of obtaining a test sample from a subject. In some embodiments of any of the aspects, the subject can be a human subject.

In some embodiments of any of the aspects, the methods, assays, and systems described herein can comprise creating a report based on the level of CHI3L1. In some embodiments of any of the aspects, the report denotes raw values for CHI3L1 in the test sample (plus, optionally, the level of CHI3L1 in a reference sample) or it indicates a percentage or fold increase in CHI3L1 as compared to a reference level, and/or provides a signal that the subject is at risk of having, or not having EGFR mutant NSCLC.

As used herein “at risk of having” refers to at least a 2-fold greater likelihood of having a particular condition as compared to a subject that did not have an elevated and/or increased level of CHI3L1, e.g., a 2-fold, or 2.5-fold, or 3-fold, or 4-fold, or greater risk.

In one aspect, described herein is a kit comprising a composition as described herein, e.g., a composition comprising an antibody, antibody reagent, antigen-binding portion thereof, or CAR as described herein. A kit is any manufacture (e.g., a package or container) comprising at least one reagent, e.g., an antibody, the manufacture being promoted, distributed, or sold as a unit for performing the methods described herein. In some embodiments of any of the aspects, the antibody, antibody reagent, antigen-binding fragment thereof as described herein is immobilized on a solid support. In some embodiments of any of the aspects, the solid support comprises a particle, a bead, a polymer, or a substrate. In some embodiments of any of the aspects, the antibody, antibody reagent or antigen-binding fragment thereof is detectably labeled.

The kits described herein can optionally comprise additional components useful for performing the methods described herein. By way of example, the kit can comprise fluids (e.g., buffers) suitable for composition comprising an antibody, antigen-binding portion thereof, or CAR as described herein, an instructional material which describes performance of a method as described herein, and the like. A kit can further comprise devices and/or reagents for delivery of the composition as described herein. Additionally, the kit may comprise an instruction leaflet and/or may provide information as to the relevance of the obtained results.

### Pharmaceutical Compositions

In one aspect of any of the embodiments, described herein is a composition comprising an antibody, antibody reagent, antigen-binding portion thereof, or CAR as described herein or a nucleic acid encoding an antibody, antibody reagent, antigen-binding portion thereof, or CAR, as described herein, or a cell as described herein. In some embodiments, the composition is a pharmaceutical composition. As used herein, the term “pharmaceutical composition” refers to the active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the invention that will be effective in the treatment of a particular disorder or condition will depend on the nature of the disorder or condition and can be determined by standard clinical techniques.

In some embodiments, the composition comprising an antibody, antibody reagent, antigen-binding portion thereof, or CAR as described herein or a nucleic acid encoding an antibody, antibody reagent, antigen-binding portion thereof, or CAR as described herein can be a lyophilisate.

In some embodiments, the technology described herein relates to a syringe or catheter, including an organ-specific catheter (e.g., renal catheter, biliary catheter, cardiac catheter, etc.), comprising a therapeutically effective amount of a composition described herein.

In one aspect, described herein is a method of inhibiting or killing a CHI3L1+ cell, the method comprising contacting the cell with an isolated antibody, antibody reagent, antigen-binding portion thereof, or CAR as described herein, a nucleic acid encoding such polypeptides, a cell comprising such a polypeptide or nucleic acid, or a composition comprising such a polypeptide or nucleic acid. Inhibiting a CHI3L1+ cell can comprise inhibiting the metabolic activity, metastasis, and/or proliferation of the cell. Assays for measuring metabolic activity, metastasis (e.g., migration assays) and proliferation are well known in the art. Similarly, assays for measuring killing of CHI3L1+ cells, e.g., cell viability assays are well known in the art.

As used herein, a “CHI3L1+” cell is a cell expressing an increased level of CHI3L1+, e.g., as compared to a healthy cell of the same type or an average level of CHI3L1+ found in healthy cells of the same type. In some embodiments, an increased level of CHI3L1 can be a level which is at least 1.5× the level found in a reference, e.g., 1.5×, 2×, 3×, 4×, 5× or greater than the reference level.

In one aspect, the technology described herein relates to a method comprising administering an antibody, antibody reagent, antigen-binding portion thereof, or CAR as described herein or a nucleic acid encoding an antibody, antibody reagent, antigen-binding portion thereof, or CAR as described herein to a subject. In some embodiments, the subject is in need of treatment for a cancer and/or malignancy. In some embodiments, the subject is in need of treatment for: prostate cancer, colon cancer, rectal cancer, ovarian cancer, kidney cancer, breast cancer, glioblastoma, melanoma, malignant melanoma, and lung cancer. In some embodiments, the method is a method of treating a subject. In some embodiments, the method is a method of treating a cancer in a subject.

In one aspect, the technology described herein relates to a method comprising administering an antibody, antibody reagent, antigen-binding portion thereof, or CAR as described herein or a nucleic acid encoding an antibody, antibody reagent, antigen-binding portion thereof, or CAR as described herein to a subject.

In one aspect, described herein is a method of treating cancer in a subject in need thereof, the method comprising administering a cell as described herein, e.g., a cell comprising an antibody, antibody reagent, antigen-binding portion thereof, or CAR as described herein. In some embodiments, the cell is an immune cell.

In one aspect, described herein is a method of treating cancer in a subject in need thereof, the method comprising administering a nucleic acid as described herein or an immune cell comprising the nucleic acid to the subject, wherein the subject's immune cells are caused to express the polypeptide encoded by the nucleic acid. In some embodiments, the immune cell is a T cell. Nucleic acids can be targeted to particular cell types by, e.g., use of a cell-type specific promoter and/or a composition that selectively binds to the desired cell type. For example, conjugation of a nucleic acid to an aptamer can permit targeted delivery.³⁴ In an alternative embodiment, the nucleic acid can be delivered using drug delivery systems such as a nanoparticle, a dendrimer, a polymer, liposomes, or a cationic delivery system. Positively charged cationic delivery systems facilitate binding of a nucleic acid molecule (negatively charged) and also enhance interactions at the negatively charged cell membrane to permit efficient uptake of a nucleic acid by the cell. Cationic lipids, dendrimers, or polymers can either be bound to a nucleic acid or induced to form a vesicle or micelle³⁵ that encases a nucleic acid. The formation of vesicles or micelles further prevents degradation of the nucleic acid when administered systemically. Methods for making and administering cationic-inhibitory nucleic acid complexes are well within the abilities of one skilled in the art. Some non-limiting examples of drug delivery systems useful for systemic delivery of nucleic acids include DOTAP Oligofectamine, “solid nucleic acid lipid particles”, cardiolipin, polyethyleneimine, Arg-Gly-Asp (RGD) peptides, and polyamidoamines. In some embodiments, a nucleic acid forms a complex with cyclodextrin for systemic administration. Methods for administration and pharmaceutical compositions of nucleic acids and cyclodextrins can be found in U.S. Pat. No. 7,427,605. Targeted delivery of nucleic acids is described in several publications.³⁶ By way of example, the nucleic acid can be targeted to immune cells by encapsulating the inhibitor in a liposome comprising ligands of receptors expressed on immune cells, e.g., TCRs. In some embodiments, the liposome can comprise aptamers specific for immune cells.

In some embodiments, the methods described herein relate to CAR-T cell therapy. CAR-T cell and related therapies relate to adoptive cell transfer of immune cells (e.g., T cells) expressing a CAR that binds specifically to a targeted cell type (e.g., cancer cells) to treat a subject. In some embodiments, the cells administered as part of the therapy can be autologous to the subject. In some embodiments, the cells administered as part of the therapy are not autologous to the subject. In some embodiments, the cells are engineered and/or genetically modified to express the CAR. Further discussion of CAR-T therapies can be found in several scientific publications.³⁷

It can generally be stated that a pharmaceutical composition comprising the cells, e.g., T cells or immune cells, described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/mL and generally is greater than 10⁷ cells/mL, generally 10⁸ cells/mL or greater. The clinically relevant number of immune cells can be apportioned into multiple infusions that cumulatively equal or exceed 10⁵, 10⁶, 10⁷, 10⁸, 109, 10¹⁰, 10¹¹, or 10¹² cells. In some aspects of the present invention, particularly since all the infused cells will be redirected to a particular target antigen, lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1a, etc.) as described herein to enhance induction of the immune response. In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. In some embodiments, one dose of cells can be administered. In some embodiments, the dose of cells can be repeated, e.g., once, twice, or more. In some embodiments, the dose of cells can be administered on, e.g., a daily, weekly, or monthly basis.

The dosage ranges for the agent depend upon the potency and encompass amounts large enough to produce the desired effect, e.g., slowing of tumor growth or a reduction in tumor size. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In some embodiments, the doses are given once a day, or multiple times a day, for example but not limited to three times a day. In some embodiments, the doses recited above are administered daily for several weeks or months. The duration of treatment depends upon the subject's clinical progress and responsiveness to therapy.

In some embodiments, the dose can be from about 2 mg/kg to about 15 mg/kg. In some embodiments, the dose can be about 2 mg/kg. In some embodiments, the dose can be about 4 mg/kg. In some embodiments, the dose can be about 5 mg/kg. In some embodiments, the dose can be about 6 mg/kg. In some embodiments, the dose can be about 8 mg/kg. In some embodiments, the dose can be about 10 mg/kg. In some embodiments, the dose can be about 15 mg/kg. In some embodiments, the dose can be from about 100 mg/m² to about 700 mg/m². In some embodiments, the dose can be about 250 mg/m². In some embodiments, the dose can be about 375 mg/m². In some embodiments, the dose can be about 400 mg/m2. In some embodiments, the dose can be about 500 mg/m².

In some embodiments, the dose can be administered intravenously. In some embodiments, the intravenous administration can be an infusion occurring over a period of from about 10 minutes to about 3 hours. In some embodiments, the intravenous administration can be an infusion occurring over a period of from about 30 minutes to about 90 minutes.

In some embodiments the dose can be administered about weekly. In some embodiments, the dose can be administered weekly. In some embodiments, the dose can be administered weekly for from about 12 weeks to about 18 weeks. In some embodiments, the dose can be administered about every two weeks. In some embodiments the dose can be administered about every three weeks. In some embodiments, the dose can be from about 2 mg/kg to about 15 mg/kg administered about every two weeks. In some embodiments, the dose can be from about 2 mg/kg to about 15 mg/kg administered about every three weeks. In some embodiments, the dose can be from about 2 mg/kg to about 15 mg/kg administered intravenously about every two weeks. In some embodiments, the dose can be from about 2 mg/kg to about 15 mg/kg administered intravenously about every three weeks. In some embodiments, the dose can be from about 200 mg/m² to about 400 mg/m² administered intravenously about every week. In some embodiments, the dose can be from about 200 mg/m2 to about 400 mg/m² administered intravenously about every two weeks. In some embodiments, the dose can be from about 200 mg/m² to about 400 mg/m² administered intravenously about every three weeks. In some embodiments, a total of from about 2 to about 10 doses are administered. In some embodiments, a total of four doses are administered. In some embodiments, a total of five doses are administered. In some embodiments, a total of six doses are administered. In some embodiments, a total of seven doses are administered. In some embodiments, a total of eight doses are administered. In some embodiments, the administration occurs for a total of from about four weeks to about 12 weeks. In some embodiments, the administration occurs for a total of about six weeks. In some embodiments, the administration occurs for a total of about eight weeks. In some embodiments, the administration occurs for a total of about 12 weeks. In some embodiments, the initial dose can be from about 1.5 to about 2.5 fold greater than subsequent doses.

In some embodiments, the dose can be from about 1 mg to about 2000 mg. In some embodiments, the dose can be about 3 mg. In some embodiments, the dose can be about 10 mg. In some embodiments, the dose can be about 30 mg. In some embodiments, the dose can be about 1000 mg. In some embodiments, the dose can be about 2000 mg. In some embodiments, the dose can be about 3 mg given by intravenous infusion daily. In some embodiments, the dose can be about 10 mg given by intravenous infusion daily. In some embodiments, the dose can be about 30 mg given by intravenous infusion three times per week.

A therapeutically effective amount is an amount of an agent that is sufficient to produce a statistically significant, measurable change in tumor size, tumor growth etc. (efficacy measurements are described below herein). Such effective amounts can be gauged in clinical trials as well as animal studies.

An agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. It is preferred that the compounds used herein are administered orally, intravenously or intramuscularly to a patient having cancer. Local administration directly to a tumor mass is also specifically contemplated.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In some embodiments, the methods further comprise administering the pharmaceutical composition described herein along with one or more additional chemotherapeutic agents, biologics, drugs, or treatments as part of a combinatorial therapy. In some such embodiments, the chemotherapeutic agent biologic, drug, or treatment is selected from the group consisting of radiation therapy, surgery, antibody reagents, and/or small molecules.

In some embodiments of the methods described herein, the methods further comprise administering one or more chemotherapeutic agents to the subject being administered the pharmaceutical composition described herein. Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1 I and calicheamicin omegal1; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any one of the above.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins, such as small molecule toxins or enzymatically active toxins of bacterial, fungal, plant or animal origin, including fragments and/or variants thereof.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most, if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use.³⁸ The CHI3L1 inhibitors described herein can be used in conjunction with additional chemotherapeutic agents.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In some embodiments, the methods described herein can further comprise administering an additional immunotherapy to the subject. As used herein, “immunotherapy” refers to a diverse set of therapeutic strategies designed to induce the patient's own immune system to fight the tumor, and include, but are not limited to, immunotherapy, vaccines to generate specific immune responses, administration of cytokines, growth factors and/or signaling molecules that stimulate one or more immune cell type (e.g., interleukins), ex vivo expansion and/or stimulation of lymphocytes and/or dendritic cell specific for a tumor antigen prior to reintroduction to the patient, imiquimod, adoptive cell transfer, and/or the methods described, e.g., in International Patent Publication WO 2003/063792 and U.S. Pat. No. 8,329,660. In some embodiments, the immunotherapy stimulates NK responses. In other embodiments, the immunotherapy is an adoptive cell transfer approach, i.e., adoptive immunotherapy.

The efficacy of a given treatment for, e.g., cancer, can be determined by the skilled clinician. However, a treatment is considered “effective treatment,” as the term is used herein, if any one or all of the signs or symptoms of e.g., a tumor are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

An effective amount for the treatment of a disease means that amount which, when administered to a mammal in need thereof, is sufficient to result in effective treatment as that term is defined herein, for that disease. Efficacy of an agent can be determined by assessing physical indicators of, for example cancer, e.g., tumor size, tumor mass, tumor density, angiogenesis, tumor growth rate, etc.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Materials and Methods

### Cell Lines and Cell Culture

Calu-3 (HTB-55) and A549 (CCL-185) lung epithelial cells were purchased from American Tissue Type Collection (ATCC) and maintained at 37 °C in Dulbecco's modified eagle medium (DMEM) supplemented with high glucose, L-Glutamine, minimal essential media (MEM) non-essential amino acids, penicillin/streptomycin and 10% fetal bovine serum (FBS) until used. The Calu-3 cells were authenticated through STR profiling and mycoplasma testing by ATCC. EGFR mutant cell lines H1975 (CRL-5908), HCC827 (CRL-2868) were purchased from (ATCC) and maintained as recommended by the company.

### RNA Extraction and Real-Time PCR

Total cellular RNA was obtained using TRlzol reagent (ThermoFisher Scientific) followed by RNA extraction using RNeasy Mini Kit (Qiagen, Germantown, MD) according to the manufacturer's instructions. mRNA was measured and used for real time (RT)-PCR, as described previously.^{39,40} The primer sequences used in these studies are summarized in Table 4. Ct values of the test genes were normalized to internal housekeeping genes such as β-actin, GAPDH or RPL13a.

TABLE 4
Primer pairs used for qRT-PCR

| Genes | Sense (5′- 3′) | Anti-sense (5′-3′) |
|---|---|---|
| CHI3L1 | GTGAAGGCGTCTCAAACAGG | GAAGCGGTCAAGGGCATCT |
| EGF | TTGGTGATGGGAGGATGACT | GGCCAGTGACTCAGCAGAAA |
| EGFR | AACACCCTGGTCTGGAAGTA | TCGTTGGACAGCCTTCAAGA |
| | CG | CC |
| TGF-α | CTCAGACACATGCCTACACC | GAACATCTCTGCTCGTCGCT |
| | T | |
| TAZ | AAACCCCTTGGAAATAGTCA | GGAAAGCCAAATCCCAGATG |
| | GC | AG |
| TEAD | GCCTCCGAGAGCTATATGAT | TCACTCCGTAGAAGCCACCA |
| | CG | |
| SOX2 | ACCAGCGCATGGACAGTTAC | CGAGCTGGTCATGGAGTTGT |
| SOX9 | AAGTCGGTGAAGAACGGGCA | TGTGTAGACGGGTTGTTCCC |
| YAP-1 | ACCCACAGCTCAGCATCTTC | GGTTCATGGCAAAACGAGGG |

### Generation of Monoclonal Antibodies Against CHI3L1 (FRG)

The murine monoclonal anti-CHI3L1 antibody (FRG) was generated using peptide antigen (amino acid 223-234 of human CHI3L1) as immunogen through Abmart Inc (Berkeley Heights, NJ). This monoclonal antibody specifically detects both human and mouse CHI3L1 with high affinity (K_{d}≈1.1×10⁻⁹). HEK-293T cells were transfected with the FRG construct using Lipofectamine™ 3000 (Invitrogen, #L3000015). Supernatant was collected for 7 days, and the antibody was purified using a protein A column (ThermoFisher scientific, #89960). Ligand binding affinity and sensitivity were assessed using ELISA techniques.

### Melanoma Lung Metastasis and Antibody Treatment

B16-OVA M04 cell line is a model for cancer immunotherapy, expressing ovalbumin (OVA) in order to facilitate strong immune responses to tumor antigens. B16F10 (ATCC® CCL-6475™) is a murine melanoma cell line from a C57BL/6J mouse. It is a subclone of the B16 tumor line, generated by injecting mice with B16 tumor cells, collecting and culturing secondary tumor growths, and injecting them into fresh mice, a total of 10 times.

B16-F10, a mouse melanoma cell line, was purchased from ATCC (Cat #: CRL-6475, Manassas, VA) and maintained in Dulbecco's Modified Eagles Medium (DMEM) supplemented with 10% Fetal bovine Serum (FBS) and 1% penicillin & streptomycin as recommended. As previously described, intraperitoneal injection of the noted doses of anti-CHI3L1 (FRG) alone or in combination with EGFR Tyrosine Kinase Inhibitors (TKIs) gefitinib, erlotinib or osimertinib (Selleckchem) were started on the day of the B16 tumor cell challenge and continued every other day for 2 weeks. Metastasis was assessed and quantified by counting the melanoma colonies (black dots) on the pleural surface as previously described.^{41,42}

### Immunofluorescence Assay (Immunocytochemistry)

H1975 or HCC827 cells were cultured in 4 well chamber slides (10⁶ cell/well) with and without anti-CHI3L1 antibody (FRG) alone or with TKIs (gefitinib and osimertinib). The cells were fixed on the slides, permeabilized, treated with blocking buffer, and then incubated with fluorescent-labelled detecting antibodies, as indicated in each figure description. The photographs of cellular immunofluorescence were taken with fluorescent microscopes (Nikon, Eclipse Ti2).

Terminal Deoxynucleotidyl Transferase dUTP Nick End Labeling (TUNEL) Staining

Terminal deoxynucleotidyl transferase mediated dUTP nick end labeling (Tunel) for the in situ assessment of cell death was performed as follows. H1975 cells were cultured in 4 well chamber slides (10⁶ cell/well) with and without anti-CHI3L1 antibody (FRG) alone or with TKIs (gefitinib and osimertinib). The cells were fixed on the slides, permeabilized, treated with blocking buffer, and then incubated with fluorescent-labelled enzyme solution (In Situ Cell Death Detection Kit, Fluorescein (Roche-11684795910) for 1 hour, as indicated in each figure description. The photographs of cellular immunofluorescence were taken with fluorescent microscopes (Nikon, Eclipse Ti2).

### Example 1 TGF-α and CHI3L1 Stimulation of CHI3L1 (CHI3L1 Autoinduction and EGFR in Cells with Wild-Type EGFR

Studies were undertaken to define the effects of TGF-α and CHI3L1 on the expression of CHI3L1 (CHI3L1 autoinduction) and epidermal growth factor receptor (EGFR). In these experiments, Calu3 cells, which have a wild type (WT) EGFR, were used and incubated with TGF-α (25 nM and 100 nM) or CHI3L1 (250 ng/mL, 500 ng/mL, and 1.0 g/mL) for 72 hours.

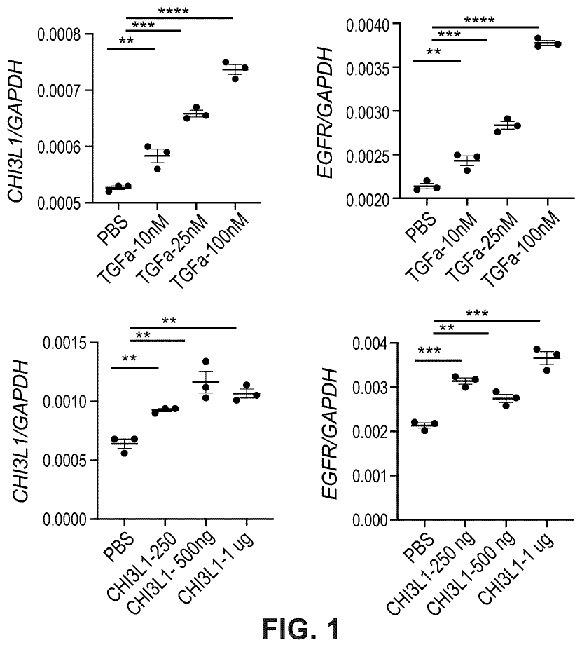

As shown in the top panels FIG. 1, 10 nM (P<0.01), 25 nM (P<0.001) and 100 nM (P<0.00001) TGF-α significantly stimulated CHI3L1 and EGFR in cells with wild-type EGFR compared to the PBS control. As shown in the bottom panels FIG. 1, 250 ng/mL, 500 ng/mL, and 1.0 g/mL CHI3L1 significantly stimulated CHI3L1 autoinduction (P<0.01, P<0.01, P<0.01, respectively) and EGFR (P<0.001, P<0.01, P<0.001, respectively) in cells with wild-type EGFR compared to the PBS control.

### Example 2 CHI3L1 Stimulation of the Expression of EGFR in Cells with Wild-Type EGFR

Studies were undertaken to define the effects of CHI3L1 on the expression of epidermal growth factor receptor (EGFR) in two types cells with a wild type (WT) EGFR, Calu 3 cells and A549 epithelial cells. Cells were incubated with CHI3L1 for 72 hours. Gene expression was evaluated by RT-PCR.

As shown in FIG. 2, 250 ng/mL CHI3L1 significantly stimulated the expression of EGFR in both types of cells with wild-type EGFR: Calu 3 cells (P<0.01) and A549 epithelial cells (P<0.0001).

### Example 3 CHI3L1 Stimulates CHI3L1 Autoinduction and Components of EGFR Axis in Cells with “Common” EGFR Mutations

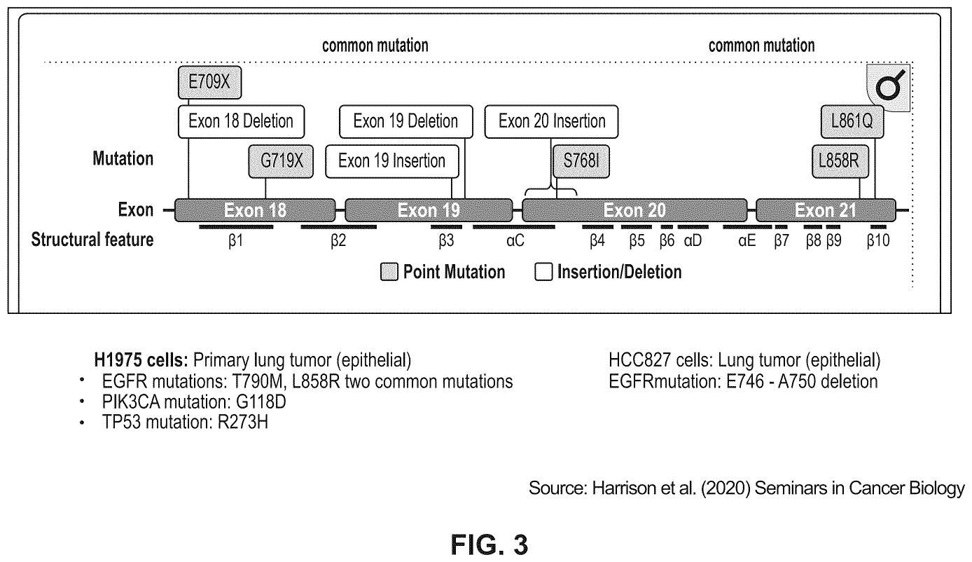

There are a number of EGFR mutations that play a role in the pathogenesis of EGFR mutant NSCLC; some are common, and some are not. The common ones include L858R and mutations of exon 19 and these are schematically illustrated in FIG. 3.

Studies were undertaken to define the effects of CHI3L1 on the expression of CHI3L1 (CHI3L1 autoinduction), epidermal growth factor receptor (EGFR) and its physiologic ligands EGF and TGF-α. In these experiments, H1975 cells, which have a mutant EGFR, were incubated with CHI3L1 for 72 hours. Gene expression was evaluated by RT-PCR.

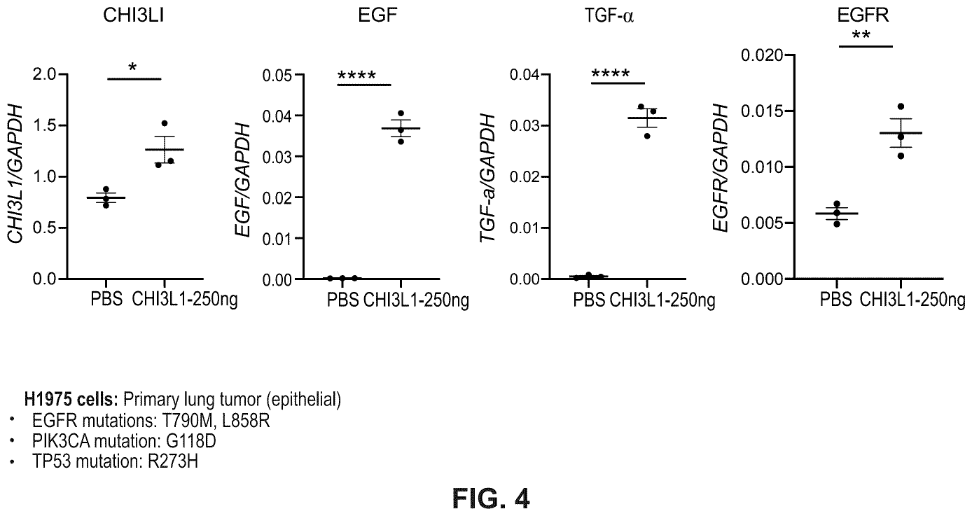

As shown in FIG. 4, 250 ng/mL CHI3L1 significantly stimulated the expression of CHI3L1 autoinduction (P<0.05), as well as the expression of EGFR (P<0.01) and its physiologic ligands EGF (P<0.00001) and TGF-α (P<0.00001).

### Example 4 Dose Dependent Effects of TGF-α and CHI3L1 on CHI3L1 Autoinduction And EGFR in Cells with EGFR Mutations

Studies were undertaken to define the dose response characteristics of the effects of CHI3L1 or TGF-α on the expression of CHI3L1 (CHI3L1 autoinduction) or EGFR. In these experiments, H1975 cells, which have a mutant EGFR, were incubated with CHI3L1 (250 ng/mL, 500 ng/mL, and 1.0 g/mL) or TGF-α (10 nM, 25 nM, and 100 nM) for 72 hours. Gene expression was evaluated by RT-PCR.

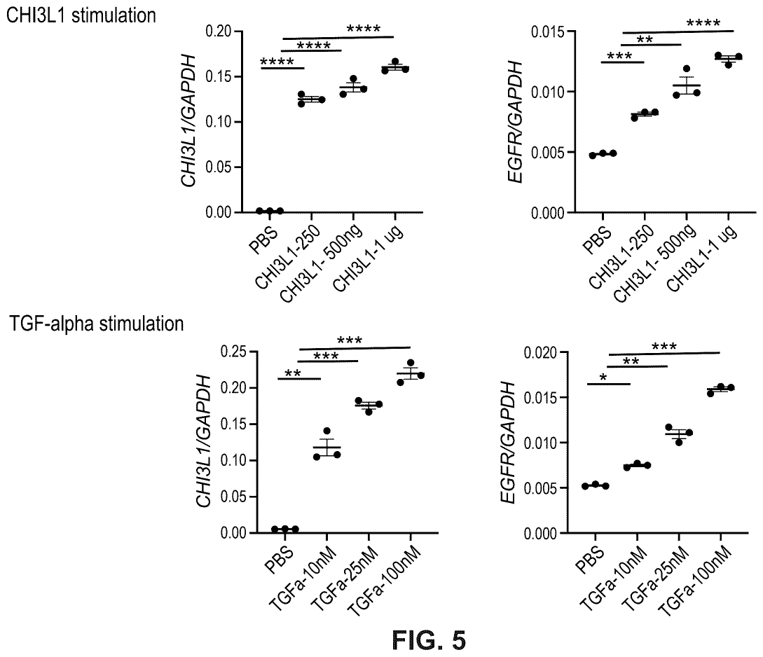

As shown in FIG. 5, the effects of CHI3L1 (top panels) and TGF-α (bottom panels) on CHI3L1 autoinduction (left panels) and EGFR in cells with EGFR mutations (right panels) were statistically significant and dose dependent.

Example 5 CHI3L1 Stimulation of EGFR in Cells with Other EGFR Mutations and Inhibition by an Anti-CHI3L1 Antibody

Studies were undertaken to define the effects of CHI3L1 on the expression of EGFR and the ability of monoclonal anti-CHI3L1 antibody (FRG) to block these effects. In these experiments, HCC827 cells, which have a mutant EGFR, were incubated with CHI3L1 for 72 hours. Gene expression was evaluated by RT-PCR.

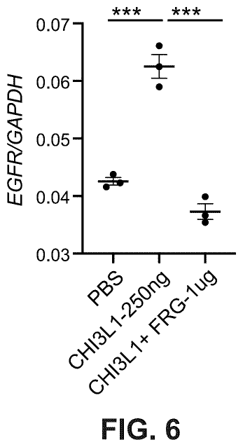

As shown in FIG. 6, 250 ng/mL CHI3L1 significantly increased the expression of EGFR (P<0.001) and this CHI3L1-induced stimulation was inhibited by 1.0 g FRG, a monoclonal anti-CHI3L1 antibody (P<0.001).

Example 6 Exaggerated Baseline Accumulation of CHI3L1 and EGFR In Epithelial Cells with “Common” EGFR Mutations

Double label immunocytochemistry (ICC) was used to define the accumulation and location of CHI3L1 and EGFR in HCC827 which have mutant EGFR. DAPI was used to stain nuclei.

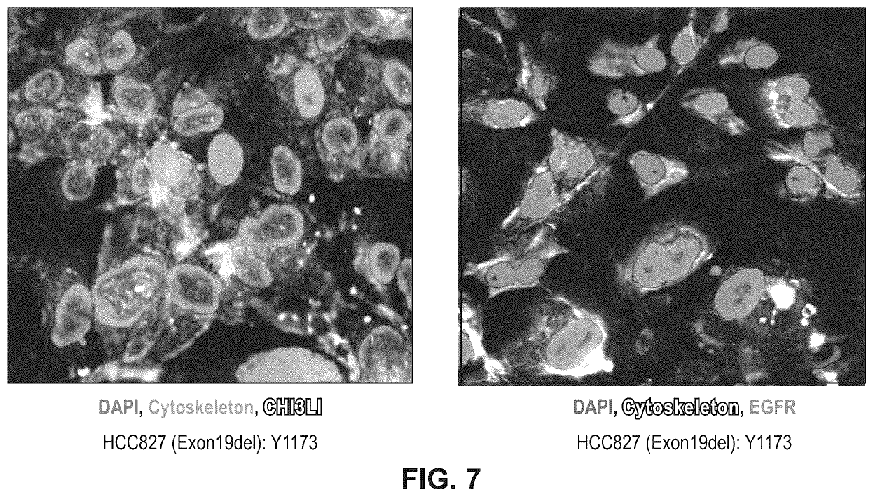

As shown in FIG. 7, exaggerated levels of CHI3L1 (red) were seen in nuclei of these cells and exaggerated levels of EGFR (green) were seen in their cytoplasm. EXAMPLE 7 GEFITINIB INHIBITION OF THE MURINE PULMONARY ACCUMULATION OF PLEURAL COLONIES OF B16-F10 MALIGNANT MELANOMA CELLS AND BAL CHI3L1

The B16-F10 metastasis model was used to define the effects of the tyrosine kinase inhibitor (TKI), gefitinib, on the pulmonary spread of malignant melanoma and the accumulation of bronchoalveolar lavage (BAL) CHI3L1 during these circumstances. In these experiments, B16-F10 malignant melanoma cells were injected into wild type mice. The ability of the cells to spread to the lung (manifest as black pleural lesions) was evaluated visually and quantitatively two weeks later. These experiments compared mice that were administered gefitinib versus its DMSO vehicle control.

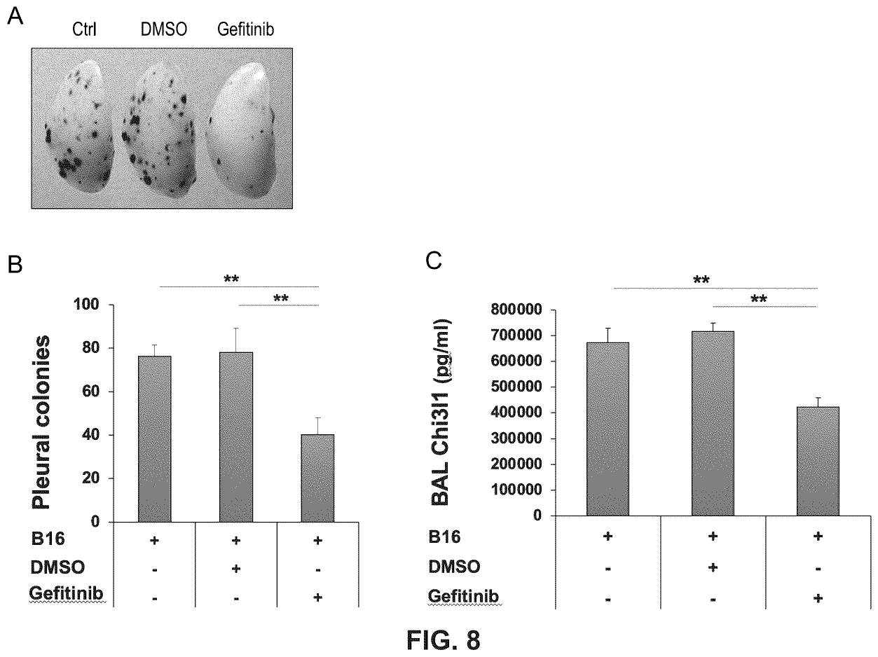

As shown in FIG. 8A, gefitinib inhibited the murine pulmonary accumulation of pleural colonies of B16-F10 malignant melanoma cells. FIG. 8B, which provides the quantitation of pleural colony numbers, indicates that gefitinib significantly reduced the number of pleural colonies (P<0.01) and supporting its function as a potent inhibitor of metastasis. FIG. 8C, which provides the BAL levels of CHI3L1 assessed by ELISA assay, indicates that gefitinib significantly reduced the accumulation of CHI3L1 (P<0.01) in bronchoalveolar lavage and supporting its function as a potent inhibitor of the production of CHI3L1.

### Example 8 Erlotinib Inhibition of the Murine Pulmonary Accumulation of Pleural Colonies of B16-F10 Malignant Melanoma Cells

The B16-F10 metastasis model was used to define the effects of the tyrosine kinase inhibitor (TKI) erlotinib, or its DMSO vehicle control, on the pulmonary spread of malignant melanoma in WT mice (Tg−) and mice in which CHI3L1 is overexpressed in the lung in a transgenic manner (Tg+). In these experiments, B16-F10 malignant melanoma cells were injected into wild type mice or Tg mice. The ability of the cells to spread to the lung (manifest as black pleural lesions) was evaluated quantitatively two weeks later.

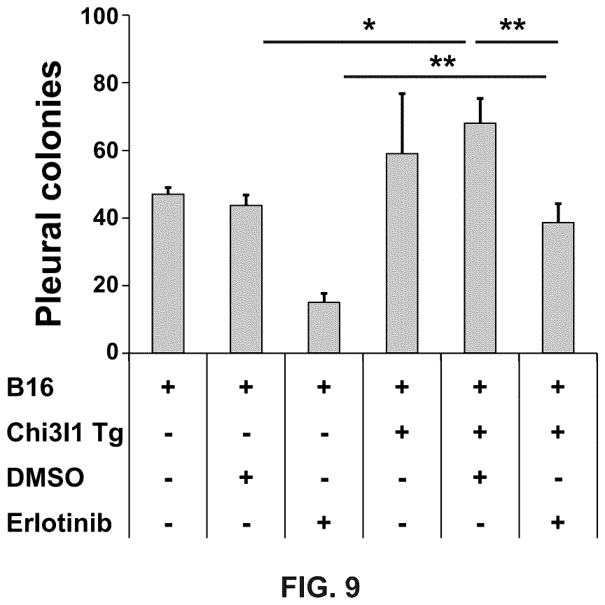

As shown in FIG. 9, the transgenic overexpression of CHI3L1 increased B16-F10 metastasis (P<0.05) and erlotinib inhibited B16-F10 cell metastasis even when CHI3L1 was overexpressed in the lung (P<0.01).

Example 9 CHI3L1 Inhibitor and Osimertinib Inhibit the Accumulation of Pleural Colonies of B16-F10 Cells

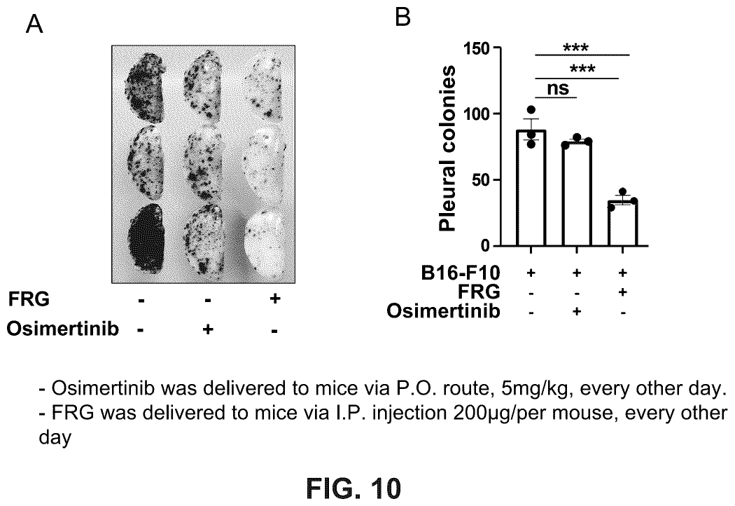

Studies were undertaken to compare the effects of osimertinib and the anti-CHI3L1 antibody, FRG, on B10-F10 metastasis. B16-F10 cells were injected by tail vein and mice were treated every other day with 5 mg/kg osimertinib or 200 g/mouse FRG. Metastasis were quantitated 14 days later. FIG. 10A shows mice lungs with metastasis, while FIG. 10B provides the quantitation of pleural colony numbers.

As can be seen in FIG. 10A, both the anti-CHI3L1 antibody, FRG, and the TKI, osimertinib, inhibited the accumulation of pleural colonies of B16-F10 cells visually. FIG. 10B demonstrates that FRG induced a statistically significant reduction of pleural colony numbers (P<0.01).

### Example 10 Anti-CHI3L1 Antibody Inhibits CHI13L1 Autoinduction and CHI13L1 Stimulation of Components of the EGFR Axis in Cells with “Common” EGFR Mutations

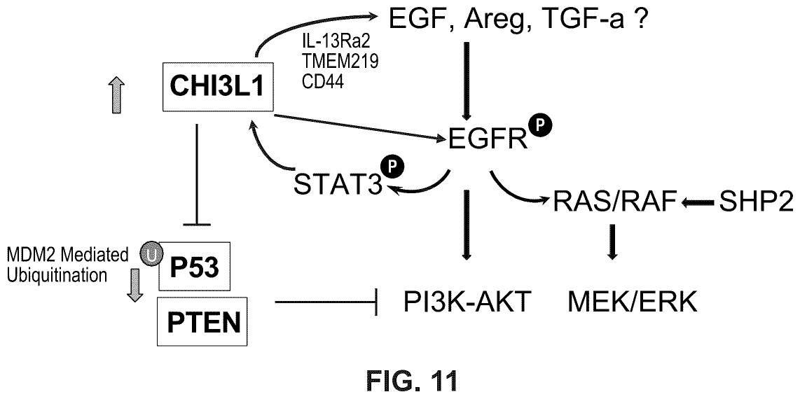

FIG. 11 schematically illustrates the contributions of CHI3L1 to the EGFR axis. EGFR activation stimulates CHI3L1 via Stat 3. The CHI3L1 then feeds back to stimulate the expression and accumulation of EGFR and its physiologic ligands (EGF, AREG, TGF-α). CHI3L1 also feeds back to inhibit the tumor suppressors P53 and PTEN, which in turn inhibit P13K and MAPK activation.

Studies were undertaken to define the effects of CHI3L1 on the expression of CHI3L1 (CHI3L1 autoinduction), epidermal growth factor receptor (EGFR) and its physiologic ligands EGFR and TGF-α. In these experiments, the effects of the anti-CHI3L1 antibody, FRG, was characterized on these inductive events. HCC827 cells, which have a mutant EGFR, were incubated with recombinant (r) CHI3L1 for 72 hours. Gene expression was evaluated by RT-PCR.

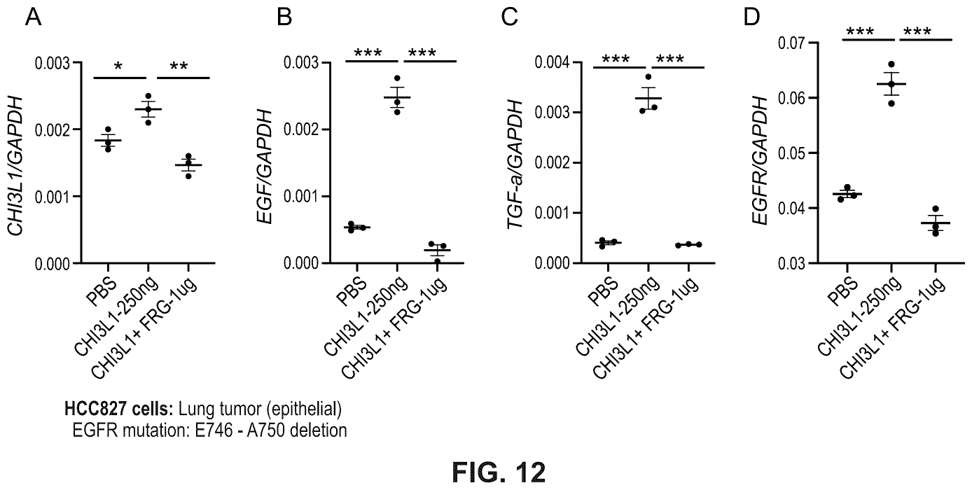

As shown in FIG. 12, 250 ng/mL CHI3L1 significantly stimulated the expression of CHI3L1 autoinduction (FIG. 12A, P<0.05), as well as the expression of EGFR (FIG. 12D P<0.001) and its physiologic ligands EGF (P<0.001) and TGF-α (P<0.001). Importantly, FRG significantly inhibited the CHI3L1 autoinduction (FIG. 12A, P<0.01), as well as the CHI3L1-induced expression of EGFR (FIG. 12D P<0.001) and its physiologic ligands EGF (P<0.001) and TGF-α (P<0.001).

These data demonstrate that CHI3L1 is a potent stimulator of CHI3L1 autoinduction and EGFR, and its physiologic ligands TGF-α and EGF and that FRG is a potent inhibitor of these inductive events.

### Example 11 Effects of Anti-CHI3L1 and Osimertinib, Alone and in Combination, on the EGFR Axis in Cells with “Common” EGFR Mutations

Studies were undertaken to define potential synergistic effects between CHI3L1 inhibitors and TKIs by investigating the effects of the anti-CHI3L1 antibody FRG (200 g/mouse) and the TKI osimertinib (5 mg/kg osimertinib), alone and in combination, on the expression of CHI3L1, EGFR, and EGF in HCC827 cells.

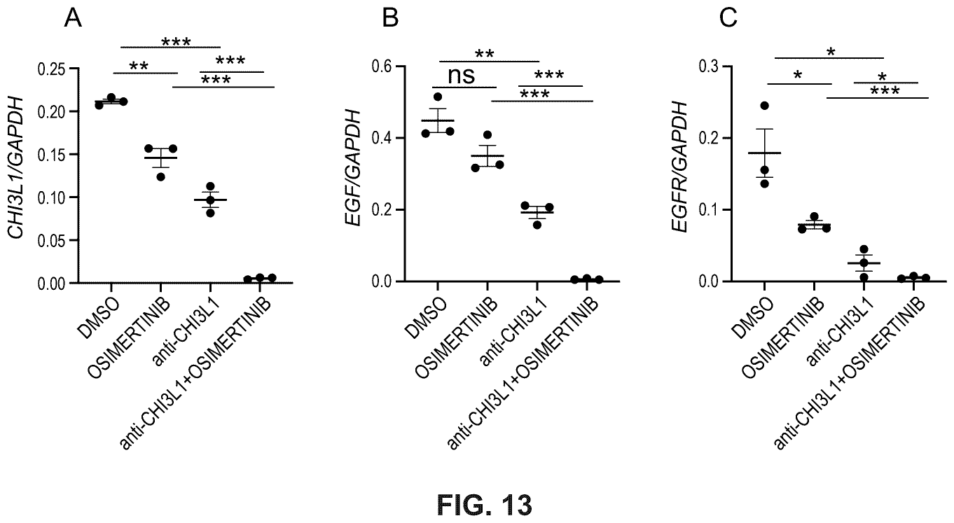

As shown in FIG. 13, although FRG and osimertinib individually were able to reduce the expression of CHI3L1 (FIG. 13A), EGFR (FIG. 13C), and EGF (FIG. 13B) in HCC827 cells, osimertinib and anti-CHI3L1 (FRG) in combination worked synergistically and were more potent than each individually.

### Example 12 Combinatorial Effects of Low Doses of Anti-CHI3L1 (Frg) and Tki (Osimertinib) on CHI3L1 and EGFR Axis in Cells with “Common” EGFR Mutations

In order to investigate the synergistic effects further, studies were undertaken to assess the effects of low doses of anti-CHI3L1 FRG (5 ng) and the TKI osimertinib (0.125, 0.5, 1.0 ng), alone and in combination, on the expression of CHI3L1 and EGFR in H1975 cells with “common” EGFR mutations.

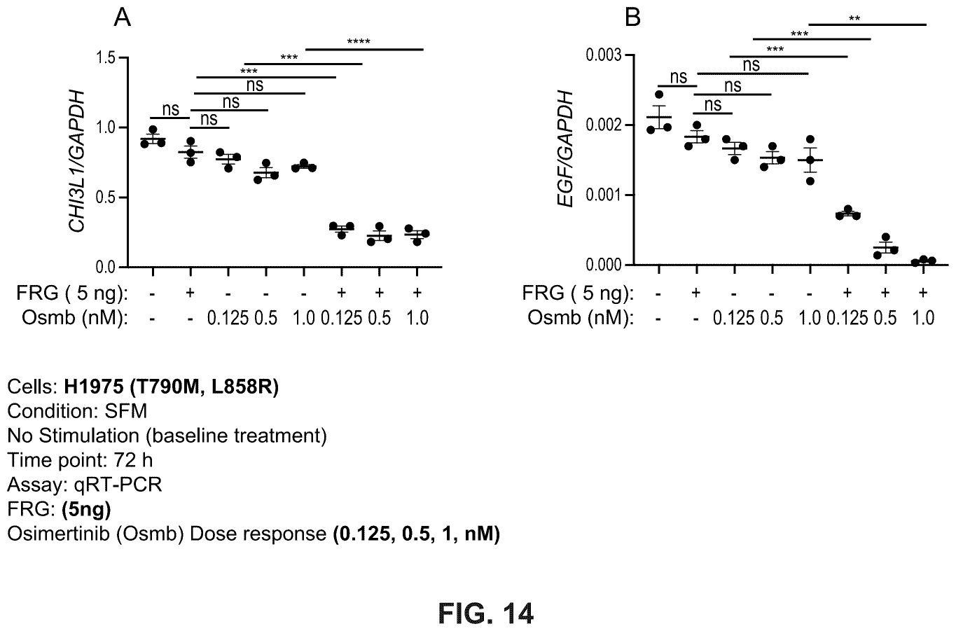

As shown in FIG. 14, the low doses of FRG (5 ng) and osimertinib (0.125, 0.5, 1.0 ng), when administered alone, did not significantly affect the expression of CHI3L1 and EGFR. However, when used in combination, significant inhibition in CHI3L1 expression (FIG. 14A) were noted: 5 ng/mL FRG with Osimertinib (Osmb) at 0.125 nM (P<0.001), 0.5 nM (P<0.001), and 1.0 nM (P<0.0001). Similarly, when used in combination, significant inhibition in EGFR expression (FIG. 14B) were noted: 5 ng/mL FRG with osimertinib at 0.125 nM (P<0.001), 0.5 nM (P<0.001), and 1.0 nM (P<0.01).

These data confirm that osimertinib and FRG in combination interact in a synergistic manner to inhibit the expression of CHI3L1 (FIG. 14A) and EGFR (FIG. 14B), and that these inhibitory effects are more potent than the effects of each moiety individually in cells with “common” EGFR mutations.

### Example 13 Effects of Anti-CHI3L1 (Frg) and Tki (Gefitinib), Alone and in Combination, on the Survival of H1975 Cells with “Common” EGFR Mutations

Studies were undertaken to define the effects of the TKI gefitinib and the anti-CHI3L1 FRG, alone and in combination, on DNA injury and cell death in EGF stimulated H1975 cells using terminal deoxynucleotidyl transferase dUTP nick end labeling (TUNEL) analysis. TUNEL staining is a method for detecting DNA fragmentation by labelling the 3′-hydroxyl termini in the double-strand DNA breaks generated during apoptosis.

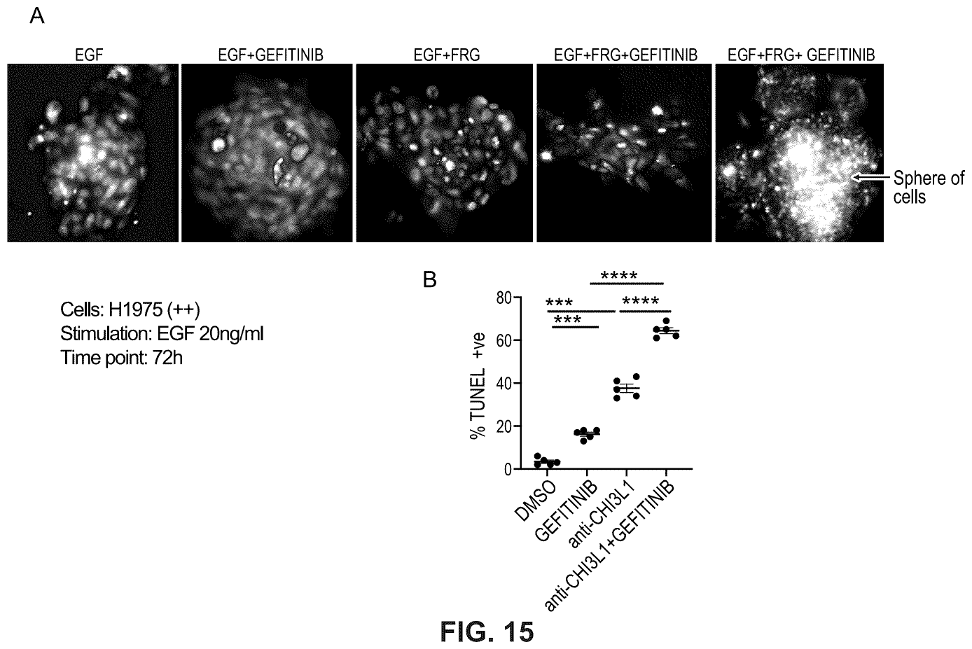

The results of the TUNEL staining can be seen histologically in FIG. 15A characterizing results in adherent cells and cells that formed spheroids. The TUNEL staining is quantitated in FIG. 15B. At the doses and time points noted, osimertinib and FRG individually were only modest stimulators of TUNEL staining. However, when used in combination significantly greater levels of TUNEL staining were noted.

These data demonstrate that gefitinib and FRG in combination interacted in a synergistic way to stimulate cell death and DNA injury and that these cell death effects were more potent than the effects induced by each moiety individually.

### Example 14 Effects of Anti-CHI3L1 (Frg) and Tki (Osimertinib), Alone and in Combination, on the Survival of H1975 Cells with “Common” EGFR Mutations

Studies were undertaken to define the effects of the TKI osimertinib and the anti-CHI3L1 FRG, alone and in combination, on DNA injury and cell death in EGF stimulated H1975 cells using TUNEL analysis.

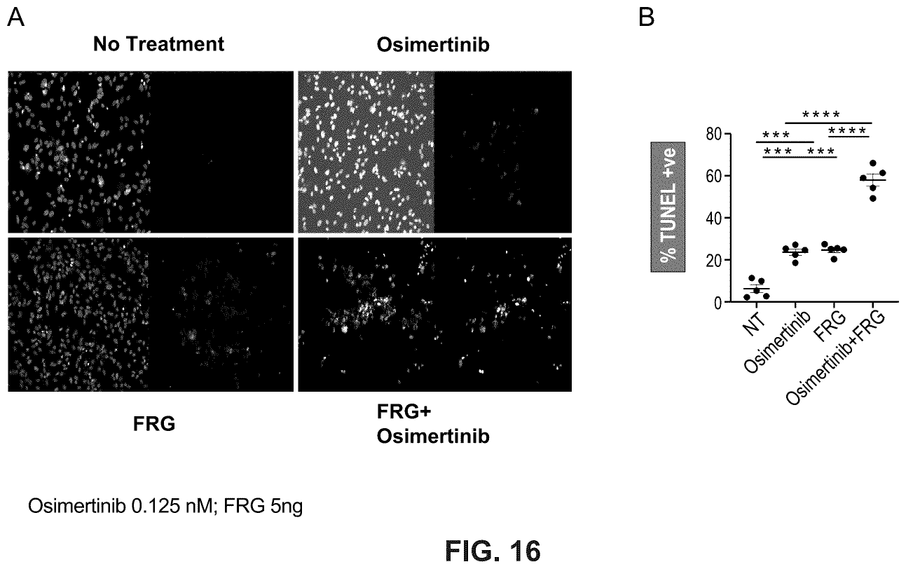

The results of the TUNEL staining can be seen in the cytochemistry panels characterizing results in adherent cells in FIG. 16A. The nuclei of these cells are highlighted by blue DAPI staining. The TUNEL staining is quantitated in FIG. 16B.

At the doses and time points noted, osimertinib and FRG individually were only modest stimulators of TUNEL staining. However, when used in combination significantly greater levels of TUNEL staining were noted. These data demonstrate that osimertinib and FRG in combination interacted in a synergistic manner to stimulate cell death and DNA injury and that these cell death effects were more potent than the effects induced by each moiety individually.

### Example 15 Effects of Anti-CHI3L1 (Frg) and Gefitinib, Alone and in Combination, on Pleural Colonies in B16-F10 Challenged Mice

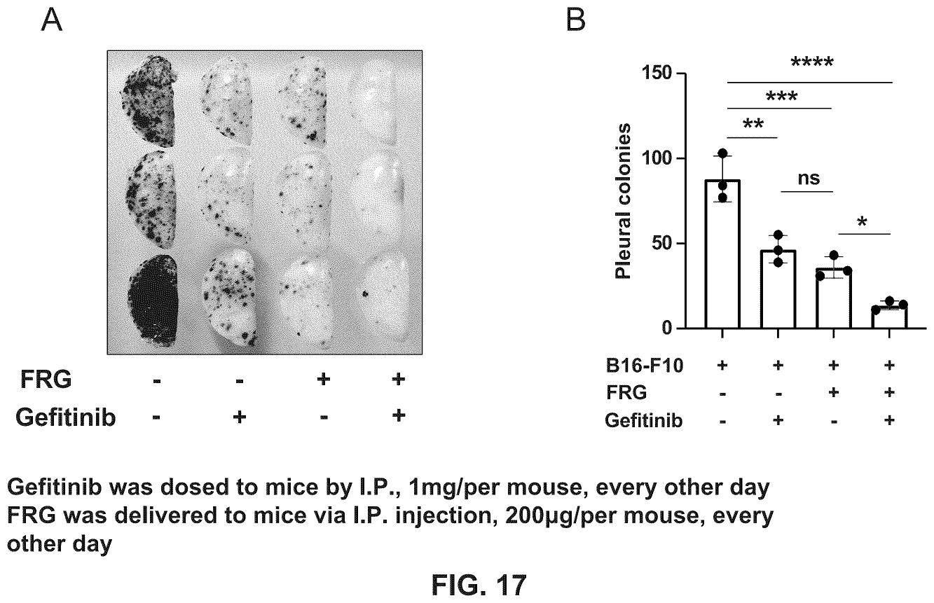

The B16F10 model was used in the present studies to define the effects of the TKI gefitinib and the anti-CHI3L1 FRG, alone and in combination, on the pulmonary metastasis of B16-F10 malignant melanoma cells. The B16-F10 melanoma cells were injected into tail veins of wild type C57BL/6 mice and their lungs were removed and evaluated visually (FIG. 17A) and quantitatively (FIG. 17B) 14 days later.

Gefitinib was a modest inhibitor and FRG was a strong inhibitor of melanoma metastasis. Importantly, these studies demonstrate that gefitinib and FRG in combination interact in a synergistic way to inhibit melanoma metastasis and that these inhibitory effects are more potent than the effects of each moiety individually.

### Example 16 CHI3L1 Stimulation of YAP1 and TAZ in Cells with WY EGFR Expression and “Common” EGFR Mutations

Yes-associated protein 1 (YAP1) and WW-domain-containing transcription regulator 1 (WWTR1; also known as and hereafter referred to as TAZ) are transcriptional coactivators with 46% amino acid identity, which are encoded by paralogous genes. Both proteins interact with the members of the transcriptional enhancer factor (TEA)-domain (TEAD) family of DNA-binding factors (TEAD1, TEAD2, TEAD3, TEAD4) to regulate transcription. YAP and TAZ can be regulated in response to multiple cellular and tissue-level signals through many mechanisms, including regulation of nuclear complexes, and shuttling between the nucleus and the cytoplasm, where they are inactive and/or degraded.⁴³

Studies were undertaken to define the effects of CHI3L1 on the expression of YAP1 and TAZ in epithelial cells with wild type (WT) EGFR (A549 cells, Calu3 cells) and cells with mutant EGFR (H1975 and HCC827). The cells were incubated with rCHI3L1 or its vehicle control (NT) for 72 hours and qRT-PCR was employed to assess YAP-1 and TAZ gene expression.

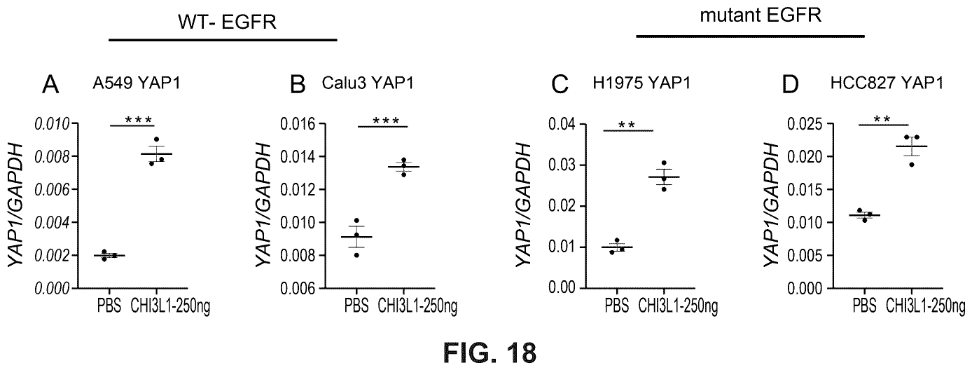

First, as shown in FIG. 18, rCHI3L1 (250 ng/mL) significantly stimulated YAP-1 in all cells tested: A549 cells (FIG. 18A, P<0.001), Calu3 cells (FIG. 18B, P<0.01), H1975 cells (FIG. 18C, P<0.01), and HCC827 cells (FIG. 18D, P<0.01).

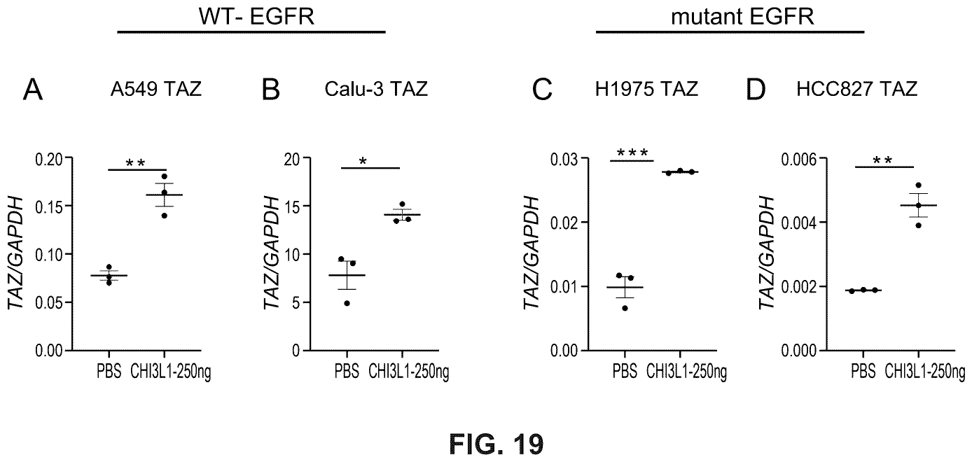

Second, as shown in FIG. 19, rCHI3L1 (250 ng/mL) also significantly stimulated TAZ in all cells tested: A549 cells (FIG. 19A, P<0.01), Calu3 cells (FIG. 19B, P<0.05), H1975 cells (FIG. 19C, P<0.001), and HCC827 cells (FIG. 19D, P<0.01).

### Example 17 CHI3L1 Stimulation of Tead1 in the Egfr Axis in Cells with WT EGFR Expression and “Common” Egfr Mutations

Studies were undertaken to define the effects of CHI3L1 on the expression of TEAD1 in epithelial cells with wild type (WT) EGFR (A549 cells, Calu3 cells) and cells with mutant EGFR (H1975 and HCC827). The cells were incubated with rCHI3L1 or its vehicle control (NT) for 72 hours and qRT-PCR was employed to assess TEAD1 gene expression.

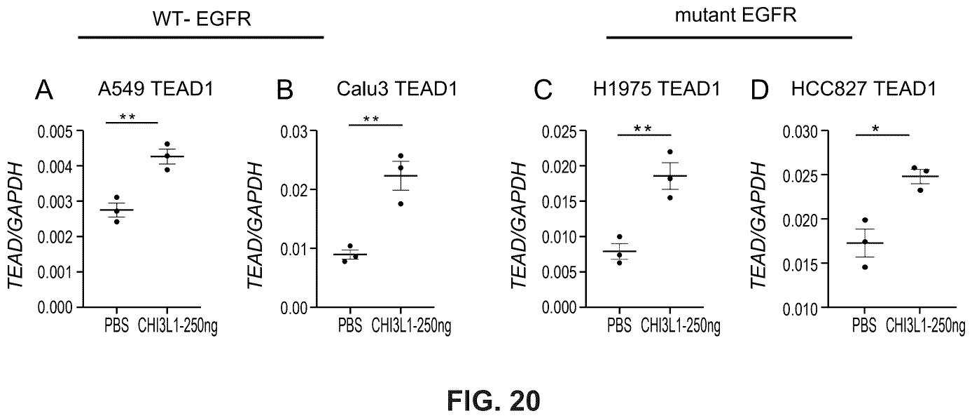

As shown in FIG. 20, rCHI3L1 (250 ng/mL) significantly stimulated TEAD1 in all cells tested: A549 cells (FIG. 20A, P<0.01), Calu3 cells (FIG. 20B, P<0.01), H1975 cells (FIG. 20C, P<0.01), and HCC827 cells (FIG. 20D, P<0.05).

### Example 18 Immunocytochemical (ICC) Demonstration of CHI3L1 Stimulation of Yap-1 in Nuclei from NSCLC Cells with Egfr “Common” Mutations

Double label immunocytochemistry (IHC) was used to define the accumulation and location of YAP1 in H1975 cells, which have mutant EGFR. These cells were treated for 72 hours with 250 ng/mL rCHI3L1 and 250 ng/mL FRG individually and in combination as noted. Nuclei were highlighted with DAPI staining (blue), cytoskeleton elements were highlighted in green with Phalloidin 488 (1:5000; Invitrogen #A12379) and YAP1 (1:500; R&D-NB110⁻⁵⁸³⁵⁸) was highlighted in red with Rabbit Alexafluor 596 (1:500).

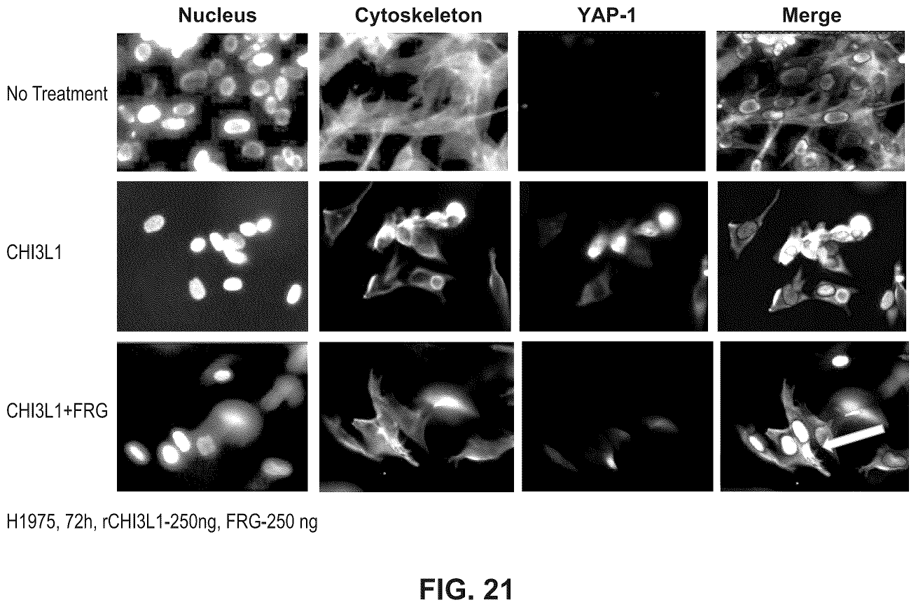

As shown in FIG. 21, YAP1 accumulation was not readily appreciated at baseline. When stimulated with rCHI3L1 (250 ng/mL), nuclear YAP-1 was significantly induced. Treatment with FRG diminished the accumulation of YAP1 in the nuclei and the YAP1 that was seen was largely in a cytoplasmic location (orange arrow).

### Example 19 Immunocytochemical (Icc) Demonstration of CHI3L1 Stimulation of Taz in Nuclei from NSCLC Cells with Egfr “Common” Mutations

Double label immunocytochemistry (ICC) was used to define the accumulation and location of TAZ in H1975 cells which have mutant EGFR. These cells were treated for 72 hours with 250 ng/mL rCHI3L1 and 250 ng/mL FRG individually and in combination as noted. Nuclei were highlighted with DAPI staining (blue), cytoskeleton elements were highlighted in green with Phalloidin 488 (1:5000; Invitrogen #A12379) and TAZ (1:500) (Invitrogen-703032) was highlighted in red with Rabbit Alexafluor 596(1:500).

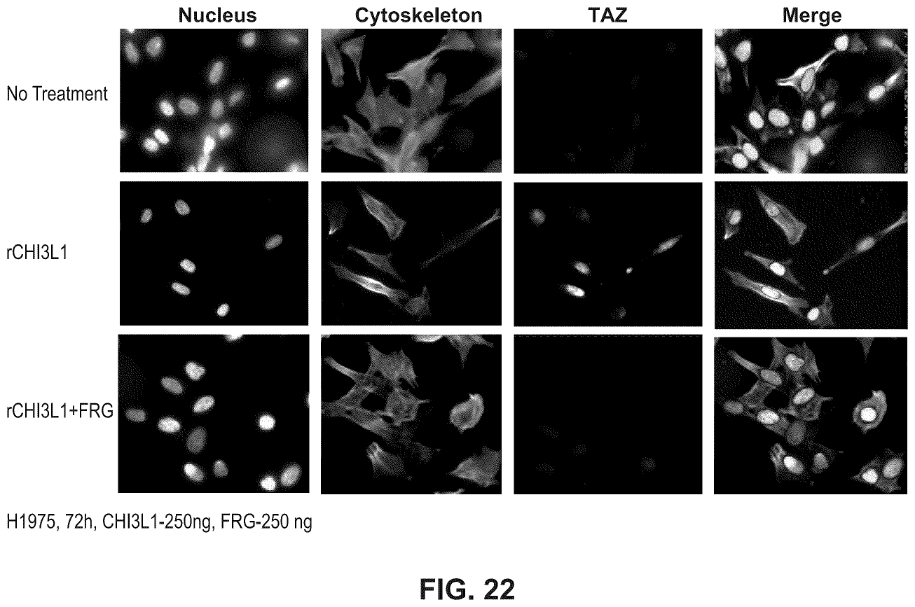

As shown in FIG. 22, TAZ accumulation was not readily appreciated at baseline. When stimulated with rCHI3L1, nuclear TAZ was significantly induced. Treatment with FRG diminished the accumulation of TAZ in the nuclei of these cells.

### Example 20 Immunocytochemical (ICC) Demonstration of CHI3L1 Stimulation of YAP-1 In Nuclei from Nsclc Cells with EGFR “Common” Mutations

Double label immunocytochemistry (IHC) was used to define the accumulation and location of YAP1 in HCC827 cells, which have mutant EGFR. These cells were treated for 72 hours with 250 ng/mL rCHI3L1 and 250 ng/mL FRG individually and in combination as noted. Nuclei were highlighted with DAPI staining (blue), cytoskeleton elements were highlighted in green with Phalloidin 488 (1:5000; Invitrogen #A12379) and YAP1 (1:500; R&D-NB110⁻⁵⁸³⁵⁸) was highlighted in red with Rabbit Alexafluor 596 (1:500).

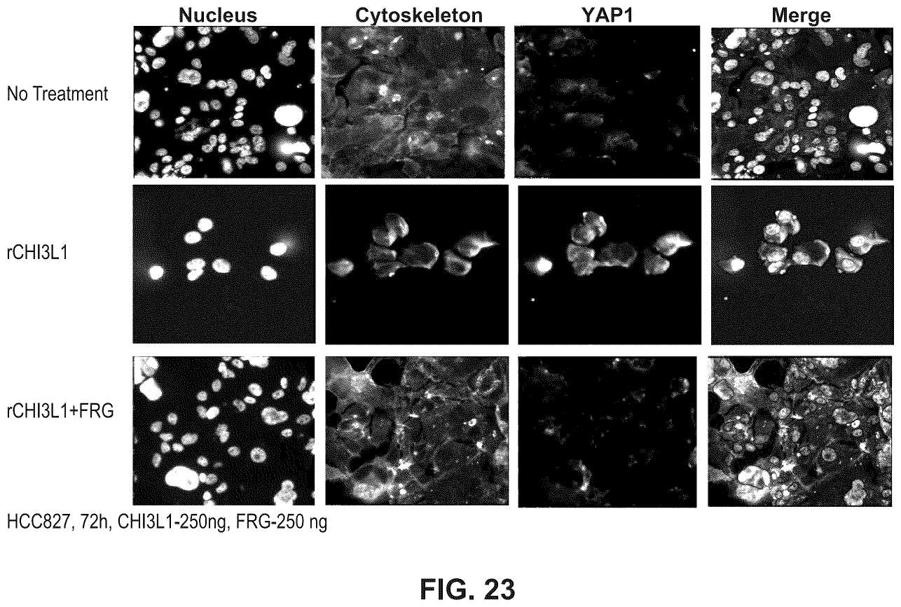

As shown in FIG. 23, YAP1 accumulation was not readily appreciated at baseline. When stimulated with rCHI3L1, nuclear YAP-1 was significantly induced. Treatment with FRG diminished this YAP-1 accumulation.

### Example 21 CHI3L1 Stimulation of Sox-Mediated Stemness in Cells with Wild Type and Mutant EGFR

Studies were undertaken to define the effects of and time course of rCHI3L1 on the expression of Sox-2 and Sox-9 in epithelial cells with wild type EGFR (Calu 3 cells) and cells with mutant EGFR (H1975). The cells were incubated with recombinant (r) CHI3L1 (250 ng/mL) or its vehicle control for up to 72 hours and qRT-PCR was employed to assess Sox-2 and Sox-9 gene expression.

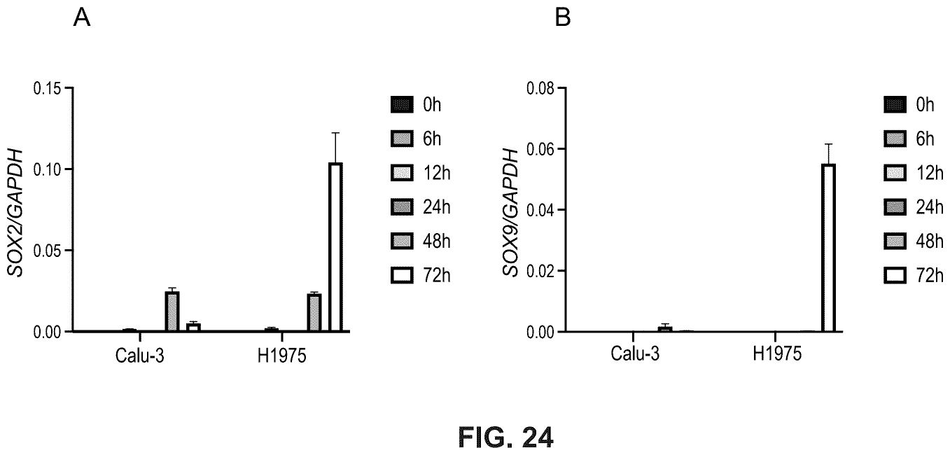

These data demonstrate that CHI3L1 stimulated Sox-2 (FIG. 24A) and Sox-9 (FIG. 24B) expression with the greatest stimulation occurring at 72 hours in cells with mutant EGFR.

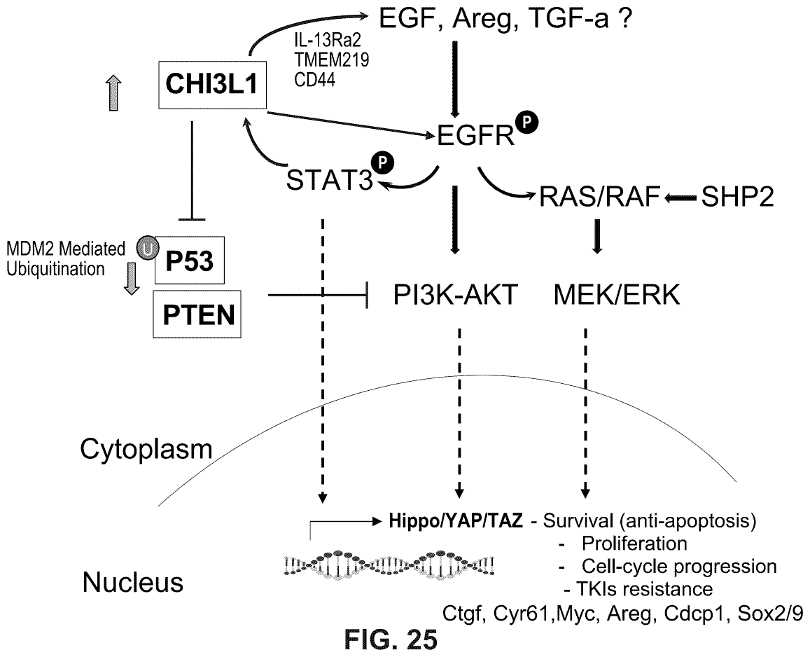

FIG. 25 schematically illustrates the contributions of CHI3L1 to EGFR signaling in tumor resistance and progression. EGFR activation stimulates CHI3L1 via Stat 3. CHI3L1 then feeds back to stimulate the expression and accumulation of EGFR and its ligands (EGF, AREG, TGF-α). CHI3L1 also feeds back to inhibit the tumor suppressors P53 and PTEN which in turn inhibit P13K and MAPK activation. This positive feedback loop also leads to activation of the Hippo/YAP-1/TAZ pathway, which contributes to cell survival, cell cycle progression and TKI resistance via down-stream genes such as Sox 2 and 9 which regulate stemness.

### Example 22 Kasugamycin Inhibits CHI3L1 Autoinduction and CHI3L1 Stimulation of Components of the EGFR Axis in EGRF Mutant Cells HCC827

Studies were undertaken to define the effects of the small molecule CHI3L1 inhibitor, kasugamycin (KSM), on CHI3L1 induction of CHI3L1 (CHI3L1 autoinduction) the CHI3L1 stimulation of the expression of EGFR and its physiologic ligand YAP-1. The studies compared the effects of recombinant (r) CHI3L1 (250 ng/mL) or its vehicle control on HCC827 cells (which have EGFR mutations) after 72 hours of treatment with and without KSM (500 ng/mL).

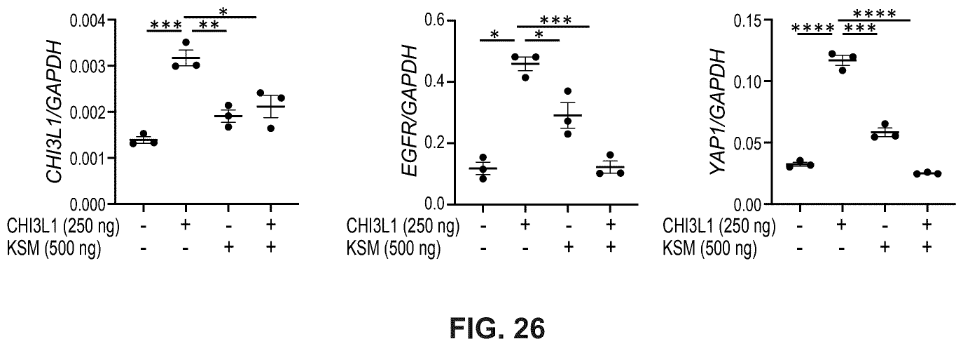

As shown in FIG. 26, rCHI3L1 (250 ng/mL) significantly stimulated CHI3L1 autoinduction (FIG. 26A; ***P<0.001) and the expression of EGFR (FIG. 26B; *P<0.05) and its downstream target YAP-1 (FIG. 26C; ****P<0.0001). KSM significantly inhibited the rCHI3L1-induced CHI3L1 autoinduction (FIG. 26A; *P<0.05), as well as the rCHI3L1-induced increased expression of EGFR (FIG. 26B; ***P<0.001) and its downstream target YAP-1 (FIG. 26C; ****P<0.0001).

### Example 23 Kasugamycin Inhibits CHI3L1 Autoinduction and CHI3L1 STIMULATION OF Components of the EGFR Axis in EGRF Mutant Cells H1975

Studies were undertaken to define the effects of the small molecule CHI3L1 inhibitor, kasugamycin (KSM), on CHI3L1 induction of CHI3L1 (CHI3L1 autoinduction) the CHI3L1 stimulation of the expression of EGFR and its physiologic ligands, TGF-α, and EGF, and the downstream target YAP-1. The studies compared the effects of recombinant (r) CHI3L1 (250 ng/mL) or its vehicle control on H1975 cells (which have EGFR mutations) after 72 hours of treatment with and without KSM (500 ng/mL).

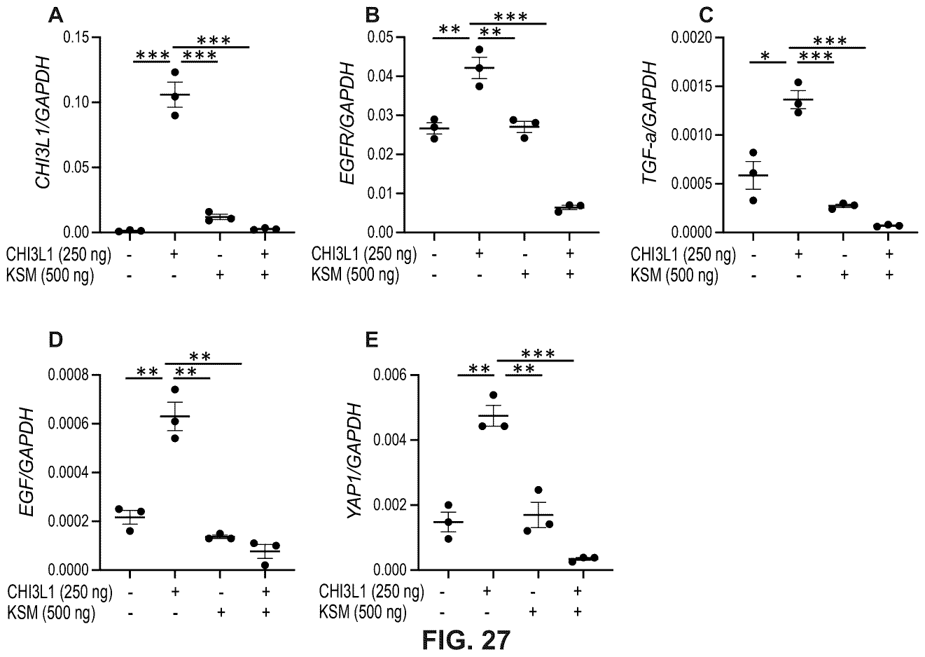

As shown in FIG. 27, rCHI3L1 significantly stimulated CHI3L1 autoinduction (FIG. 27A; ***P<0.001) and the expression of EGFR (FIG. 27B; **P<0.01) and its physiologic ligands, TGF-α (FIG. 27C; *P<0.05) and EGF (FIG. 27D; **P<0.01), and the downstream target YAP-1 (FIG. 26C; **P<0.01). Again, KSM significantly inhibited the rCHI3L1-induced CHI3L1 autoinduction (FIG. 27A; ***P<0.001), as well as the rCHI3L1-induced increased expression of EGFR (FIG. 27B; ***P<0.001) and its physiologic ligands, TGF-α (FIG. 27C; ***P<0.001) and EGF (FIG. 27D; **P<0.01), and the downstream target YAP-1 (FIG. 27E; ***P<0.001).

### Example 24 EGFR And CHI3L1 Axis in Lung Cancer

Studies were undertaken to further assess the essential role(s) played by CHI3L1 in the pathogenesis of the exaggerated activation of EGFR that is seen in EGFR-mutation driven NSCLC and establish that CHI3L1 is an important mediator in the development of therapeutic resistance in patients with NSCLC on TKI inhibitors such as gefitinib and osimertinib. This was accomplished by defining the effects of EGFR activation on CHI3L1 and the effects of CHI3L1 on EGFR and its physiologic and evaluating the effects of TKI inhibitors on the production of CHI3L1, the effects of anti-CHI3L1 and TKI, alone and in combination, on tumor progression and metastasis and the effects of anti-CHI3L1 on therapeutic resistance to osimertinib.

### Materials and Methods

### Cell Lines

Normal lung epithelial cells and lung cancer cell lines were purchased from the ATCC: NHBE (PCS-300-O10TM), A549 (CRM-CCL-185), H1975 (CRL-5908) and HCC827 (CRL-2868). Osimertinib-resistant HCC8270R2 cells were established according to the published protocol.⁴⁴ These osimertinib cells were maintained in a growth medium containing osimertinib (1 μM).

### Genetically Modified Mice

Lung-specific CHI3L1 overexpressing transgenic mice in which CHI3L1 was targeted to the lung with the CC10 promoter (CHI3L1 Tg) and CHI3L1 null mutant mice (Chil1^{-/-}) have been generated and characterized as previously described.^{45,46} These mice were between 6-12 weeks old when used in these studies. All animals were humanely anesthetized with Ketamine/Xylazine (100 mg/10 mg/kg/mouse) before any intervention.

### Western Blot Analysis

Protein lysates from cells were prepared with RIPA lysis buffer (ThermoFisher Scientific, Waltham, MA, USA) containing protease inhibitor cocktail (ThermoFisher Scientific) as per the manufacturer's instructions. 20 to 30 μg of lysate protein was subjected to SDS-PAGE electrophoresis on a 4-15% gradient mini-Protean TGX gel (Bio-rad, Hercules, CA, USA). It was then transferred to a membrane using a semidry method with a Trans-Blot Turbo Transfer System (Bio-Rad). Membranes were blocked with Tris-buffered saline with Tween20 (TBST) with 5% skimmed milk for 1 hour at room temperature. After blocking, the membranes were incubated with the primary antibodies overnight at 4 °C in TBST and 3% BSA. The primary antibodies used in this study are anti-(α) human CHI3L1 (R&D. AF2599-SP) and a-KEAP-1 (Santa Cruz Biotechnology. 8047), β-actin (Santa Cruz Biotechnology, Sc47778) antibodies. The membranes were then washed three times with TBST and incubated with secondary antibodies in TBST, 5% skimmed milk for 1 hour at room temperature. After three additional TBST washes, Supersignal West Femto Maximum Sensitivity Substrate Solution (Thermofisher Scientific) was added on to the membrane and immunoreactive bands were detected by using a ChemiDoc (Bio-Rad) imaging system.

RNA Extraction and Real-Time qPCR

Total cellular RNA was obtained using TRIzol reagent (ThermoFisher Scientific) followed by RNA extraction using RNeasy Mini Kit (Qiagen, Germantown, MD) according to the manufacturer's instructions. mRNA was measured and used for real time (RT)-PCR, as described previously.^{47,48} The primer sequences used in these studies are summarized in Table 5. Ct values of the test genes were normalized to internal housekeeping genes such as β-actin, GAPDH or RPL13a.

TABLE 5
Primer pairs used for qRT-PCR

| Human Gene | Forward primer | Ntd. | Reverse primer | Ntd. |
|---|---|---|---|---|
| GAPDH | GTCTCCTCTGACTTCAACAGCG | 22 | ACCACCCTGTTGCTGTAGCCAA | 22 |
| CHI3L1 | CCACAGTCCATAGAATCCTCGG | 22 | TGCCTGTCCTTCAGGTACTGCA | 22 |
| EGFR | AACACCCTGGTCTGGAAGTACG | 22 | TCGTTGGACAGCCTTCAAGACC | 22 |
| EGF | TTGGTGATGGGAGGATGACT | 20 | GGCCAGTGACTCAGCAGAAA | 20 |
| TGF-α | CTCAGACACATGCCTACACCT | 21 | GAACATCTCTGCTCGTCGCT | 20 |
| KEAP-1 | AGCGACGGTTCTACGTCCA | 19 | GCGACTGTCGGAAGTAGCC | 19 |
| P53 | CCTCAGCATCTTATCCGAGTGG | 22 | TGGATGGTGGTACAGTCAGAGC | 22 |
| Rb1 | AACATGCCCGACCCAATTACA | 21 | GTGTTTCTGCATACCTCATGG | 21 |
| PTEN | TGAGTTCCCTCAGCCGTTACCT | 22 | GAGGTTTCCTCTGGTCCTGGTA | 22 |
| Mouse Gene | Forward primer | Ntd. | Reverse primer | Ntd. |
| Rpl13a | AGGGGCAGGTTCTGGTATTG | 20 | TGTTGATGCCTTCACAGCGT | 20 |
| Chi3l1 | GCTTTGCCAACATCAGCAGCGA | 22 | AGGAGGGTCTTCAGGTTGGTGT | 22 |
| Egfr | GGACTGTGTCTCCTGCCAGAAT | 22 | GGCAGACATTCTGGATGGCACT | 22 |
| Egf | ACTGGTGTGACACCAAGAGGTC | 22 | CCACAGGTGATCCTCAAACACG | 22 |
| Tgf-α | TGATACGCCTGAGTGGCTGTCT | 22 | CACAAGAGCAGTGAGCGCTGAA | 22 |

### Measurement of Cellular Apoptosis and Cytotoxic Cell Death Responses

TUNEL staining using fluorescein-labelled dUTP was employed to assess DNA injury, apoptosis and cytotoxic cell death responses. After 72 hours incubation with test and control antibodies, cells were fixed in the 4% paraformaldehyde in PBS, permeabilized and blocked. In between each step, the cells were washed twice with PBS (1X). The cells were then stained with the in situ cell death detection kit, fluorescein (Roche, Mannheim, Germany) as per the manufacturer's instructions.

### Immunofluorescence Staining

To evaluate expression of CHI3L1, immunofluorescence (IF) staining was carried out using antibodies against CHI3L1 (FRG antibody). After 72 hours incubation period with test and control antibodies, cells were fixed in the 4% paraformaldehyde in PBS, permeabilized, and blocked. The cells were then incubated overnight at 40C with primary antibodies noted above, washed twice with PBS (1X) and incubated for 2 hours at 370C with secondary detection antibodies (1:500) and phalloidin (1: 3000) (Invitrogen, Waltham, MA) for cytoskeleton staining. The cells were then washed, mounted with VECTASHIELD® antifade mounting medium (Vector Laboratories Inc.) and evaluated at 20× via fluorescence microscopy.

### Melanoma Lung Metastasis and Antibody Treatment

B16-F10, a mouse melanoma cell line, was purchased from ATCC (Cat #: CRL-6475, Manassas, VA) and maintained in Dulbecco's Modified Eagles Medium (DMEM) supplemented with 10% Fetal Bovine Serum (FBS) and 1% penicillin and streptomycin. When the cells formed an 80% confluent monolayer, they were collected, adjusted to the concentration of 10⁶ cells/mL and injected into the mice via their lateral tail veins (2×10⁵ cells/mouse in 200 μL of DMEM). As previously described,⁴⁹ intraperitoneal injection of the noted doses of anti-CHI311 (FRG) antibody (50 and 200 μg/mouse) and different doses of gefitinib (1 mg/Kg) and Osimertinib (5 mg/Kg), alone and in combination and isotype control IgG (IgG2b) were started on the day of the B16 tumor cell challenge and continued every other day for 2 weeks. Metastasis was assessed and quantified by counting the melanoma colonies (black dots) on the pleural surface as previously described.^{50,51}

### Quantification and Statistical Analysis

Statistical evaluations were undertaken with GraphPad Prism software. As appropriate, groups were compared with 2-tailed Student's t test. Values are expressed as mean±SEM. One way ANOVA test were used for multiple group comparisons. Statistical significance was defined as a level of P<0.05.

### Results

### CHI3L1 Is Induced by Physiologic EGFR Activation

Studies were undertaken to define the relationship between CHI3L1 and the EGFR Axis. These investigations were initially undertaken to determine if EGFR activation altered the expression and production of CHI3L1.

In these experiments, we compared the levels of mRNA encoding CHI3L1 and the accumulation of CHI3L1 protein (YKL-40) in normal human bronchial epithelial cells (NHBE cells) incubated with medium alone or the physiologic ligands of EGFR.

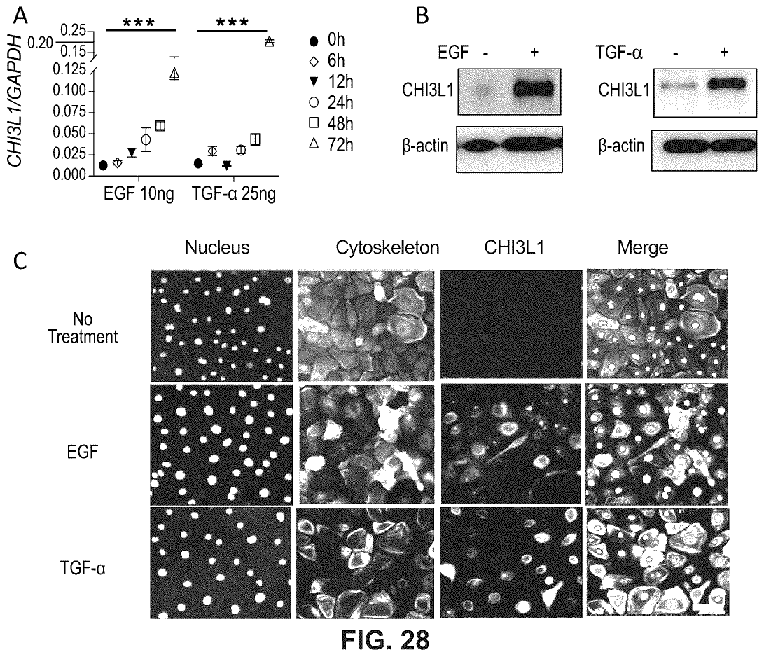

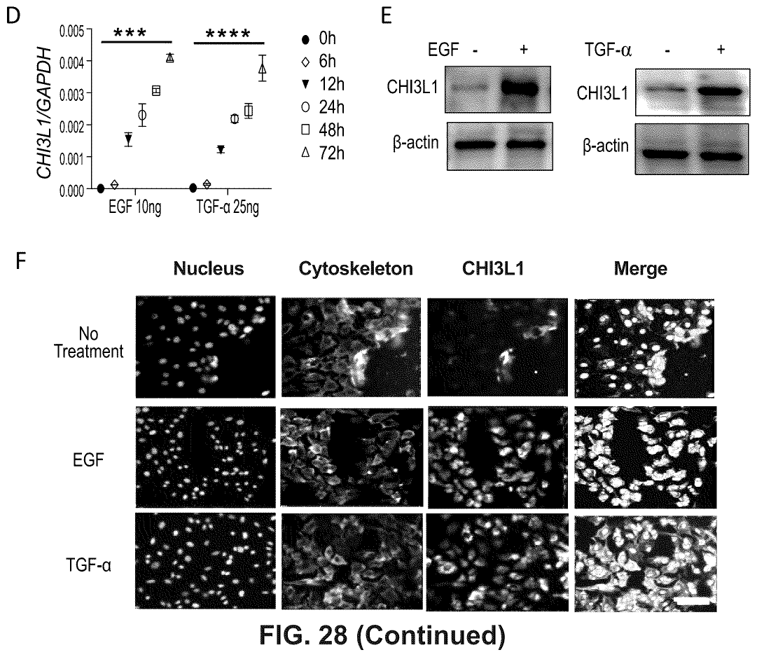

EGFR activation of normal epithelial cells by its physiologic ligands EGF and TGF-α stimulated the accumulation of CHI3L1 mRNA and protein (FIG. 28A-C and FIG. 29). The induction of CHI3L1 mRNA was appreciated after 24 hours and was more prominent after 48-72 hours of incubation (FIG. 28A). Similar responses were seen with transformed A549 epithelial cells which have wild-type EGFR. (FIG. 28D-F). These studies demonstrate that the activation of EGFR by its physiologic ligands stimulates CHI3L1 mRNA and protein accumulation.

CHI3L1 Is Produced by Cells with Mutant EGFR

Studies were next undertaken to define the effects of NSCLC-associated EGFR mutations on the production of CHI3L1.

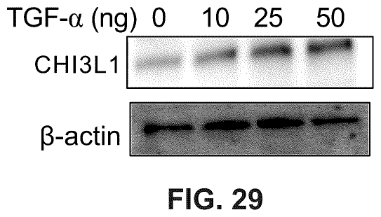

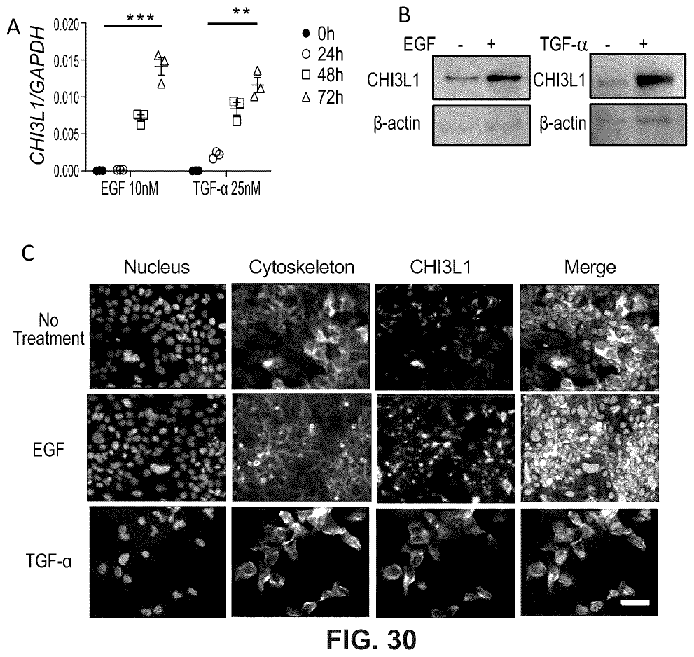

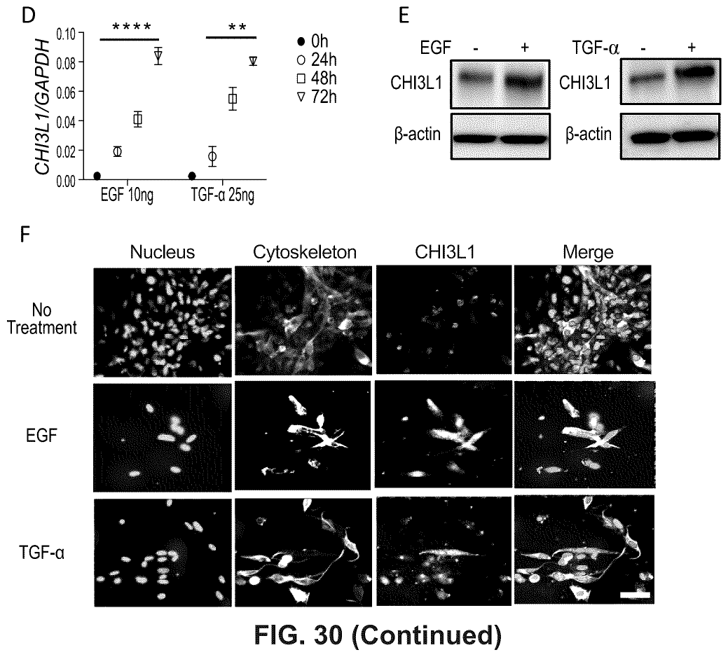

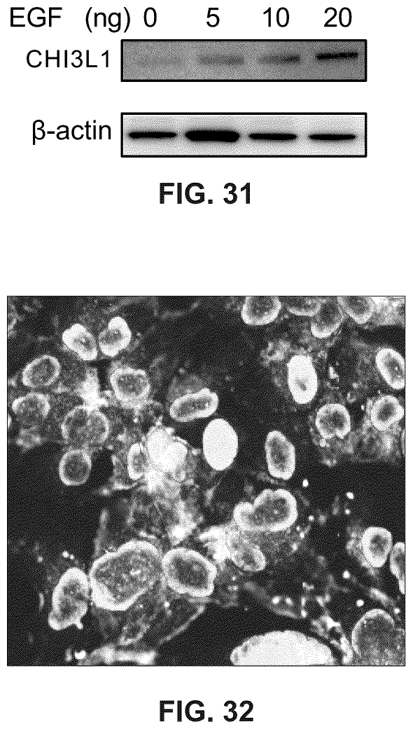

These experiments demonstrated that HCC827 cells, which have an exon 19 EGFR mutation,⁵² are potent producers of CHI3L1 mRNA and protein at baseline and that these events are exaggerated by EGF and or TGF-α (FIG. 30A-C). This stimulation was dose-dependent and most prominent after 48-72 hours of incubation with these physiologic ligands of EGFR (FIG. 30 and FIG. 31). Similar results were seen with immunohistochemistry, which highlighted the accumulation of nuclear and cytoplasmic CHI3L1 in HCC827 cells at baseline and the stimulatory effects of EGFR ligands on HCC827 cell CHI3L1 expression and production (FIG. 30C). H1975 cells, which have both the L858R and T790M mutations, had similar effects (FIG. 30D-F and FIG. 32). Importantly, cells with NSCLC-associated EGFR mutations produced more CHI3L1 than similarly stimulated NHBE cells or transformed cells with wild type EGFR like A549 cells (FIG. 33). Overall, these studies highlight significant accumulation and production of CHI3L1 in epithelial cells with mutant EGFR.

### CHI3L1 Forms a Positive Feedback Loop in the EGFR Axis

To further understand the relationships between CHI3L1 and EGFR, the effects of recombinant (r) CHI3L1 were next defined on components of the EGFR axis.

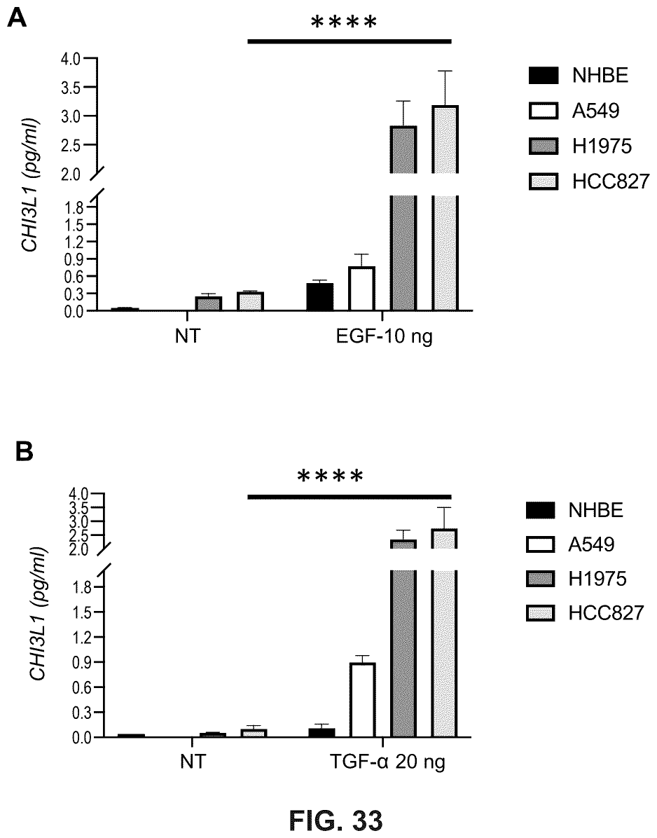

These in vitro studies demonstrated that rCHI3L1 is a potent, dose-dependent, stimulator of EGFR and its physiologic ligands EGF and TGF-α in cells with mutant EGFR (FIG. 34A-C). These stimulatory effects are CHI3L1-dependent because they were ameliorated by treatment with anti-CHI3L1 (FRG) antibodies (FIG. 34D-I). To determine if similar effects were seen in vivo, we compared the levels of mRNA encoding EGFR and its ligands in lungs from transgene negative and lung targeted CHI3L1 transgene positive mice. These studies demonstrated that CHI3L1 is a potent stimulator of EGFR and its ligands EGF and TGF-α in lungs from CHI3L1 transgenic mice (FIG. 34J-L).

Thus, these data suggest that rCHI3L1 and transgenic CHI3L1 are potent stimulators of EGFR and its ligands in cells with WT and mutant EGFR and lungs from transgenic mice, respectively. When viewed in combination, one can see how the ability of EGFR activation to stimulate the production of CHI3L1 and the ability of CHI3L1 to stimulate EGFR and its ligands sets up a CHI3L1-dependent, positive feedback loop in the EGFR axis.

CHI3L1 Is Auto Induced in Cells with WT and Mutant EGFR

Additional experiments were undertaken to compare the expression and accumulation of CHI3L1 in cells with WT or mutant EGFR that were stimulated with rCHI3L1.

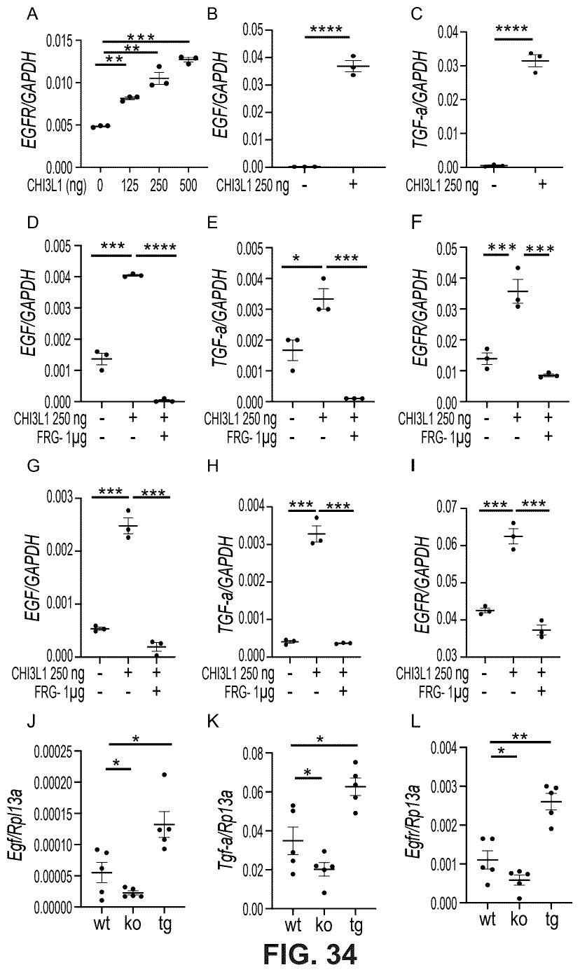

These studies demonstrated that CHI3L1 is auto induced by rCHI3L1 in NHBE cells, cells with WT EGFR (A549 cells) and cells with mutant EGFR. Similar results were seen in experiments with H1975 cells and HCC827 cells, which have differing NSCLC-associated EGFR mutations (FIG. 35A-C and FIG. 33).

The studies, as noted above, highlight a CHI3L1-driven positive feedback loop in the EGFR axis. These studies add to our understanding of this feedback loop by highlighting a CHI3L1 auto-induction mechanism that reinforces and feeds this feedback mechanism forward.

CHI3L1 Inhibitors and TKIs Interact in the Regulation of the EGFR Axis In vitro

TKI have become the first line of treatment for patients with EGFR mutant NSCLC. This appreciation started with first generation TKI, such as gefitinib or erlotinib.⁵³ Most recently, the FLAURA study demonstrated that osimertinib, a 3rd generation TKI, leads to better outcomes than earlier generation TKIs in the first-line therapy for advanced EGFR-mutant NSCLC with classical EGFR mutations.⁵⁴ To gain insight into these therapies, we characterized the effects of osimertinib on EGFR activation, CHI3L1 auto-induction, and the survival of H1975 and HCC827 cells, which have NSCLC-associated EGFR mutations.

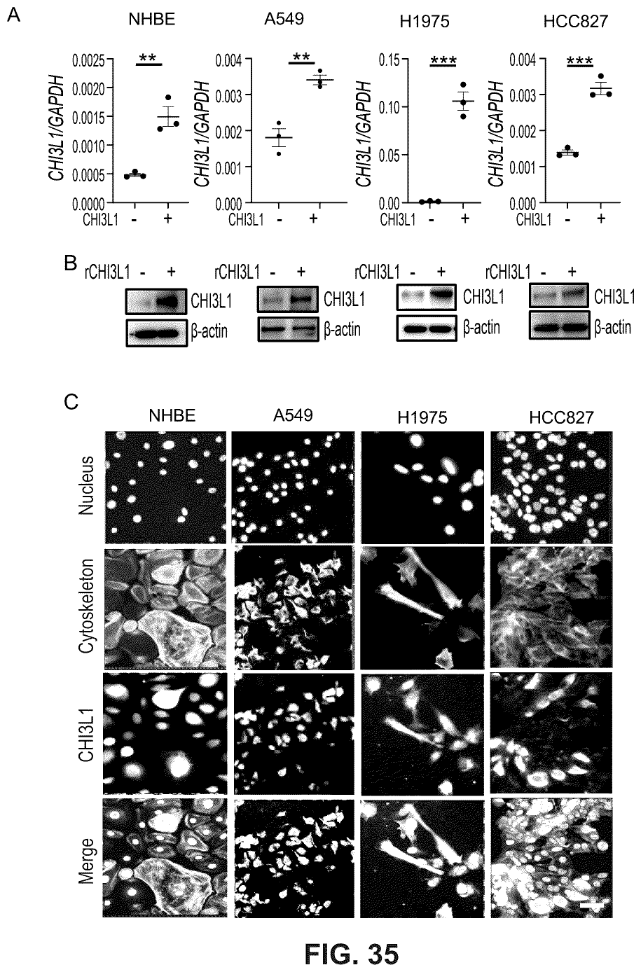

These studies demonstrated that, individually, osimertinib is a modest inhibitor and that anti-CHI3L1 (FRG) is a more potent inhibitor of CHI3L1 auto-induction and CHI3L1 stimulation of EGFR and EGF in these HC8227 cells (FIG. 36A-C). Interestingly, when osimertinib and FRG were used in combination, impressive interactions were appreciated, which were at least additive in nature (FIG. 36A-C). Impressive synergistic inhibition of CHI3L1 auto induction and EGF and EGFR expression were also seen when lower doses of osimertinib and FRG were employed FIG. 36D-E). In these experiments, lower doses of osimertinib (1 nM) and FRG (5 ng), that individually did not cause significant alterations in CHI3L1 autoinduction or EGF induction, were impressive inhibitors of CHI3L1 auto-induction and EGF when used in combination (FIG. 36D-E).

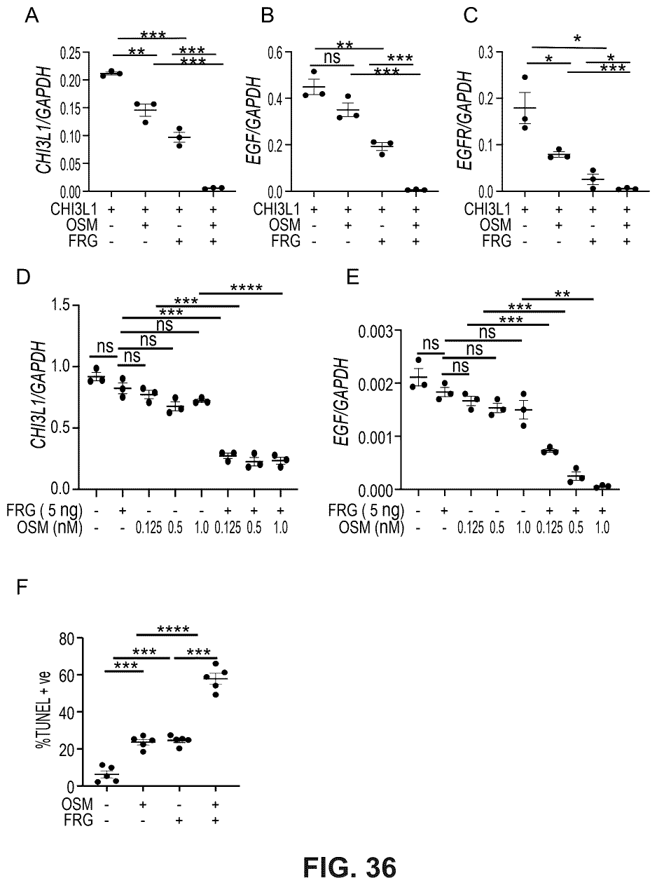

TUNEL evaluations of DNA cell injury were employed to if apoptotic cell death was involved in the combinatorial effects of gefitinib and FRG, alone or in combination. In these experiments, gefitinib and FRG individually caused cell apoptosis. Interestingly, when gefitinib and FRG were used in combination, impressive increases in cellular apoptosis were seen. (FIG. 37). Similar effects on apoptosis were seen with osimertinib and FRG, alone or in combination (FIG. 36F).

These studies demonstrate that TKI and anti-CHI3L1 (FRG) individually, and more potently in combination, inhibit CHI3L1 auto-induction and EGFR activation while inducing cellular apoptosis.

### Anti-CHI3L1 (FRG) Reverses Therapeutic Resistance to Osimertinib

A major rate limiting step in the treatment of EGFR mutant NSCLC is the development of resistance to the therapeutic agents. To gain insight into the relationship between this resistance and CHI3L1, we grew HCC827 cells in osimertinib until therapeutic resistance was acquired.⁵⁵ We then compared the gene expression and survival of these HCC8270R2 cells when incubated in the presence and absence of anti-CHI3L1 (FRG).

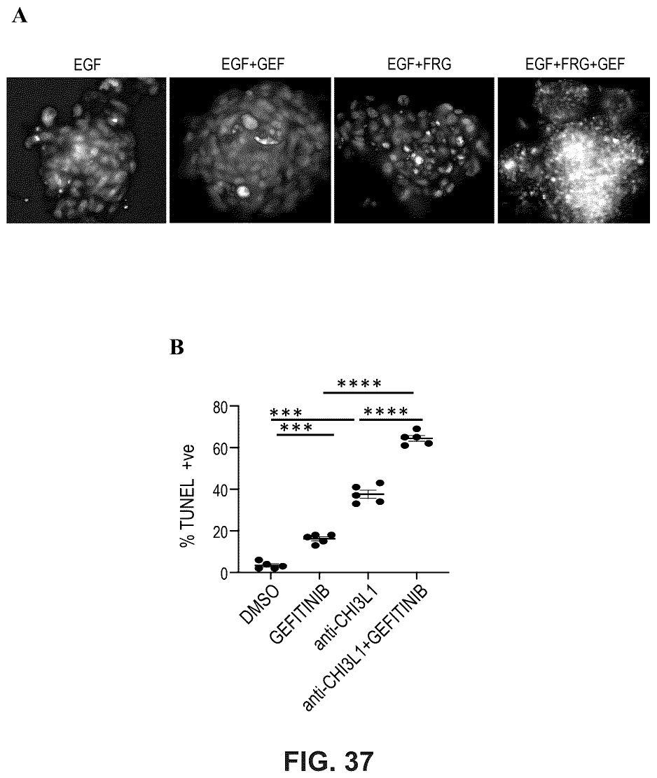

As can be seen in FIG. 38, osimertinib and FRG individually caused modest decreases in CHI3L1 auto-induction, and the expression of CHI3L1, EGF and EGFR (FIG. 38A). Importantly, the addition of FRG to cells in osimertinib elicited an impressive synergistic inhibition of the expression of CHI3L1, EGF, and EGFR (FIG. 38A). These effects were associated with similar changes in cell survival with osimertinib and FRG individually causing prudent increases in cellular apoptosis and the two therapeutic agents in combination causing an impressive synergistic increase in cell apoptosis (FIG. 38B). Kelch like ECH associated protein 1 (KEAP1) transcriptional activity has been associated with EGFR activation.⁵⁶ In accord with the ability of the tumor suppressor gene KEAP1 to modulate TKI sensitivity,⁵⁷ these experiments were associated with the opposite alterations in KEAP1 with osimertinib and FRG individually causing modest and osimertinib and FRG in combination causing increases in KEAP1 expression and protein accumulation (FIG. 38C and FIG. 38D). Similar results were seen with other tumor suppressor genes including retinoblastoma (RB1), P53 and PTEN (FIG. 38E).

These studies demonstrate that CHI3L1 inhibition reverses osimertinib resistance by inhibiting CHI3L1 auto-induction and inhibiting the expression of EGFR and its ligands while inducing apoptosis and stimulating production of KEAP1 and other tumor suppressors.

### CHI3L1 Inhibitors and TKI Inhibit Pulmonary Metastasis In Vivo

As noted above, malignancies with EGFR driver mutations frequently initially respond to and then fail to respond and develop metastasis as they develop TKI resistance. Thus, to further our understanding of TKI resistance and the relationship between FRG and TKI, we characterized the effects on pulmonary metastasis of TKI and FRG, alone and in combination in B16 cell challenged mice.

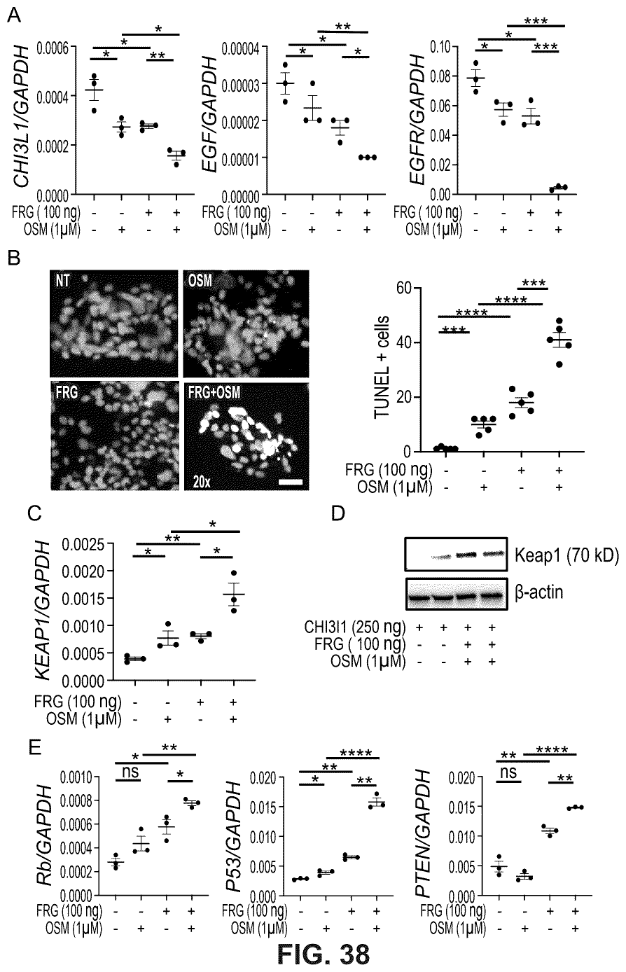

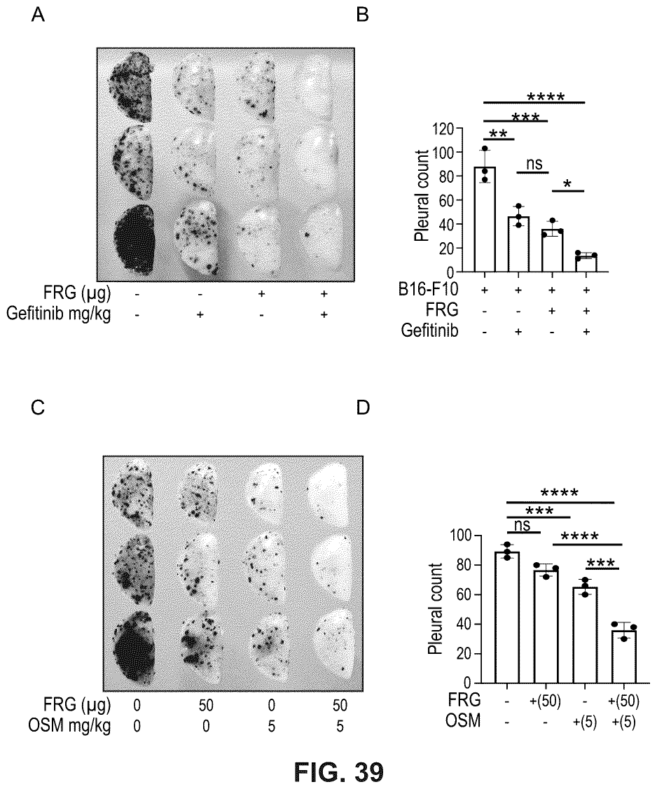

When used individually, FRG (intraperitoneally) and gefitinib or osimertinib (orally) inhibited B16-F10 pulmonary metastasis (FIG. 39A and FIG. 39C). When used in combination, at selected doses, impressive synergistic inhibition of tumor metastasis was seen (FIG. 39B and FIG. 39D). These studies demonstrate that anti-CHI3L1 and TKI, individually and more potently in combination, inhibit pulmonary metastasis.

### DISCUSSION

These studies demonstrate that EGFR activation stimulates the production of CHI3L1, that CHI3L1 feeds back to form a positive feedback loop by stimulating the production and expression of EGFR and its ligands and that this feedback is abrogated by TKI-based interventions. They also demonstrate that anti-CHI3L1 and TKIs inhibit EGFR activation and tumor metastasis, alone and more powerfully in combination. Lastly, they demonstrate that anti-CHI3L1 reverses therapeutic resistance that is seen during chronic osimertinib treatment via a mechanism that enhances the expression of KEAP1 and other tumor suppressors. These studies demonstrate that CHI3L1 is a critical therapeutic target, alone or in combination with TKIs, in the initial therapy and or salvage therapy of EGFR-mutant NSCLC.

### CONCLUSIONS

The above-described data in the present disclosure have:
1. Demonstrated that CHI3L1 interacts with and drives the expression and accumulation of EGFR and its activation in cells with wild type EGFR and EGFR mutations. This results in a positive feedback loop in which CHI3L1 drives EGFR activation and vice versa;
2. Identified a biomarker of EGFR mutant lung cancer progression;
3. Developed and characterized anti-CHI3L1 agents as a therapeutic in EGFR mutant NSCLC;
4. Demonstrated that anti-CHI3L1 agents, such as an anti-CHI3L1 antibody reagent or a small molecule CHI3L1 inhibitor, can interact in a synergistic manner with TKI in the treatment of EGFR mutant NSCLC;
5. Demonstrated that CHI3L1 is a powerful stimulator of the HIPPO/YAP/TAZ/TEAD pathway;
6. Demonstrated that the HIPPO/YAP/TAZ pathway contributes to the pathogenesis of TKI resistance in EGFR mutant NSCLC; and
7. Demonstrated that anti-CHI3L1inhibits the HIPPO/Yap/Taz pathway and can be used to treat TKI resistance in EGFR mutant NSCLC.
   The latter is very important because TKI resistance develops after approximately 8-12 months of TKI therapy and is the rate limiting step in the treatment of EGFR mutant NSCLC.

In summary, the present disclosure supports the use of anti-CHI3L1 (i.e., CHI3L1 inhibitors) for the treatment of EGFR mutant NSCLC alone or can be combined with, and work synergistically with, lower doses of TKI to treat EGFR mutant NSCLC and address the therapeutic resistance that develops in these patients when treated with TKIs alone.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following claims.

### REFERENCES

¹ Siegel, R. L., et al. (2021). “Cancer Statistics, 2021.” CA Cancer J. Clin. 71: 7-33.
² Hanna, N., et al. (2017). “Systemic Therapy for Stage IV Non-Small-Cell Lung Cancer: American Society of Clinical Oncology Clinical Practice Guideline Update.” J. Clin. Oncol. 35: 3484-3515.
³ Mansuet-Lupo, A., et al. (2016). “Intratumoral immune cell densities are associated with lung adenocarcinoma gene alterations.” Am. J. Respir. Crit. Care Med. 194: 1403-1412.
⁴ Lynch, T. J., et al. (2004). “Activating mutations in the epidermal growth factor receptor underlying responsiveness of non-small-cell lung cancer to gefitinib. N. Engl. J. Med. 350: 2129-2139.
⁵ Id.
⁶ Wang, D.-H., et al. (2017). “Progression of EGFR-Mutant Lung Adenocarcinoma is Driven by Alveolar Macrophages.” Clin. Cancer Res. 23: 778-788.
⁷ Ohashi, K., et al. (2013). “Epidermal Growth Factor Receptor Tyrosine Kinase Inhibitor-Resistant Disease.” J. Clin. Oncol. 31: 1070-1080.
⁸ Solassol, I., et al. (2019). “FDA-and EMA-Approved Tyrosine Kinase Inhibitors in Advanced EGFR-Mutated Non-Small Cell Lung Cancer: Safety, Tolerability, Plasma Concentration Monitoring, and Management.” Biomolecules 9(11) 668: 1-19.
⁹ Padlan, E. A., et al. (1995). “Identification of specificity-determining residues in antibodies.” FASEB J. 9(1): 133-139.
¹⁰ MacCallum, R. M., et al. (1996). “Antibody-antigen interactions: contact analysis and binding site topography.” J. Mol. Biol. 262(5): 732-745.
¹¹ Chothia, C. and Lesk, A. M. (1987). “Canonical structures for the hypervariable regions of immunoglobulins.” J. Mol. Biol. 196: 901-917.
¹² Chothia, C. (1989) “Conformations of immunoglobulin hypervariable regions.” Nature 342: 877-883.
¹³ Kabat et al. (1991) SEQUENCES OF PROTEINS OF IMMUNOLOGICAL INTEREST, Fifth Edition, U.S. Department of Health and Human Services, NIH Publication No. 91 3242.
¹⁴ Id.
¹⁵ See, e.g., U.S. Pat. Nos. 5,585,089; 6,835,823; and 6,824,989,
¹⁶ The Merck Manual of Diagnosis and Therapy, 19th Edition, published by Merck Sharp & Dohme Corp., 2011 (ISBN 978-O-911910⁻¹⁹-3).
¹⁷ The Encyclopedia of Molecular Cell Biology and Molecular Medicine, Robert S. Porter et al. (eds.), published by Blackwell Science Ltd., 1999-2012 (ISBN 9783527600908).
¹⁸ Molecular Biology and Biotechnology: A Comprehensive Desk Reference, Robert A. Meyers (ed.), published by VCH Publishers, Inc., 1995 (ISBN 1-56081-569-8).
¹⁹ Immunology by Werner Luttmann, published by Elsevier, 2006.
²⁰ Janeway's Immunobiology, Kenneth Murphy, Allan Mowat, Casey Weaver (eds.), Taylor & Francis Limited, 2014 (ISBN 0815345305, 9780815345305).
²¹ Lewin's Genes XI, published by Jones & Bartlett Publishers, 2014 (ISBN-1449659055).
²² Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414).
²³ Davis et al., Basic Methods in Molecular Biology, Elsevier Science Publishing, Inc., New York, USA (2012) (ISBN 044460149X).
²⁴ Laboratory Methods in Enzymology: DNA, Jon Lorsch (ed.) Elsevier, 2013 (ISBN 0124199542).
²⁵ Current Protocols in Molecular Biology (CPMB), Frederick M. Ausubel (ed.), John Wiley and Sons, 2014 (ISBN 047150338×, 9780471503385).
²⁶ Current Protocols in Protein Science (CPPS), John E. Coligan (ed.), John Wiley and Sons, Inc., 2005.
²⁷ Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737).
²⁸ See, e.g., PHYSICIANS' CANCER CHEMOTHERAPY DRUG MANUAL 2014, Edward Chu, Vincent T. DeVita Jr., Jones & Bartlett Learning; PRINCIPLES OF CANCER THERAPY, Chapter 85 in HARRISON'S PRINCIPLES OF INTERNAL MEDICINE, 18th edition; Therapeutic Targeting of Cancer Cells: Era of Molecularly Targeted Agents and Cancer Pharmacology, Chs. 28-29 in ABELOFFS CLINICAL ONCOLOGY, 2013 Elsevier; and Fischer D S (ed): THE CANCER CHEMOTHERAPY HANDBOOK, 4th ed. St. Louis, Mosby-Year Book, 2003).
²⁹ See, e.g., Maus et al. (2014). Blood, 123:2624-35; Reardon et al. (2014). Neuro-Oncology, 16:1441-1458; Hoyos et al. (2012). Haematologica, 97:1622; Byrd et al. (2014). Journal of Clinical Oncology, 32:3039-47; Maher et al. (2009). Cancer Research, 69: 4559-4562; and Tamada et al. (2012). Clinical Cancer Research, 18:6436-6445.
³⁰ See, e.g., Rockwood et al. (2011). Nature Protocols 6: 1612-1631; US Patent Publications 2011/0167602; 2011/0009960; 2012/0296352; and U.S. Pat. No. 8,172,901.
³¹ U.S. Pat. No. 5,317,095; Suzuki, Y. et al. “Alpha-D-glycosyl kasugamycin, its preparation, and antibacterial agent containing the same.” Issued: May 31, 1994. U.S. Pat. No. 4,554,269; Takaya, T. et al. “Kasugamycin derivatives, pharmaceutical compositions and method of use.” Issued: Nov. 19, 1985. U.S. Pat. No. 3,968,100; Umezawa, H. and Suhara, Y. “Guanidio derivatives of kasugamycin and their production.” Issued: Jul. 6, 1976.
³² REMINGTON'S PHARMACEUTICAL SCIENCES, 18^{th} edition, (1990). A. R. Gennaro (ed.), published by MackPubl., Easton, PA
³³ International Patent Publication WO 2019/036666; Elias, J. A. et al., “Methods and Compositions Relating to the Treatment of Fibrosis.” Published: Feb. 21, 2019.
³⁴ McNamara, J O., et al. (2006). Nat. Biotechnol. 24: 1005-1015.
³⁵ See, e.g., Kim SH., et al. (2008). Journal of Controlled Release 129(2): 10⁷-116.
³⁶ See, e.g., Ikeda and Taira (2006). Pharmaceutical Res 23:1631-1640; Soutschek et al. (2004). Nature 432:173-178 and Lorenze et al. (2004). Bioorg. Med. Chem. Lett. 14, 4975-4977.
³⁷ See, e.g., Maus et al. (2014). Blood 123: 2624-35; Reardon et al. (2014). Neuro-Oncology 16: 1441-1458; Hoyos et al. (2012). Haematologica 97: 1622; Byrd et al. (2014). J Clin Oncol 32: 3039-3047; Maher et al. (2009). Cancer Res 69:4559-4562; and Tamada et al. (2012). Clin Cancer Res 18:6436-6445.
³⁸ See, e.g., Slapak and Kufe, Principles of Cancer Therapy, Chapter 86 in HARRISON'S PRINCIPLES OF INTERNAL MEDICINE, 14th edition; Perry et al., Chemotherapy, Ch. 17 in ABELOFF, CLINICAL ONCOLOGY 2nd Edition, 2000 Churchill Livingstone, Inc; Baltzer L, Berkery R (eds))
³⁹ Lee, C. G., et al. (2009). “Role of breast regression protein 39 (BRP-39)/chitinase 3-like-1 in Th2 and IL-13-induced tissue responses and apoptosis.” J. Exp. Med. 206(5): 1149-1166.
⁴⁰ Sohn, M. H., et al. (2010). “The chitinase-like proteins breast regression protein-39 and YKL-40 regulate hyperoxia-induced acute lung injury.” American journal of respiratory and critical care medicine. 182(7): 918-928.
⁴¹ Ma, B., et al. (2015). “Role of chitinase 3-like-1 and semaphorin 7a in pulmonary melanoma metastasis.” Cancer Res. 75(3): 487-496.
⁴² Ma, B., et al. (2016). “RIG-like Helicase Regulation of Chitinase 3-like 1 Axis and Pulmonary Metastasis.” Sci. Rep. 6: 26299.
⁴³ Pocaterra, A., et al. (2020). “YAP/TAZ functions and their regulation at a glance.” Journal of Cell Science 133(2): 1-9.
⁴⁴ Tang, Z. H., et al. (2018). “Increased Expression of IRE1aAssociates with the Resistant Mechanism of Osimertinib (AZD9291)-resistant non-small Cell Lung Cancer HCC827/OSIR Cells.” Anticancer Agents Med. Chem. 18(4): 550-555.
⁴⁵ Lee, C. G., et al. (2009). “Role of breast regression protein 39 (BRP-39)/chitinase 3-like-1 in Th2 and IL-13-induced tissue responses and apoptosis.” J. Exp. Med. 206(5): 1149-1166.
⁴⁶ Lee, C. M., et al. (2016). “IL-13Ralpha2 uses TMEM219 in chitinase 3-like-1-induced signalling and effector responses.” Nat. Commun. 7: 12752.
⁴⁷ Lee, C. G., et al. (2009). “Role of breast regression protein 39 (BRP-39)/chitinase 3-like-1 in Th2 and IL-13-induced tissue responses and apoptosis.” J. Exp. Med. 206(5): 1149-1166.
⁴⁸ Sohn, M. H., et al. (2010). “The chitinase-like proteins breast regression protein-39 and YKL-40 regulate hyperoxia-induced acute lung injury.” American journal of respiratory and critical care medicine. 182(7): 918-928.
⁴⁹ Blakely, C. M., et al. (2017). “Evolution and clinical impact of co-occurring genetic alterations in advanced-stage EGFR-mutant lung cancers.” Nat. Genet. 49(12): 1693-1704.
⁵⁰ Ma, B., et al. (2015). “Role of chitinase 3-like-1 and semaphorin 7a in pulmonary melanoma metastasis.” Cancer Res. 75(3): 487-496.
⁵¹ Ma, B., et al. (2016). “RIG-like Helicase Regulation of Chitinase 3-like 1 Axis and Pulmonary Metastasis.” Sci. Rep. 6: 26299.
⁵² Mu, X., et al. (2013). “Ubiquitin ligase Cbl-b is involved in icotinib (BPI-2009H)-induced apoptosis and G1 phase arrest of EGFR mutation-positive non-small-cell lung cancer.” Biomed. Res. Int. 2013: 726375.
⁵³ Maemondo, M., et al. (2010). “Gefitinib or chemotherapy for non-small-cell lung cancer with mutated EGFR.” N. Engl. J. Med. 362(25): 2380-2388.
⁵⁴ Soria, J.-C., et al. (2017). “Osimertinib in Untreated EGFR-Mutated Advanced Non-Small-Cell Lung Cancer.” N. Engl. J. Med. 378(2): 113-125.
⁵⁵ Takahashi, A., et al. (2018). “Ankyrin Repeat Domain 1 Overexpression is Associated with Common Resistance to Afatinib and Osimertinib in EGFR-mutant Lung Cancer.” Scientific Reports 8(1): 14896.
⁵⁶ Huo, L., et al. (2014). “Activation of Keap1/Nrf2 signaling pathway by nuclear epidermal growth factor receptorin cancer cells.” Am. J. Transl. Res. 6(6): 649-663.
⁵⁷ Foggetti, G., et al. (2021). “Genetic Determinants of EGFR-Driven Lung Cancer Growth and Therapeutic Response In Vivo.” Cancer Discov. 11(7): 1736-1753.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating epidermal growth factor receptor (EGFR) mutant non-small cell lung cancer (NSCLC) in a subject in need thereof, the method comprising administering an inhibitor of Chitinase 3-Like 1 (CHI3L1).

2. The method of claim 1, wherein the inhibitor of CHI3L1 is an antibody, antibody reagent, antigen-binding fragment thereof, or chimeric antigen receptor (CAR) that specifically binds to the epitope of SEQ ID NO: 7.

3. The method of claim 2, wherein the inhibitor of CHI3L1 is an antibody comprising the complementarity determining regions (CDRs) of:
(a) a heavy chain CDR1 having the amino acid sequence of SEQ ID NO: 1;
(b) a heavy chain CDR2 having the amino acid sequence of SEQ ID NO: 2;
(c) a heavy chain CDR3 having the amino acid sequence of SEQ ID NO: 3;
(d) a light chain CDR1 having the amino acid sequence of SEQ ID NO: 4;
(e) a light chain CDR2 having the amino acid sequence of SEQ ID NO: 5; and
(f) a light chain CDR3 having the amino acid sequence of SEQ ID NO: 6, or conservative amino acid substituents thereof.

4. The method of claim 1, wherein the inhibitor of CHI3L1 is an antibody comprising a light heavy chain sequence of any one of SEQ ID NO: 8-20.

5. The method of claim 1, wherein the inhibitor of CHI3L1 is an antibody comprising a light chain sequence of any one of SEQ ID NO: 21-29.

6. The method of claim 1, wherein the inhibitor of CHI3L1 is an antibody comprising a heavy chain sequence of any one of SEQ ID NO: 8-20 and a light chain sequence of any one of SEQ ID NO: 21-29.

7. The method of claim 1, wherein the inhibitor of CHI3L1 is an inhibitor of CHI3L1 and chitinase 1.

8. The method of claim 7, wherein the inhibitor of CHI3L1 and chitinase 1 is kasugamycin (KSM) or a derivative, analog, or variant thereof.

9. The method of claim 7, wherein the inhibitor of CHI3L1 and chitinase 1 is KSM.

10. The method of claim 1, further comprising the administration of an EGFR-tyrosine kinase inhibitor (TKI).

11. The method of claim 10, wherein the antibody, antibody reagent, or antigen-binding portion thereof is conjugated to the EGFR-TKI.

12. The method of claim 10 or 11, wherein the EGFR-TKI is selected from the group consisting of: afatinib, erlotinib, gefitinib, olmutinib, osimertinib, rociletinib, cetuximab, panitumumab, dacomitinib, lapatinib, necitumumab, mobocertinib, and vandetanib.

13. The method of claim 10 or 11, wherein the EGFR-TKI is selected from the group consisting of: erlotinib, gefitinib, and osimertinib.

14.-25. (canceled)

26. The method of claim 1, wherein the subject is
i) afflicted with advanced EGFR mutant NSCLC and is no longer responding to TKI treatments,
ii) determined to have an elevated level of CHI3L1,
iii) determined to have a level of CHI3L1 that is increased compared to a prior assessment of the level in that subject, or
iv) determined to have a level of CHI3L1 that is increased compared to a reference level.

27.-29. (canceled)

30. The method of claim 1, wherein the CHI3L1 is circulating CHI3L1.

31. The method of claim 1, wherein the subject afflicted with metastatic EGFR mutant NSCLC is
i) determined to have a level of CHI3L1 that is increased compared to the subject's levels prior to metastasis or
ii) determined to have a level of CHI3L1 that is increased compared to the levels in patients with EGFR mutant lung cancer without metastasis.

32. (canceled)

33. A method of assessing the progression of EGFR mutant NSCLC using a biomarker in a subject afflicted with, or at risk of, EGFR mutant NSCLC using the subject's level of CHI3L1 as a biomarker, the method comprising assessing the level of CHI3L1 in the subject, wherein the level of CHI3L1 correlates directly with disease progression and inversely with disease-free interval and survival.

34. The method of claim 33, wherein the subject is
i) determined to have a level of CHI3L1 that is compared to a prior assessment of the level in that subject, or
ii) determined to have a level of CHI3L1 that is compared to a reference level.

35. (canceled)

36. The method of claim 33, wherein the CHI3L1 is circulating CHI3L1.

37. (canceled)
